(19) United States
(12) Patent Application Publication
(10) Pub. No.: US 2024/0189357 A1
(43) Pub. Date: Jun. 13, 2024

(54) CHIMERIC ANTIGEN RECEPTORS TO TARGET CD5-POSITIVE CANCERS

(71) Applicant: BOARD OF REGENTS, THE UNIVERSITY OF TEXAS SYSTEM, Austin, TX (US)

(72) Inventors: Katy REZVANI, Houston, TX (US); Elizabeth SHPALL, Houston, TX (US); Rafet BASAR, Houston, TX (US); May DAHER, Houston, TX (US); Sunil ACHARYA, Houston, TX (US); Nadima UPRETY, Houston, TX (US); Ana Karen NUNEZ CORTES, Houston, TX (US); Emily ENSLEY, Houston, TX (US); David MARIN COSTA, Houston, TX (US)

(21) Appl. No.: 18/552,727

(22) PCT Filed: Apr. 14, 2022
(86) PCT No.: PCT/US2022/024842

§ 371 Date: Sep. 27, 2023

Related U.S. Application Data
(60) Provisional application No. 63/174,990, filed on Apr. 14, 2021.

(51) Int. Cl. A61K 35/17; A61K 39/00; A61K 45/06; A61P 35/00; C07K 14/54; C07K 16/28; C12N 5/0783; C12N 9/22

(52) U.S. Cl. CPC A61K 35/17; A61K 39/4613; A61K 39/4631; A61K 39/464429; A61K 39/46444; A61K 45/06; A61P 35/00; C07K 14/5443; C07K 16/2896; C12N 5/0646; C12N 9/22; A61K 2239/13; A61K 2239/17; A61K 2239/21; A61K 2239/48; C12N 2310/20

(57) ABSTRACT

Embodiments of the disclosure include methods and compositions related to targeting of CD5-expressing cells with particular engineered receptors. In specific embodiments, NK cells are specifically engineered to bind the CD5 antigen using particular chimeric antigen receptor constructs. In certain embodiments, vectors that express the CD5-targeting CARs also express particular a suicide gene and/or one or more particular cytokines.

This application claims priority to U.S. Provisional Patent Application Ser. No. 63/174,990, filed Apr. 14, 2021, which is incorporated by reference herein in its entirety.

### TECHNICAL FIELD

Embodiments of the disclosure include at least the fields of cell biology, molecular biology, immunology, and medicine, including cancer medicine.

### BACKGROUND

T-cell malignancies in the relapsed refractory setting represent a group of hematologic malignancies with a particularly poor prognosis and novel therapies are direly needed. CD5 is a pan-T cell surface marker present in the majority of T-cell malignancies. It is also expressed by some B-cell malignancies such as mantle cell lymphoma (MCL) and chronic lymphocytic leukemia (CLL).^{1,2} On the other hand, CD5 expression by normal cells is restricted to thymocytes, peripheral T cells and a minor subpopulation of B-lymphocytes and it is not expressed by other hematopoietic cells. Therefore, CD5 is a great target antigen for adoptive CAR engineered cellular therapy. However, development of CAR-T cells targeting neoplasms of T-cell origin has been hindered by the shared expression of most targetable antigens on both malignant and normal T lymphocytes, which can promote CAR-T cell fratricide.³ Despite the issue with fratricide, CD5 CAR-T cells have been successfully developed and have shown preclinical efficacy⁴ and clinical safety and early signs of clinical efficacy in patients with T cell malignancies in a preliminary report.⁵ Nonetheless, using alternative immune effector cells that don't express CD5 on their surface would be an attractive approach since it will eliminate any concern of fratricide. NK cells are an attractive alternative as a platform for CAR engineering to target CD5 since they don't express this antigen on their surface. Another advantage of NK cells for CAR engineering is that, in contrast to T cells, they do not cause graft-versus-host disease (GVHD) in the allogeneic setting, opening the potential to produce a completely off-the-shelf cellular product to be used at point of care. A novel platform was developed to genetically modify CB-derived NK cells to express a CAR, ectopically produce IL-15 to support NK cell proliferation and persistence in vivo, and express a suicide gene, inducible caspase 9 (iC9), to address any potential safety concerns.⁶ These iC9/CAR19/IL15-NK cells demonstrated efficacy against B-cell malignancies in the pre-clinical and clinical settings.^{6,7} There has not been observed any cytokine release syndrome (CRS), immune effector cell-associated neurotoxicity syndrome (ICANS) or other toxicity associated with the CAR-NK cell product.⁷ The present disclosure concerns expansion of the CAR-NK cell platform to target other cancer antigens, including CD5, to broaden the clinical impact of this therapy.

Multiple studies have demonstrated that the choice of co-stimulatory domain in CAR-T cells influences their function, persistence and metabolic profile.⁸⁻¹⁰ In the field of CAR-NK cells, the first constructs tested in the clinic have incorporated CD28, a T cell specific co-stimulatory domain, borrowing from the design of CAR-T cells. There are some preclinical CAR-NK cell studies looking at the function of various NK specific co-stimulatory molecules, but there are no studies looking systematically at the impact of co-stimulatory domains on the proliferation, transcriptomic and proteomic profile, polyfunctionality, metabolism and fitness of the NK cells. In the present disclosure, it was aimed to do so, by comprehensively studying the impact of co-stimulatory domains including some that are more relevant for NK cell biology, namely DNAX-activating Protein 10 (DAP10), a major adaptor protein and the exclusive signaling intermediate of NKG2D in human NK cells, DNAX-activating Protein 12 (DAP12), an important adaptor molecule that associates with multiple activating receptors (e.g. NKG2C, NKp44, activating KIRs) and NKG2D, one of the most potent NK cytotoxicity receptors which is essential for anti-tumor immunity.¹¹ The results show that CD5 CAR-NK cells with DAP10 co-stimulatory molecule perform significantly better in vitro and in vivo and show enhanced polyfunctionality, metabolic fitness and memory like characteristics which augment their anti-tumor activity compared to CD5 CAR-NK cells containing other co-stimulatory molecules. Based on these preclinical data, a Phase I/II clinical trial evaluating the safety and efficacy of CD5-DAP10-CD3ζ CAR-NK cells is in preparation.

Thus, the present disclosure, in particular embodiments, concerns methods and compositions concerning genetical engineering of cells for adoptive cell therapy, including human NK cells, to target cancers including CD5-positive cancers.

### BRIEF SUMMARY

Embodiments of the disclosure encompass methods and compositions related to engineered cellular receptors that target CD5 (also known as cluster of differentiation 5 or LEU1 or T1, for example). In specific embodiments, the engineered receptors that target CD5 are in the form of polynucleotides, polypeptides, or are comprised on the surface of cells of any kind, including immune cells. In specific cases, the cells are immune cells, and in certain embodiments the immune cells are NK cells, NK T cells, invariant NKT cells, gamma delta T cells, regulatory T cells, B cells, macrophages, mesenchymal stromal cells (MSCs), dendritic cells, and so forth, from any source. In certain embodiments, reprogrammed NK cells from cord blood (CB-NK) are encompassed for targeting cancers expressing CD5 molecules.

CD5 is utilized as a target antigen for methods and compositions because it is expressed on multiple cancers, including chronic lymphoproliferative disorders/lymphomas, T-cell leukemia (T-ALL), T-cell lymphoma, and in some B cell malignancies such as chronic lymphocytic leukemia and mantle cell lymphoma. It is also expressed on thymic cancer, as well as thyroid, liver, colorectal and cervical cancer.

In particular embodiments, the adoptive cell therapy of the present disclosure, including the NK cell therapy, is particularly efficacious to allow eradication of cancer cells even upon re-exposure to cancer. Therefore, in some embodiments, an individual may be provided any cell therapy encompassed herein in the absence of detectable cancer in an individual. In specific cases, the individual may be at risk for cancer of any kind, such as at a higher risk than the general population. In some cases, the individual may have had one or more treatments for cancer and may or may not be considered cancer-free at the time of administration of the cell therapy of the present disclosure. Thus, in various embodiments, there is a method of preventing cancer or preventing metastasis of cancer in an individual, comprising the step of administering to the individual a therapeutically effective amount of any cells encompassed herein. The administering step may occur once or more than once. When occurring more than once, there may be any suitable duration of time between administrations, including 1-24 hours, 1-7 days, 1-4 weeks, 1-12 months, or 1, 2, 3, 4, 5, 6, 7, 8, 9, 10, or more years between administrations.

Some embodiments of CD5-targeting CAR T cells have been tested for the treatment of T-cell malignancies. However, CD5 is constitutively expressed by normal T cells as well as on T cell leukemia/lymphoma, and its use in CAR T cell therapy would require genetic deletion of the CD5 gene to prevent fratricide during CAR T manufacturing. In contrast, because normal NK cells do not express CD5, they will not be subject to fratricide. Thus, in particular embodiments of the disclosure, NK cells (including from cord blood (CB)) are reprogrammed NK cells to target cancers expressing CD5. The disclosure includes a number of novel CAR molecules including fusion of an scFv targeting human CD5 antigen with one or more activating signaling endodomains, including in some cases that incorporate either CD3ζ alone or in combination with costimulatory or adaptor signaling domains, such as NKG2D, 41BB, CD28, DAP10 and/or DAP12. In specific cases, allogeneic CB-NK cells are retrovirally transduced to express CD5 CAR.

In particular embodiments, immune cells of the disclosure encompassing CD5 CAR molecules also express one or more proteins that support their survival and proliferation. In specific cases, the immune cells are engineered to express one or more cytokines that facilitate the cells' expansion and persistence. In specific cases, the cytokine is interleukin 15 (IL-15), IL-2, IL-7, IL-12, IL-18 and/or IL-21 that aid in the survival and maintenance of the immune cells (e.g., NK cells). In certain aspects, a vector that encodes the CAR also encodes the cytokine, and each ultimately are produced as separate polypeptides.

Embodiments of the disclosure encompass a variety of novel, specific CAR constructs incorporating CD5 scFv heterologously fused to one or more signaling domains (including, for example, those comprising cytoplasmic portions of CD3ζ and one or more of CD28, DAP10, DAP12, and NKG2D). The scFv may comprise a fusion of the variable fragments derived from the heavy (V_{H}) and light (V_{L}) chains of a murine or human antibody with specificity for human CD5 antigen, in some cases. The vector also may comprise one or more cytokine genes, including the gene to produce human IL-1), IL-2, IL-21, IL-12, IL-7, IL-23, and/or IL-18 that aids in the survival and maintenance of the NK cells. As one example, CB-NK cells, thus modified, comprise a vector encoding CD5 scFv in a CAR that includes one or more costimulatory domains and CD3z in addition to IL15 that is produced as a separate molecule from the CAR.

Particular embodiments of the disclosure allow for the use of off-the-shelf immune cells, including at least NK cells, that are allogeneic with respect to a recipient individual, that target CD5-positive cells of any kind, and that also may or may not be transduced to express one or more cytokines, such as IL15, IL-2, IL-21, IL-12, IL-7, IL-23, and/or IL-18.

In specific embodiments of the disclosure, expression of one or more endogenous genes in the immune cell has been modified, for example the expression may be partially or fully reduced in expression. Although the modification may occur by any means, in specific embodiments expression of the one or more genes has been modified, such as by being reduced in expression levels, and this may occur by any suitable means including CRISPR or any other gene editing technology. Merely as examples, the endogenous gene may be selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, CD7, CTLA-4, TDAG8, CD38, CREM, and a combination thereof.

In one embodiment, there is an expression construct comprising sequence that encodes a CD5-specific engineered receptor and that optionally encodes one or both of the following: (a) a suicide gene; and (b) a cytokine. In specific cases, the CD5-specific engineered receptor is a chimeric antigen receptor (CAR) or a T cell receptor. The CD5-specific CAR may comprise a scFv having a heavy chain and a light chain, and wherein the heavy chain in the sequence that encodes the CAR is upstream of the light chain in a 5′ to 3′ direction. In other cases, the CD5-specific CAR comprises a scFv having a heavy chain and a light chain, and wherein the heavy chain in the sequence that encodes the CAR is downstream of the light chain in a 5′ to 3′ direction. In any case herein, the CD5-specific CAR does or does not comprise a codon-optimized scFv. In any case herein, the CD5-specific CAR does or does not comprise a humanized scFv. In any case herein, the CD5-specific CAR does or does not comprise a signaling peptide, such as one from CD8alpha, Ig heavy chain, or granulocyte-macrophage colony-stimulating factor receptor or a signal peptide derived from one or more other surface receptors. In particular embodiments, the CD5-specific CAR comprises one or more costimulatory domains, such as one or more costimulatory domains selected from the group consisting of CD28, CD27, OX-40 (CD134), DAP10, DAP12, 4-1BB (CD137), CD40L, 2B4, DNAM, CS1, CD48, NKG2D, NKp30, NKp44, NKp46, NKp80, ICOS, or a combination thereof.

Any CD5-specific CAR may or may not comprise CD3zeta and/or a hinge between the scFv and a transmembrane domain. In specific cases, the hinge is CD8-alpha hinge, the hinge comprises an artificial spacer comprised of Gly3, or the hinge comprises CH1, CH2, and/or CH3 domains of IgGs. In specific embodiments, the cytokine is IL-15, IL-12, IL-2, IL-18, IL-21, IL-7, or a combination thereof. In cases wherein a suicide gene is employed, the suicide gene may be a mutant TNF-alpha (such as an engineered nonsecretable mutant), inducible caspase 9, HSV-thymidine kinase, CD19, CD20, CD52, or EGFRv3.

Embodiments of the disclosure include immune cells of any kind comprising any expression encompassed herein. In specific embodiments, the immune cell is a NK cell, T cell, gamma delta T cell, invariant NKT (iNKT) cell, B cell, macrophage, MSC, dendritic cell, or mixture thereof. In cases wherein the immune cell is an NK cell, the NK cell may be derived from cord blood (including pooled cord blood units), peripheral blood, induced pluripotent stem cells, bone marrow, and/or from a cell line. In specific aspects, the NK cell line is NK-92 cell line or another NK cell line derived from a tumor or from a healthy NK cell or a progenitor cell.

In specific embodiments, the immune cell is an NK cell, such as one derived from cord blood, such as from a cord blood mononuclear cell. The NK cell may be a CD56+NK cell, in specific cases. The NK cells may express one or more exogenously provided cytokines, such as IL-15, IL-2, IL-12, IL-18, IL-21, IL-7, or a combination thereof. Particular embodiments include populations of immune cells of any kind of the disclosure, and the cells may be present in a suitable medium or a suitable carrier of any kind.

In one embodiment, there is a method of killing CD5-positive cells in an individual, comprising the step of administering to the individual an effective amount of cells harboring any expression construct encompassed by the disclosure. In specific embodiments, the cells are NK cells, T cells, gamma delta T cells, invariant NKT (iNKT) cells, B cells, macrophages, gamma delta T cells, or dendritic cells. NK cells may be derived from cord blood, peripheral blood, induced pluripotent stem cells, bone marrow, or from a cell line. NK cells may be derived from cord blood mononuclear cells. In some cases, the CD5-positive cells are cancer cells, including from hematopoietic cancers or solid tumors. The cells may be allogeneic or autologous with respect to the individual, who may or may not be a human. The cells may be administered to the individual by injection, intravenously, intraarterially, intraperitoneally, intratracheally, intrapleurally, intratumorally, intramuscularly, endoscopically, intralesionally, intracranially, percutaneously, subcutaneously, regionally, by perfusion, in a tumor microenvironment, or a combination thereof.

In particular embodiments of the methods, the cells may be administered to the individual once or more than once. The duration of time between administrations of the cells to the individual may be 1-24 hours, 1-7 days, 1-4 weeks, 1-12 months, or 1 or more years. The methods may further comprise the step of providing to the individual an effective amount of an additional therapy, such as surgery, radiation, gene therapy, immunotherapy, and/or hormone therapy. The additional therapy may comprise one or more antibodies or antibody-based agents, in some cases. In some aspects to the methods, they may further comprising the step of identifying CD5-positive cells in the individual.

Embodiments of the disclosure include expression constructs comprising sequence that encodes a CD5-specific engineered receptor, wherein said receptor comprises one of the following: (a) CD28 transmembrane domain (TM) and CD28 intracytoplasmic domain (ICD); (b) CD28 TM, CD28 ICD, and CD3zeta signaling domain; (c) CD28 TM and DAP12 ICD; (d) CD28 TM, DAP12 ICD, and CD3zeta signaling domain; (e) DAP12 TM and DAP12 ICD; (f) DAP12 TM, DAP12 ICD, and CD3zeta signaling domain; (g) CD28 TM and 4-1BB ICD; (h) CD28 TM, CD28 ICD, and 4-1BB ICD; (i) CD28 TM, CD28 ICD, 4-1BB ICD, and CD3zeta signaling domain; (j) CD28 TM and DAP10 ICD; (k) CD28TM, DAP10 ICD, and CD3zeta signaling domain; (l) DAP10 TM and DAP10 ICD; (m) DAP10 TM, DAP10 ICD, and CD3zeta signaling domain; (n) NKG2D ICD; (o) NKG2D TM; (p) NKG2D TM and NKG2D ICD; (q) NKG2D TM, NKG2D ICD, and CD3zeta signaling domain; (r) CD28 TM and NKG2D ICD; (s) NKG2D ICD, and CD3zeta signaling domain; (t) CD28 TM, NKG2D ICD, and CD3zeta signaling domain; (u) 4-1BB TM and 4-1BB ICD; (v) CD28TM and CD3zeta in the absence of an ICD that is a costimulatory domain; (w) CD8 TM and CD28 intracytoplasmic domain (ICD); (x) CD8 TM, CD28 ICD, and CD3zeta signaling domain; (y) CD8 TM and DAP12 ICD; (z) CD8 TM, DAP12 ICD, and CD3zeta signaling domain; (aa) CD8 TM and DAP12 ICD; (bb) CD8 TM, DAP12 ICD, and CD3zeta signaling domain; (cc) CD8 TM and 4-1BB ICD; (dd) CD8 TM, CD28 ICD, and 4-1BB ICD; (ee) CD8 TM, CD28 ICD, 4-1BB ICD, and CD3zeta signaling domain; (ff) CD8 TM and DAP10 ICD; (gg) CD8 TM, DAP10 ICD, and CD3zeta signaling domain; (hh) CD8 TM and DAP10 ICD; (ii) CD8 TM, DAP10 ICD, and CD3zeta signaling domain; (jj) CD8 ICD; (kk) CD8 TM; (ll) CD8 TM and NKG2D ICD; (mm) CD8 TM, NKG2D ICD, and CD3zeta signaling domain; (nn) CD8 TM and NKG2D ICD; (oo) CD8, NKG2D ICD, and CD3zeta signaling domain; (pp) CD8 TM, NKG2D ICD, and CD3zeta signaling domain; (qq) CD8 TM and 4-1BB ICD; or (rr) CD8TM and CD3zeta in the absence of an ICD that is a costimulatory domain. In some cases, the CD5-specific engineered receptor is a chimeric antigen receptor (CAR) or a T cell receptor. In cases wherein the CD5-specific engineered receptor is a CAR, the CAR may comprise a CD-targeting extracellular domain that is an scFv or a ligand for CD5. The CD5-specific CAR may comprise a scFv having a heavy chain and a light chain, and wherein the heavy chain in the sequence that encodes the CAR is upstream of the light chain in a 5′ to 3′ direction. The CD5-specific CAR may comprise a scFv having a heavy chain and a light chain, and wherein the heavy chain in the sequence that encodes the CAR is downstream of the light chain in a 5′ to 3′ direction. In specific cases, the CD5-specific CAR comprises a codon optimized scFv or comprises a humanized scFv. The scFv may be encoded by SEQ ID NO: 1 or SEQ ID NO:3. The scFv may comprise SEQ ID NO:2 or SEQ ID NO:4.

In particular embodiment, the CD5-specific CAR comprises a signaling peptide, such as one from CD8alpha, Ig heavy chain, granulocyte-macrophage colony-stimulating factor receptor, CD3 signaling peptide, CD4 signaling peptide, or a signal peptide derived from one or more other surface receptors.

In some embodiments of the CD5-specific CAR, the TM is encoded by SEQ ID NO:5, SEQ ID NO:7, SEQ ID NO:9, SEQ: 11 or SEQ ID NO: 13. The TM may comprise SEQ ID NO:6, SEQ ID NO:8, SEQ ID NO: 10, SEQ: 12 or SEQ ID NO:14. In cases wherein a specific ICD is utilized, it may be encoded by SEQ ID NO: 15, SEQ ID NO: 17, SEQ ID NO: 19, or SEQ ID NO:21. The ICD may comprise SEQ ID NO: 16, SEQ ID NO: 18, SEQ ID NO:20, or SEQ ID NO:22. In specific cases, the CD5-specific CAR comprises an ICD from CD27, OX-40 (CD134), CD40L, 2B4, DNAM, CS1, CD48, NKp30, NKp44, NKp46, NKp80, ICOS, or a combination thereof.

In specific embodiments, the CD5-specific engineered receptor is a CD5-specific CAR that comprises a hinge between a CD5-specific scFv and the transmembrane domain. The hinge may be an IgG hinge (such as IgG1 hinge, IgG2 hinge, IgG3 hinge, or IgG4 hinge, a CD28 hinge, a CD8-alpha hinge, the hinge comprises an artificial spacer comprised of Gly3, or the hinge comprises CH1, CH2, and/or CH3 domains of IgGs. The hinge may be encoded by SEQ ID NO:23 or 25 or may comprise SEQ ID NO:24 or SEQ ID NO:26.

The expression construct may encode a polypeptide of interest other than the CD5-specific engineered receptor and that may be separated on the construct by a 2A element (e.g., E2A, including one encoded by SEQ ID NO:31 or comprising SEQ ID NO:32) or an IRES element. The polypeptide of interest may be a therapeutic protein or a protein that enhances cell activity, expansion, and/or persistence. The polypeptide of interest may be a suicide gene (e.g., nonsecretable mutant TNF-alpha, inducible caspase 9, HSV-thymidine kinase, CD19, CD20, CD52, or EGFRv3), cytokine, human or viral protein that enhances proliferation, expansion and/or metabolic fitness, or a combination thereof. In certain embodiment, an expression construct encodes one or more cytokines in addition to the CD5-specific CAR, and the cytokine may be IL-15, IL-12, IL-2, IL-18, IL-21, IL-23, IL-7, or a combination thereof. When IL-15 is utilized, it may be encoded by SEQ ID NO:29 or comprise SEQ ID NO:30.

In specific cases, the expression construct encodes a CD5-specific CAR encoded by SEQ ID NO:33, SEQ ID NO:35, SEQ ID NO:37, SEQ ID NO:39, SEQ ID NO:41, SEQ ID NO:43, SEQ ID NO:45, or SEQ ID NO:47. The CD5-specific engineered receptor may be a CAR comprising SEQ ID NO:34, SEQ ID NO:36, SEQ ID NO:38, SEQ ID NO:40, SEQ ID NO:42, SEQ ID NO:44, SEQ ID NO:46, or SEQ ID NO:48.

Embodiments include any vectors that comprise any expression construct encompassed herein, including a viral vector (adenoviral vector, adeno-associated viral vector, lentiviral vector, or retroviral vector) or a non-viral vector (a plasmid, liposome, nanoparticle, lipid, carbohydrate, or combination thereof). Embodiments also include any immune cell that comprises an expression construct of the disclosure, and an immune cell may be natural killer (NK) cell, T cell, gamma delta T cells, invariant NKT (iNKT) cell, B cell, macrophage, MSCs, or dendritic cell. In cases wherein the immune cell is an NK cell, it may be derived from cord blood, peripheral blood, induced pluripotent stem cells, bone marrow, or from a cell line. The NK cell line may be NK-92 cell line or another NK cell line derived from a tumor or from a healthy NK cell or a progenitor cell. In specific cases, the NK cell is derived from a cord blood mononuclear cell. The NK cell may be a CD56+NK cell. The NK cell may express one or more exogenously provided cytokines, including IL-15, IL-2, IL-12, IL-18, IL-21, IL-23 IL-7, or a combination thereof. In the immune cells, one or more endogenous genes may be modified expression, such as partially or fully reduced in expression including using CRISPR. The endogenous gene may be selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, CD7, CTLA-4, TDAG8, CD38, CREM, and a combination thereof.

Embodiments of the disclosure include populations of immune cells described herein, said cells present in a suitable medium. The population may be housed in a storage facility, including in a frozen state, and the medium may comprise one or more cryoprotectants. In specific embodiments, the population of immune cells are NK cells that comprise an expression construct that encodes a CAR and IL-15. The CAR and IL-15 may be encoded by SEQ ID NO:33, SEQ ID NO:35, SEQ ID NO:37, SEQ ID NO:39, SEQ ID NO:41, SEQ ID NO:43, SEQ ID NO:45, or SEQ ID NO:47, and they may be comprised in SEQ ID NO:34, SEQ ID NO:36, SEQ ID NO:38, SEQ ID NO:40, SEQ ID NO:42, SEQ ID NO:44, SEQ ID NO:46, or SEQ ID NO:48.

Embodiments of the disclosure include methods of killing CD5-positive cells in an individual, such as one with CD5-positive cancer, comprising the step of administering to the individual an effective amount of cells harboring any expression construct encompassed herein. The cells may be immune cells such as NK cells, T cells, gamma delta T cells, invariant NKT (iNKT) cells, B cells, macrophages, gamma delta T cells, dendritic cells, or a mixture thereof. The NK cells may be derived from cord blood, cord blood mononuclear cells, peripheral blood, induced pluripotent stem cells, bone marrow, from a cell line, or a mixture thereof. The cells may be allogeneic or autologous with respect to the individual. The individual may be a mammal, such as a human. The cells may be administered to the individual once or more than once. The duration of time between administrations of the cells to the individual may be 1-24 hours, 1-7 days, 1-4 weeks, 1-12 months, or one or more years. Any treatment method may further comprise the step of providing to the individual an effective amount of an additional therapy, such as surgery, radiation, gene therapy, immunotherapy (including one or more antibodies), or hormone therapy. The cells may be administered to the individual by injection, intravenously, intraarterially, intraperitoneally, intratracheally, intratumorally, intramuscularly, endoscopically, intralesionally, intracranially, percutaneously, subcutaneously, regionally, systemically, by perfusion, in a tumor microenvironment, or a combination thereof. The method may further comprise the step of identifying CD5-positive cancer in the individual and/or producing the cells harboring the expression construct.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1H. Cord blood NK cells can be successfully transduced with CD5-CD28-CD3ζ and CD5-41BB-CD3ζ CAR constructs and can target CD5+ tumor cells but do not lead to long-term cures in vivo. 1A. maps of CD5-CD28-CD3ζ and CD5-41BB-CD3ζ CAR constructs. 1B. Bar graph showing the transduction efficiency of NK cells transduced with CD5-CD28-CD3ζ and CD5-41BB-CD3ζ CAR constructs compared to non transduced (NT) NK cells (from left to right, the bars represent non-transduced cells, cells with CD5CAR 41BB-CD3z, and cells with CD5CAR CD28-CD3z). 1C. Graph showing the fold change expansion of CD5-CD28-CD3ζ and CD5-41BB-CD3ζ CAR-NK cells compared to NT NK cells, here day 0 indicates the day of transduction. 1D. Bar graph showing percentage of cytokine production (IFNg, TNFa) and degranulation (CD107a) of CD5-CD28-CD3ζ and CD5-41BB-CD3ζ CAR-NK cells compared to NT NK cells after co-culture with CD5+CCRF-CEM tumor for 6 hours (from left to right, the bars represent non-transduced cells, cells with CD5CAR 41BB-CD3z, and cells with CD5CAR CD28-CD3z). 1E. Graph showing percent lysis of CD5+CCRF-CEM after co-culture with CD5-CD28-CD3ζ or CD5-41BB-CD3ζ CAR-NK cells or NT NK cells for 4 hours (NT is on the bottom, and CD5CAR CD28-CD3z is on the top). 1F. Bioluminescent imaging (BLI) showing tumor growth over time in mice engrafted with CD5+CCRF-CEM tumor transduced with fireflyluciferase (Ffluc) and either left untreated or treated with NT NK cells or with CD5-CD28-CD3ζ or CD5-41BB-CD3ζ CAR-NK cells. 1G. Graph plotting average radiance of BLI data comparing the different groups of mice shown in the IF panel (CCRF-CEM alone is to the left, and CCRF-CEM+CD5CAR CD28-CD3z is to the right). 1H. Graph showing survival curves of the different groups of mice shown in the 1F panel (CCRF-CEM alone is to the left, and CCRF-CEM+CD5CAR CD28-CD3z is to the right).

FIGS. 2A-2E. Optimizing CD5 CAR construct design by incorporating co-stimulatory molecules relevant for NK cell biology. 2A. Maps of the various CD5 CAR constructs with different transmembrane domains and co-stimulatory domains. 2B. Bar graph showing the transduction efficiency of NK cells transduced with the various CD5 CAR constructs compared to non transduced (NT) NK cells. 2C. Graph showing the expansion (cell counts in millions over time) of the various CD5 CAR-NK cell products compared to NT NK cells, here day 0 indicates the day of transduction. 2D. Bar graph showing percentage of cytokine production (IFNg, TNFa) and degranulation (CD107a) of the various CD5 CAR-NK cells compared to NT NK cells after co-culture with CD5+CCRF-CEM tumor for 6 hours. 2E. Graph showing percent lysis of CD5+CCRF-CEM after co-culture with the various CD5 CAR-NK cells or NT NK cells for 4 hours.

FIGS. 3A-3E. CD5 CAR-NK cells with DAP10 costimulatory domain improve tumor control significantly compared to CD5 CAR-NK cells with CD28 or DAP12 costimulatory domains. 3A. Bioluminescent imaging (BLI) showing tumor growth over time in mice engrafted with CD5+CCRF-CEM tumor transduced with fireflyluciferase (Ffluc) and either left untreated or treated with NT NK cells or with CD5-CD28-CD3ζ or CD5-DAP12-CD3ζ or CD5-DAP10-CD3ζ CAR-NK cells. 3B. Graph plotting average radiance of BLI data comparing the different groups of mice shown in the 3A panel, data from each mouse is plotted separately with mice belonging to the same group labeled in different colors (High CD5 CCRF-CEM alone are the lines to the left; CCRF-CEM+CD5 CAR CD28CD3z are the lines to the right). 3C. Graph showing survival curves of the different groups of mice shown in the 3A panel. 3D. Graph showing the body weights of mice in the various groups described in panel 3A, data from each mouse is plotted separately with mice belonging to the same group labeled in different colors (NT is the vertical line to the left; CCRF-CEM+CD5 CAR CD28CD3z is the vertical line to the right; CCRF-CEM+CD5 CAR DAP10CD3z is the nhorizontal line at top by itself after about 40 days). 3E. CD5 CAR-NK cells with DAP10-CD3z signaling show superior activity against T-ALL cell line even with low CD5 expression. Graph showing survival curves of the different groups of mice engrafted with CCRF-CEM tumor with low CD5 expression and then either left untreated or treated with non transduced (NT) NK cells or CD5 CAR-NK cells with various designs (CD5-41BB-CD3ζ, CD5-DAP12-CD3ζ, CD5-CD28-CD3ζ, CD5-DAP10-CD3ζ) (Low CD5 CRF alone is the vertical line furthest left; Low CD5 CCRF-CEM+CD5CAR DAP10-Cd3z is the horizontal line at top; Low CD5 CCRF-CEM_CD5CAR CD28-Cd3z is the short vertical line furthest right ending at about 60% survival).

FIGS. 4A-4B. CD5 CAR-NK cells with DAP10 signaling improve outcomes in a PDX mouse model of CD5+ mantle cell lymphoma. 4A. Schematic diagram of the mouse experimental plan showing that on day 0 mice are injected subcutaneously with the PDX cell line, on day 21 mice are injected via tail vein with the CD5-DAP10-CD3z CD5 CAR-NK cells with DAP10 costimulation and on day 60 they were sacrificed and tissues were analyzed by flow cytometry. 4B. Bar graphs showing the absolute number of CD45+CD5+ tumor cells in the subcutaneous tumor, spleen and bone marrow in mice that received no treatment or treated with CD5 CAR with DAP10 costimulation (Tumor alone is the bar to the left).

FIGS. 5A-5C. CD5 CAR-NK cells with DAP10-CD3z signaling show enhanced activation profile on the proteomic level. 5A. TSNE plot showing the various clusters from NT and CD5 CAR-NK cells combined together. 5B. TSNE plots showing clusters in the non-transduced (NT) NK cells and CD5 CAR-NK cells with DAP10 costimulation. Clusers 8 and 11 seem to be specific to CD5 CAR-NK cells with DAP10 costim. 5C. Heatmap showing the various makers of NK cells on the X axis and the various clusters on the Y axis. This shows that clusters 8 and 11 which are specific to CD5 CAR-NK cells with DAP10 costim are enriched in NK cell activation markers (DNAM, NKG2D, CD69, maturation markers (EOMES and T-BET) and cytotoxicity markers (Perforin, Granzyme B and TRAIL).

FIGS. 6A-6D. CD5 CAR-NK cells with DAP10 co-stimulatory domain demonstrate enhanced polyfunctionality and metabolic fitness. 6A. Bar graph showing polyfunctionality of NK cells that are either non transduced (NT) or transduced with various CD5 CAR-NK cells (with no costim (CD3z alone) or the various costimulatory domains after stimulation with CD5+ antigen, this shows the percentage of NK cells that are producing either 2, 3, 4 or 5+ proteins at the single cell level in response to antigen stimulation. 6B. Polyfunctionality heatmap showing the production of cytokines at the single cell level. Each row represents an NK cell condition, each column represents a cluster of NK cells that produces a specific set of cytokines, The more clusters there are the more polyfunctional the NK cells are. 6C. Bar graph showing the polyfunctionality strength index, showing what proportion of the NK cells (NT or the various CD5 CAR-NK cell designs) produce effector, stimulatory or chemoattractive cytokines at the single cell level (chemoattrative scoring is at the top of the bars and effector scoring is at the bottom of the bars). 6D. Graph showing oxygen consumption rate (OCR) of NT NK cells or NK cells transduced with the various CD5 CAR-NK cell constructs. This shows that CD5 CAR-NK cells with DAP10 costimulatory domain have the highest OCR which is a measure of mitochondrial activity and metabolic fitness.

FIGS. 7A-7C. CD5 CAR-NK cells with DAP10-CD3z signaling domain display memory-like characteristics and continue to kill CD5+CCRF-CEM after multiple tumor rechallenges. 7A. Schematic diagram detailing the experimental plan for the Incucyte real time cytotoxicity assay with multiple CD5+CCRF-CEM tumor rechallenges. Tumor cells are labeled with a red dye to be followed with real time imaging. 7B. Graph showing the red count over time which correlates with burden of live tumor cells. This shows that at the beginning all NK cells even non transduced (NT) NK cells are able to kill the CD5+CCRF-CEM tumor and then over time after multiple tumor rechallenges only the CD5 CAR-NK cells with DAP10 costimulation are able to control the tumor. CD19 CAR-NK cells and CD19/IL-15 CAR-NK cells were used as irrelevant CAR controls. 7C. Graph showing the cell confluence (%) over time which correlates with burden of live tumor cells. This shows that at the beginning all NK cells even non transduced (NT) NK cells are able to kill the CD5+CCRF-CEM tumor and then over time after multiple tumor rechallenges only the CD5 CAR-NK cells with DAP10 costimulation are able to control the tumor.

FIGS. 8A-8B. CD5 CAR-NK cells persist and can mount recall against tumor rechallenge in vivo. 8A. Schematic diagram showing the details of the experimental plan of the in vivo mouse model showing timing of irradiation, timing of CD5+CCRF-CEM tumor injection, timing of CD5 CAR-NK cells infusion and timing of rechallenge with CD5+CCRF-CEM tumor (transduced with GFP). 8B. FACS plots showing pre-rechallenge (left sided panels) and post-rechallenge flow cytometry data (right sided panels) showing human CD45+ gate and NK cell gate (CD56+ and GFP−). This shows that CD5 CAR-NK cells expand after tumor rechallenge and can mount a recall response against CD5+CCRF-CEM tumor.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### 1. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, such as that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides that are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “sample,” as used herein, generally refers to a biological sample. The sample may be taken from tissue or cells from an individual. In some examples, the sample may comprise, or be derived from, a tissue biopsy, blood (e.g., whole blood), blood plasma, extracellular fluid, dried blood spots, cultured cells, discarded tissue. The sample may have been isolated from the source prior to collection. Non-limiting examples include blood, cerebral spinal fluid, pleural fluid, amniotic fluid, lymph fluid, saliva, urine, stool, tears, sweat, or mucosal excretions, and other bodily fluids isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may be treated with RNases. The sample may contain tissues or cells that are intact, fragmented, or partially degraded.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

The present disclosure concerns methods and compositions directed to therapies for CD5-positive cancers, particularly utilizing adoptive cell therapy that targets CD5-positive cancer cells. In particular embodiments, genetically engineered mammalian immune cells of any kind (including at least human NK cells) are generated to target CD5-positive cancers. The disclosure encompasses a genetically engineered receptor of any kind (including a chimeric antigen receptor (CAR)) that is directed against CD5. In specific embodiments there are provided a number of novel expression constructs, including retroviral constructs, that express a CD5-targeting single chain variable fragment (scFv) used in a CAR and in some cases that also express one or more cytokines, such as IL-15, to support cell survival and proliferation.

Provided herein are designed CARs that target CD5, expressed on at least T-cell malignancies such as T-cell leukemia (T-ALL) and T-cell lymphoma. NK cells are especially advantageous to target CD5 for the immunotherapy of T-cell malignancies because, unlike normal T cells, they do not express CD5, which is a molecule expressed on all T-lymphoblastic leukemia cells as well as normal T-cells. Therefore, unlike CAR T cells, the CAR NK cells will not be targeted by CAR.CD5-mediated fratricide. Thus, the disclosure provides a number of novel retroviral constructs that express a single chain variable fragment (scFv) against CD5 and in some cases that also express IL-15 to support NK cell survival and proliferation. In a series of in vitro and in vivo studies, the inventors have confirmed the activity of CAR5/IL-15 transduced CB-NK cells against T-cell malignancies.

### I. Genetically Engineered Receptors

The immune cells of the present disclosure can be genetically engineered to express one or more antigen-binding receptors that target CD5, such as engineered CARs or, alternatively, engineered TCRs. For example, the immune cells may be immune cells that are modified to express a CAR and/or TCR having antigenic specificity for CD5. Other CARs and/or TCRs may be expressed by the same cells as the CD5 antigen receptor-expressing cells, and they may be directed to different antigens. In some aspects, the immune cells are engineered to express the CD5-specific CAR or CD5-specific TCR by knock-in of the CAR or TCR using CRISPR.

Suitable methods of modification of cells are known in the art. See, for instance, Sambrook and Ausubel, supra. For example, the cells may be transduced to express a CAR or TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen-targeting receptors (at least one of which is directed against CD5), and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

### A. Chimeric Antigen Receptors

In particular embodiments, a CD5-specific CAR is utilized that comprises at least: a) one or more intracellular signaling domains, b) a transmembrane domain, and c) an extracellular domain comprising at least one antigen binding region that targets, including specifically binds, CD5. In particular embodiments the antigen binding region is an antibody or functional fragment thereof, although in other cases the antigen binding region of the CAR is not an antibody or functional fragment thereof (such as a ligand for CD5). In some embodiments, the CD5-specific CAR binds only CD5, whereas in other cases the CAR as a single polypeptide is bispecific by comprising two or more antigen binding domains, one of which that binds CD5 and the other of which binds another, non-identical antigen.

In some embodiments, the engineered antigen receptors include CARs, including activating or stimulatory CARs, or costimulatory CARs (see WO2014/055668. The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric CAR construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

Certain embodiments of the present disclosure concern the use of nucleic acids, including nucleic acids encoding a CD5-specific CAR polypeptide, including in some cases a CAR that has been humanized to reduce immunogenicity (hCAR), comprising at least one intracellular signaling domain, a transmembrane domain, and an extracellular domain comprising one or more signaling motifs. In certain embodiments, the CD5-specific CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CD5 CAR nucleic acids may be human genes used to enhance cellular immunotherapy for human patients. In a specific embodiment, the disclosure includes a full-length CD5-specific CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is a CD5-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, CD5-specific CAR is constructed with specificity for CD5, such as CD5 being expressed on a diseased cell type. Thus, the CAR typically includes in its extracellular portion one or more CD5-binding molecules, such as one or more antigen-binding fragments, domains, antibody variable domains, and/or antibody molecules of any kind. An example of a human CD5 nucleic acid is at National Center for Biotechnology Information's GenBank® database at Accession No. NM_014207. An example of a human CD5 polypeptide is at GenBank® Accession No. NP_055022. One of skill in the art is able to generate antibodies, including scFvs against CD5 based on knowledge at least of the polypeptide and routine practices, although numerous anti-CD5 scFvs and monoclonal antibodies are already present in the art.

In some embodiments, the CD5-specific CAR includes an antigen-binding portion or portions of an antibody molecule, such as a single-chain antibody fragment (scFv) derived from the variable heavy (V_{H}) and variable light (V_{L}) chains of a monoclonal antibody (mAb). In specific embodiments, the antibody or functional fragment thereof is or is derived from H65 (Santa Cruz Biotechnology; Santa Cruz, Calif.); clone CRISI or clone 4C7 (Abnova Corporation; Walnut, Calif.); OX-19 (Santa Cruz Biotechnology; Santa Cruz, Calif.); Leu-1 (Becton-Dickinson; Mountain View, Calif.) and UCHT2 (Accurate Scientific; Westbury, N.Y.); 53-7.3 (Affymetrix; Santa Clara, Calif.); 4H8E6 (Life Technologies; Grand Island, N.Y.); T101; EP2952 (Abcam, Cambridge Mass.); or L17F12. In other embodiments, the antibody, e.g., scFv, is or is derived from anti-CD5 antibodies D-9, H-3, HK231, N-20, Y2/178, H-300, L17F12, CD5/54/F6, Q-20, or CC17 (all available from Santa Cruz Biotechnology, Dallas Tex.). The antibody may also be one that is generated de novo against CD5, and the scFv sequence may be obtained, or derived, from such de novo antibodies.

In certain embodiments, the anti-CD5 CAR comprises an extracellular domain that is or comprises a ligand for CD5. In specific embodiments, the anti-CD5 CAR comprises an extracellular domain that comprises CD72 (Lyb-2), gp40-80, gp150, gp200, IgVH framework region, IL-6, and fragments and mimetics thereof.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof, as it is found that introns stabilize the mRNA. Also, it may be further advantageous to use endogenous or exogenous non-coding regions to stabilize the mRNA.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex. The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, DAP12, DAP10, NKG2D, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, and so forth. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In some embodiments, the CD5 CAR nucleic acid comprises a sequence encoding other costimulatory receptors, such as a transmembrane domain and one or more intracellular signaling domains. In addition to a primary T cell activation signal, such as may be initiated by CD3ζ and/or FcεRIγ, an additional stimulatory signal for immune effector cell proliferation and effector function following engagement of the chimeric receptor with the target antigen may be utilized. For example, part or all of a human costimulatory receptor for enhanced activation of cells may be utilized that could help improve in vivo persistence and improve the therapeutic success of the adoptive immunotherapy. Examples include costimulatory domains from molecules such as DAP12, DAP10, NKG2D, CD2, CD28, CD27, 4-1BB, (CD137), OX40, ICOS, (CD278), CD30, HVEM, CD40, LFA-1 (CD11a/CD18), and/or ICAM-1, although in specific alternative embodiments any one of these listed may be excluded from use in the CAR.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the CD5-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

### B. Examples of Specific CAR Embodiments

In particular embodiments, specific CD5 CAR molecules are encompassed herein. In some cases, the CD5 binding domain of the CAR is a scFv, and any scFv that binds to CD5 and/or ligand that binds CD5 may be utilized herein. In cases wherein an anti-CD5 scFv is utilized in the extracellular domain of the CAR, the variable heavy chain and the variable light chain for the scFv may be in any order in N-terminal to C-terminal direction. For example, the variable heavy chain may be on the N-terminal side of the variable light chain, or vice versa. The scFv and/or ligand that binds CD5 in the CAR may or may not be codon optimized. In particular embodiments, a vector encodes a CD5-specific CAR and also encodes one or more other molecules. For example, a vector may encode a CD5-specific CAR and also may encode another protein of interest, such as another engineered antigen receptor, a suicide gene, and/or a particular cytokine.

On the same molecule, the CD5-specific CAR may comprise one or more antigen-specific extracellular domains, a specific hinge, a specific transmembrane domain, one or more specific costimulatory domains, and one or more specific activation signals. When more than one antigen-specific extracellular domain is utilized, such as for targeting two different antigens (one of which is CD5), there may be a linker between the two antigen-specific extracellular domains.

In particular embodiments of specific CAR molecules, a CAR may utilize DAP10, DAP12, 4-1BB, NKG2D, or other costimulatory domains (which may be referred to herein as an intracytoplasmic domain). In some cases, CD3zeta is utilized without any costimulatory domains. In particular embodiments of specific CAR molecules, a CAR may utilized any suitable transmembrane domain, such as from DAP12, DAP10,NKG2D or CD28.

In particular embodiments, there is an expression construct comprising sequence that encodes a particular CD5-specific engineered receptor. In particular embodiments, any CD5 CAR may comprise one of the following: (a) CD28 transmembrane domain (TM) and CD28 intracytoplasmic domain (ICD); (b) CD28 TM, CD28 ICD, and CD3zeta signaling domain; (c) CD28 TM and DAP12 ICD; (d) CD28 TM, DAP12 ICD, and CD3zeta signaling domain; (e) DAP12 TM and DAP12 ICD; (f) DAP12 TM, DAP12 ICD, and CD3zeta signaling domain; (g) CD28 TM and 4-1BB ICD; (h) CD28 TM, CD28 ICD, and 4-1BB ICD; (i) CD28 TM, CD28 ICD, 4-1BB ICD, and CD3zeta signaling domain; (j) CD28 TM and DAP10 ICD; (k) CD28TM, DAP10 ICD, and CD3zeta signaling domain; (l) DAP10 TM and DAP10 ICD; (m) DAP10 TM, DAP10 ICD, and CD3zeta signaling domain; (n) NKG2D ICD; (o) NKG2D TM; (p) NKG2D TM and NKG2D ICD; (q) NKG2D TM, NKG2D ICD, and CD3zeta signaling domain; (r) CD28 TM and NKG2D ICD; (s) NKG2D ICD, and CD3zeta signaling domain; (t) CD28 TM, NKG2D ICD, and CD3zeta signaling domain; or (u) CD28TM and CD3zeta in the absence of an ICD that is a costimulatory domain.

Examples of specific sequence embodiments are provided below.

### 1. Antigen-Specific Extracellular Domains

In specific embodiments, an anti-CD5 scFv (humanized) nucleotide sequence is utilized, as follows:

(SEQ ID NO: 1)
ATGGAGTTCGGTCTCAGTTGGCTGTTTTTGGTTGCTATCTTGAAGGGCG
TCCAATGCAGCCGGGACATCCAGATGACCCAGTCTCCCTCTAGCATGTC
AGCGAGTCTTGGTGATCGAGTGACGATTACCTGCAGAGCCTCTCAAGAT
ATAAACAGCTATCTTTCATGGTTCCAACAGAAGCCGGGGAAGTCCCCAA
AAACTCTCATATACAGGGCGAATCGACTCGTAGACGGTGTGCCTTCAAG
GTTTTCCGGGAGTGGTAGTGGCACAGATTACACACTTACAATCTCTTCA
TTGCAGTATGAGGATTTCGGGATCTACTACTGTCAACAGTACGACGAAT
CCCCATGGACGTTTGGGGGCGGGACCAAACTTGAGATAAAAGGGAGCAC
ATCTGGAAGTGGTAAACCTGGGTCAGGGGAGGGTTCCACAAAAGGACAA
ATTCAACTTGTCCAAAGCGGTCCTGGTCTTAAGAAGCCTGGAGGGTCTG
TCAGGATAAGTTGTGCGGCATCCGGCTACACCTTCACCAACTATGGGAT
GAACTGGGTGAAACAAGCGCCTGGGAAAGGTCTTCGATGGATGGGCTGG
ATTAATACCCACACTGGAGAGCCCACTTACGCTGATGATTTCAAAGGAC
GATTTACCTTCTCCTTGGATACTTCCAAGAGTACCGCGTACTTGCAAAT
CAACAGTCTCCGGGCTGAAGACACGGCCACATACTTCTGTACGCGGAGA
GGGTATGACTGGTATTTTGATGTGTGGGGTCAGGGAACAACCGTGACTG
TTTCAAGC

A translated scFv (humanized) (translated from SEQ ID NO:1) amino acid sequence is as follows:

(SEQ ID NO: 2)
MEFGLSWLFLVAILKGVQCSRDIQMTQSPSSMSASLGDRVTITCRASQD
INSYLSWFQQKPGKSPKTLIYRANRLVDGVPSRFSGSGSGTDYTLTISS
LQYEDFGIYYCQQYDESPWTFGGGTKLEIKGSTSGSGKPGSGEGSTKGQ
IQLVQSGPGLKKPGGSVRISCAASGYTFTNYGMNWVKQAPGKGLRWMGW
INTHTGEPTYADDFKGRFTFSLDTSKSTAYLQINSLRAEDTATYFCTRR
GYDWYFDVWGQGTTVTVSS

In specific embodiments, an anti-CD5 scFv [murine] nucleotide sequence is utilized, as follows:

(SEQ ID NO: 3)
ATGGAGTTCGGCTTGAGTTGGTTGTTCCTTGTGGCGATACTCAAAGGCG
TTCAATGTAGCCGAGACATAAAAATGACCCAGTCTCCGTCATCTATGTA
TGCAAGCCTCGGCGAGCGAGTGACCATCACGTGCAAGGCGAGTCAAGAT
ATAAACAGCTACTTGTCATGGTTCCAACAAAAACCAGGGAAATCACCTA
AGACCCTGATCTATAGAGCCAATCGCCTGGTTGACGGTGTCCCCTCCCG
CTTTAGCGGCTCCGGAAGCGGTCAAGATTACTCTCTCACAATTTCTTCC
TTGGATTATGAAGACATGGGGATCTACTATTGTCAACAGTATGACGAAT
CCCCGTGGACTTTCGGTGGCGGTACCAAATTGGAAATAAAGGGCTCTAC
AAGCGGCTCAGGAAAACCTGGATCAGGCGAAGGGTCTACGAAGGGCCAG
ATACAACTCGTTCAAAGTGGGCCAGAACTCAAAAAACCGGGAGAAACAG
TGAAAATTTCTTGTAAGGCATCAGGATACACATTCACAAACTACGGGAT
GAATTGGGTCAAACAAGCACCCGGAAAGGGGCTGCGCTGGATGGGGTGG
ATCAACACACATACTGGGGAACCTACTTACGCAGACGATTTCAAGGGCA
GATTCGCCTTTTCTTTGGAGACCTCCGCCTCTACTGCATACTTGCAGAT
AAACAACCTGAAGAATGAAGATACCGCCACCTACTTCTGTACGCGCAGG
GGCTACGATTGGTATTTTGATGTATGGGGGGCAGGCACCACTGTTACTG
TGTCAAGC

A translated scFv (murine) (translated from SEQ ID NO:3) amino acid sequence is as follows:

(SEQ ID NO: 4)
MEFGLSWLFLVAILKGVQCSRDIKMTQSPSSMYASLGERVTITCKASQD
INSYLSWFQQKPGKSPKTLIYRANRLVDGVPSRFSGSGSGQDYSLTISS
LDYEDMGIYYCQQYDESPWTFGGGTKLEIKGSTSGSGKPGSGEGSTKGQ
IQLVQSGPELKKPGETVKISCKASGYTFTNYGMNWVKQAPGKGLRWMGW
INTHTGEPTYADDFKGRFAFSLETSASTAYLQINNLKNEDTATYFCTRR
GYDWYFDVWGAGTTVTVSS

### 2. Transmembrane Domains

Any suitable transmembrane domain may be utilized in the CD5-specific CAR. Examples include at least from DAP10, DAP12, CD28, NKG2D, CD3 epsilon, CD4, CD5, CD8, CD9, CD16, CD22, CD28, CD33, CD37, CD45, CD64, CD80, CD86, CD134, CD137, CD154, from a T-cell receptor a or b chain, a CD3 zeta chain, ICOS, functional derivatives thereof, and combinations thereof. In specific cases, the transmembrane domain from DAP10, DAP12, CD28, or NKG2D is utilized. Examples of particular transmembrane domain sequences may be used, as follows:

### 4-1BB Transmembrane Domain Nucleotide Sequence:

(SEQ ID NO: 5)
ATCATCTCCTTCTTTCTTGCGCTGACGTCGACTGCGTTGCTCTTCCTGC
TGTTCTTCCTCACGCTCCGTTTCTCTGTTGTT

### 4-1BB Transmembrane Domain Amino Acid Sequence:

(SEQ ID NO: 6)
IISFFLALTSTALLFLLFFLTLRFSVV

### CD28 Transmembrane Domain Nucleotide Sequence:

(SEQ ID NO: 7)
TTTTGGGTGCTGGTGGTGGTTGGTGGAGTCCTGGCTTGCTATAGCTTGC
TAGTAACAGTGGCCTTTATTATTTTCTGGGTG

### CD28 Transmembrane Domain Amino Acid Sequence:

(SEQ ID NO: 8)
FWVLVVVGGVLACYSLLVTVAFIIFWV

### DAP10 Transmembrane Domain Nucleotide Sequence:

(SEQ ID NO: 9)
CTCCTGGCAGGCCTCGTGGCTGCTGATGCGGTGGCATCGCTGCTCATCG
TGGGGGCGGTGTTC

### DAP10 Transmembrane Domain Amino Acid Sequence:

(SEQ ID NO: 10)
LLAGLVAADAVASLLIVGAVF

### DAP12 Transmembrane Domain Nucleotide Sequence:

(SEQ ID NO: 11)
GGCGTGCTGGCAGGGATCGTGATGGGAGACCTGGTGCTGACAGTGCTCAT
TGCCCTGGCCGTG

### DAP12 Transmembrane Domain Amino Acid Sequence:

(SEQ ID NO: 12)
GVLAGIVMGDLVLTVLIALAV

### NKG2D Transmembrane Domain Nucleotide Sequence:

(SEQ ID NO: 13)
GCGGTGATGATTATTTTTCGCATTGGCATGGCGGTGGCGATTTTTTGCTG
CTTTTTTTTTCCG

### NKG2D Transmembrane Domain Amino Acid Sequence:

(SEQ ID NO: 14)
AVMIIFRIGMAVAIFCCFFFP

### 3. Intracytoplasmic Domains (ICD)

One or more intracytoplasmic domains (that may also be referred to herein as costimulatory domains, in appropriate cases) may or may not be utilized in specific anti-CD5 CARs of the disclosure. Specific examples include ICDs from 4-1BB, NKG2D, DAP10, DAP12, B7-1/CD80, CD28, 4-1BBL, B7-2/CD86, CTLA-4, B7-H1/PD-L1, ICOS, B7-H2, PD-1, B7-H3, PD-L2, B7-H4, PDCD6, BTLA; or a combination thereof.

Examples of particular ICD sequences may be used in the CAR, as follows:

### 4-1BB Intracytoplasmic Domain Nucleotide Sequence:

(SEQ ID NO: 15)
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG

### 4-1BB Intracytoplasmic Domain Amino Acid Sequence:

(SEQ ID NO: 16)
KRGRKKLLYIFKQPFMRPVQTTQEEDGCSCRFPEEEEGGCEL

### DAP10 Intracytoplasmic Domain Nucleotide Sequence:

(SEQ ID NO: 17)
CTTTGCGCACGCCCACGCCGCAGCCCCGCCCAAGAAGATGGCAAAGTCT
ACATCAACATGCCAGGCAGGGGC

### DAP10 Intracytoplasmic Domain Amino Acid Sequence:

(SEQ ID NO: 18)
LCARPRRSPAQEDGKVYINMPGRG

### DAP12 Intracytoplasmic Domain Nucleotide Sequence:

(SEQ ID NO: 19)
TACTTCCTGGGCCGGCTGGTCCCTCGGGGGCGAGGGGCTGCGGAGGCAGC
GACCCGGAAACAGCGTATCACTGAGACCGAGTCGCCTTATCAGGAGCTCC
AGGGTCAGAGGTCGGATGTCTACAGCGACCTCAACACACAGAGGCCGTAT
TACAAA

### DAP12 Intracytoplasmic Domain Amino Acid Sequence:

(SEQ ID NO: 20)
YFLGRLVPRGRGAAEAATRKQRITETESPYQELQGQRSDVYSDLNTQRP
YYK

### NKG2D Intracytoplasmic Domain Nucleotide Sequence:

(SEQ ID NO: 21)
AGCGCGAACGAACGCTGCAAAAGCAAAGTGGTGCCGTGCCGCCAGAAACA
GTGGCGCACCAGCTTTGATAGCAAAAAACTGGATCTGAACTATAACCATT
TTGAAAGCATGGAATGGAGCCATCGCAGCCGCCGCGGCCGCATTTGGGGC
ATG

### NKG2D Intracytoplasmic Domain Amino Acid Sequence:

(SEQ ID NO: 22)
SANERCKSKVVPCRQKQWRTSFDSKKLDLNYNHFESMEWSHRSRRGRI
WGM

### 4. Hinge

In some embodiments of the CARs, there is a hinge region between the one or more extracellular antigen binding domains and the transmembrane domain. In specific embodiments, the hinge is of a particular length, such as 10-20, 10-15, 11-20, 11-15, 12-20, 12-15, or 15-20 amino acids in length, for example. The hinge may be any suitable hinge and includes a hinge from IgG, or CD28, in some cases. In specific embodiments, the hinge is a small flexible polypeptide that connects CH2-CH3 and CH1 domains of IgG Fc. For example, one may utilize CH2-CH3 hinge (part or all) from various IgG subclasses (IgG1-4, either modified or not). However, in some cases the entire CH2-CH3 hinge is not utilized but instead a portion of the hinge is used (such as CH3 by itself or part of CH3 by itself). In particular embodiments, the CH2-CH3 hinge derived from IgG1 is utilized, and in some cases the entire CH2-CH3 hinge is used (all 229 amino acids), only the CH3 hinge (119 amino acids) is used, or a short hinge (12 amino acids) is used.

In specific cases, one can modify the identity or length of the spacer and/or hinge to optimize efficiency of the CAR. See, for example, Hudecek et al. (2014) and Jonnalagadda et al. (2015) In specific embodiments, the CD5 CAR utilizes IgG4 hinge+CH3 or utilizes CD8a stalk, for example.

Thus, in specific embodiments the IgG hinge region that is utilized is typically IgG1 or IgG4, and in some cases the CAR comprises the CH2-CH3 domain of IgG Fc. The use of the IgG Fc domain can provide flexibility to the CAR, has low immunogenicity, facilitates detection of CAR expression using anti-Fc reagents, and allows removal of one or more CH2 or CH3 modules to accommodate different spacer lengths. However, in one embodiment mutations in certain spacers to avoid FcγR binding may improve CAR+ T cell engraftment and antitumor efficacy to avoid binding of soluble and cell surface Fc gamma receptors, for example, yet maintain the activity to mediate antigen-specific lysis. For example, one can employ IgG4-Fc spacers that have either been modified in the CH2 region. For example, the CH2 region may be mutated, including point mutations and/or deletions. Specific modifications have been demonstrated at two sites (L235E; N297Q) within the CH2 region and/or incorporate a CH2 deletion (Jonnalagadda et al, 2015). In specific embodiments, one may employ the IgG4 hinge-CH2-CH3 domain (229 aa in length) or only the hinge domain (12 aa in length) (Hudececk et al., 2015).

In specific embodiments, the hinge is from IgG, CD28, CD-8 alpha, 4-1BB, 0X40, CD3-zeta, T cell receptor a or b chain, a CD3 zeta chain, CD28, CD3e, CD45, CD4, CD5, CD8, CD9, CD16, CD22, CD33, CD37, CD64, CD80, CD86, CD134, CD137, ICOS, or CD154.

Examples of specific sequences of hinges that may be utilized include at least the following:

### IgG Hinge Nucleotide Sequence:

(SEQ ID NO: 23)
GTACGGTCACTGTCTCTTCACAGGATCCCGCCGAGCCCAAATCTCCTGAC
AAAACTCACACATGCCCACCGTGCCCAGCACCTGAACTCCTGGGGGGACC
GTCAGTCTTCCTCTTCCCCCCAAAACCCAAGGACACCCTCATGATCTCCC
GGACCCCTGAGGTCACATGCGTGGTGGTGGACGTGAGCCACGAAGACCCT
GAGGTCAAGTTCAACTGGTACGTGGACGGCGTGGAGGTGCATAATGCCAA
GACAAAGCCGCGGGAGGAGCAGTACAACAGCACGTACCGTGTGGTCAGCG
TCCTCACCGTCCTGCACCAGGACTGGCTGAATGGCAAGGAGTACAAGTGC
AAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCC
GGGATGAGCTGACCAAGAACCAGGTCAGCCTGACCTGCCTGGTCAAAGGC
TTCTATCCCAGCGACATCGCCGTGGAGTGGGAGAGCAATGGGCAACCGGA
GAACAACTACAAGACCACGCCTCCCGTGCTGGACTCCGACGGCTCCTTCT
TCCTCTACAGCAAGCTCACCGTGGACAAGAGCAGGTGGCAGCAGGGGAAC
GTCTTCTCATGCTCCGTGATGCATGAGGCTCTGCACAACCACTACACGCA
GAAGAGCCTCTCCCTGTCTCCGGGTAAAAAAGATCCCAAATT

### IgG Hinge Amino Acid Sequence:

(SEQ ID NO: 24)
TVTVSSQDPAEPKSPDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEALHNHYTQKSLSLSPGKKDPK

### CD28 Hinge Nucleotide Sequence:

(SEQ ID NO: 25)
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCC

### CD28 Hinge Amino Acid Sequence:

(SEQ ID NO: 26)
IEVMYPPPYLDNEKSNGTIIHVKGKHLCPSPLFPGPSKP

### 5. Signal Activation Domain

In specific cases, the anti-CD5 CAR molecules comprise a signal activation domain, such as from CD3zeta or FcεRIγ, for example.

One example of a CD3 zeta nucleotide sequence that may be utilized is as follows:

(SEQ ID NO: 27)
CGCGTgtGAAGTTCAGCAGGAGCGCAGACGCCCCCGCGTACCAGC
AGGGCCAGAACCAGCTCTATAACGAGCTCAATCTAGGACGAAGAG
AGGAGTACGATGTTTTGGACAAAAGACGTGGCCGGGACCCTGAGA
TGGGGGGAAAGCCGAGAAGGAAGAACCCTCAGGAAGGCCTGTACA
ATGAACTGCAGAAAGATAAGATGGCGGAGGCCTACAGTGAGATTG
GGATGAAAGGCGAGCGCCGGAGGGGCAAGGGGCACGATGGCCTTT
ACCAGGGTCTCAGTACAGCCACCAAGGACACCTACGACGCCCTTC
ACATGCAGGCCCTGCCCCCTCGCGGAC

One example of a CD3 zeta amino acid sequence that may be utilized is as follows:

(SEQ ID NO: 28)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPRG

### 6. Other Proteins

In some embodiments, one or more other proteins are utilized with the anti-CD5 CAR. The one or more other proteins may be utilized for any reason, including to facilitate efficacy of the CAR itself and/or of any kind of cells expressing the CAR. In some cases, the other protein facilitates treatment of an individual receiving cells expressing the CAR as therapy, whether or not the other protein(s) directly or indirectly impact activity of the CAR or the cells. In some cases, the other protein is a suicide gene, one or more cytokines, or both. In specific embodiments, one or more other proteins are produced from a vector and ultimately are produced as two separate polypeptides. For example, the anti-CD5 CAR and the other protein(s) may be separated by a 2A sequence or by an IRES, for example.

In specific embodiments, a cytokine such as IL-15 is utilized in conjunction with the anti-CD5 CAR.

One example of an IL-15 nucleotide sequence is as follows:

### IL-15 Nucleotide Sequence:

(SEQ ID NO: 29)
GCATTAGCAAGCCCCACCTGCGGAGCATCAGCATCCAGTGCTACC
TGTGCCTGCTGCTGAACAGCCACTTCCTGACCGAGGCCGGCATCC
ACGTGTTCATCCTGGGCTGCTTCAGCGCCGGACTGCCCAAGACCG
AGGCCAACTGGGTGAACGTGATCAGCGACCTGAAGAAGATCGAGG
ACCTGATCCAGAGCATGCACATCGACGCCACCCTGTACACCGAGA
GCGACGTGCACCCCAGCTGCAAGGTGACCGCCATGAAGTGCTTTC
TGCTGGAACTGCAGGTGATCAGCCTGGAAAGCGGCGACGCCAGCA
TCCACGACACCGTGGAGAACCTGATCATCCTGGCCAACAACAGCC
TGAGCAGCAACGGCAACGTGACCGAGAGCGGCTGCAAAGAGTGCG
AGGAACTGGAAGAGAAGAACATCAAAGAGTTTCTGCAGAGCTTCG
TGCACATCGTGCAGATGTTCATCAACACCAGCTGACAATT

### IL-15 Amino Acid Sequence:

(SEQ ID NO: 30)
ISKPHLRSISIQCYLCLLLNSHFLTEAGIHVFILGCFSAGLPKTE
ANWVNVISDLKKIEDLIQSMHIDATLYTESDVHPSCKVTAMKCFL
LELQVISLESGDASIHDTVENLIILANNSLSSNGNVTESGCKECE
ELEEKNIKEFLQSFVHIVQMFINTS

In cases wherein the CAR and another protein in the same vector are intended to be produced into two different polypeptides, a specific 2A sequence may be utilized.

In one example, an E2A nucleotide sequence is utilized as follows:

(SEQ ID NO: 31)
CAGTGTACTAATTATGCTCTCTTGAAATTGGCTGGAGATGTTGAG
AGCAATCCCGGGCCC

E2A amino acid sequence may be utilized as follows:

(SEQ ID NO: 32)
QCTNYALLKLAGDVESNPGP

Other 2A examples may be utilized and are as follows:

T2A:
(SEQ ID NO: 49)
EGRGSLLTCGDVEENPGP
P2A:
(SEQ ID NO: 50)
ATNFSLLKQAGDVEENPGP
F2A:
(SEQ ID NO: 51)
VKQTLNFDLLKLAGDVESNPGP

The disclosure also encompasses specific CAR molecules, including for expression in any type of immune effector cells.

In one example, an anti-CD5 CAR comprising an IgG1 hinge, CD28 transmembrane domain, and CD3zeta is utilized. In a vector, the CAR may be expressed with IL-15, such as may be separated from the CAR by a 2A sequence. In a specific example, such a CAR and IL-15 construct may have the following nucleotide sequence:

### CD5-IgG1-CD28TMD-CD3zeta-2A-IL15

(SEQ ID NO: 33)
ATGCTCGAGATGGAGTTCGGCTTGAGTTGGTTGTTCCTTGTGGCG
ATACTCAAAGGCGTTCAATGTAGCCGAGACATAAAAATGACCCAG
TCTCCGTCATCTATGTATGCAAGCCTCGGCGAGCGAGTGACCATC
ACGTGCAAGGCGAGTCAAGATATAAACAGCTACTTGTCATGGTTC
CAACAAAAACCAGGGAAATCACCTAAGACCCTGATCTATAGAGCC
AATCGCCTGGTTGACGGTGTCCCCTCCCGCTTTAGCGGCTCCGGA
AGCGGTCAAGATTACTCTCTCACAATTTCTTCCTTGGATTATGAA
GACATGGGGATCTACTATTGTCAACAGTATGACGAATCCCCGTGG
ACTTTCGGTGGCGGTACCAAATTGGAAATAAAGGGCTCTACAAGC
GGCTCAGGAAAACCTGGATCAGGCGAAGGGTCTACGAAGGGCCAG
ATACAACTCGTTCAAAGTGGGCCAGAACTCAAAAAACCGGGAGAA
ACAGTGAAAATTTCTTGTAAGGCATCAGGATACACATTCACAAAC
TACGGGATGAATTGGGTCAAACAAGCACCCGGAAAGGGGCTGCGC
TGGATGGGGTGGATCAACACACATACTGGGGAACCTACTTACGCA
GACGATTTCAAGGGCAGATTCGCCTTTTCTTTGGAGACCTCCGCC
TCTACTGCATACTTGCAGATAAACAACCTGAAGAATGAAGATACC
GCCACCTACTTCTGTACGCGCAGGGGCTACGATTGGTATTTTGAT
GTATGGGGGGCAGGCACCACTGTTACTGTGTCAAGCCGTACGGTC
ACTGTCTCTTCACAGGATCCCGCCGAGCCCAAATCTCCTGACAAA
ACTCACACATGCCCACCGTGCCCAGCACCTGAACTCCTGGGGGGA
CCGTCAGTCTTCCTCTTCCCCCCAAAACCCAAGGACACCCTCATG
ATCTCCCGGACCCCTGAGGTCACATGCGTGGTGGTGGACGTGAGC
CACGAAGACCCTGAGGTCAAGTTCAACTGGTACGTGGACGGCGTG
GAGGTGCATAATGCCAAGACAAAGCCGCGGGAGGAGCAGTACAAC
AGCACGTACCGTGTGGTCAGCGTCCTCACCGTCCTGCACCAGGAC
TGGCTGAATGGCAAGGAGTACAAGTGCAAGGTCTCCAACAAAGCC
CTCCCAGCCCCCATCGAGAAAACCATCTCCAAAGCCAAAGGGCAG
CCCCGAGAACCACAGGTGTACACCCTGCCCCCATCCCGGGATGAG
CTGACCAAGAACCAGGTCAGCCTGACCTGCCTGGTCAAAGGCTTC
TATCCCAGCGACATCGCCGTGGAGTGGGAGAGCAATGGGCAACCG
GAGAACAACTACAAGACCACGCCTCCCGTGCTGGACTCCGACGGC
TCCTTCTTCCTCTACAGCAAGCTCACCGTGGACAAGAGCAGGTGG
CAGCAGGGGAACGTCTTCTCATGCTCCGTGATGCATGAGGCTCTG
CACAACCACTACACGCAGAAGAGCCTCTCCCTGTCTCCGGGTAAA
AAAGATCCCAAATTTTGGTTTTGGGTGCTGGTGGTGGTTGGTGGA
GTCCTGGCTTGCTATAGCTTGCTAGTAACAGTGGCCTTTATTATT
TTCTGGGTGACGCGTgtGAAGTTCAGCAGGAGCGCAGACGCCCCC
GCGTACCAGCAGGGCCAGAACCAGCTCTATAACGAGCTCAATCTA
GGACGAAGAGAGGAGTACGATGTTTTGGACAAAAGACGTGGCCGG
GACCCTGAGATGGGGGGAAAGCCGAGAAGGAAGAACCCTCAGGAA
GGCCTGTACAATGAACTGCAGAAAGATAAGATGGCGGAGGCCTAC
AGTGAGATTGGGATGAAAGGCGAGCGCCGGAGGGGCAAGGGGCAC
GATGGCCTTTACCAGGGTCTCAGTACAGCCACCAAGGACACCTAC
GACGCCCTTCACATGCAGGCCCTGCCCCCTCGCGGACCGCAGTGT
ACTAATTATGCTCTCTTGAAATTGGCTGGAGATGTTGAGAGCAAT
CCCGGGCCCATGCGCATTAGCAAGCCCCACCTGCGGAGCATCAGC
ATCCAGTGCTACCTGTGCCTGCTGCTGAACAGCCACTTCCTGACC
GAGGCCGGCATCCACGTGTTCATCCTGGGCTGCTTCAGCGCCGGA
CTGCCCAAGACCGAGGCCAACTGGGTGAACGTGATCAGCGACCTG
AAGAAGATCGAGGACCTGATCCAGAGCATGCACATCGACGCCACC
CTGTACACCGAGAGCGACGTGCACCCCAGCTGCAAGGTGACCGCC
ATGAAGTGCTTTCTGCTGGAACTGCAGGTGATCAGCCTGGAAAGC
GGCGACGCCAGCATCCACGACACCGTGGAGAACCTGATCATCCTG
GCCAACAACAGCCTGAGCAGCAACGGCAACGTGACCGAGAGCGGC
TGCAAAGAGTGCGAGGAACTGGAAGAGAAGAACATCAAAGAGTTT
CTGCAGAGCTTCGTGCACATCGTGCAGATGTTCATCAACACCAGC
TGA

A corresponding amino acid sequence for CD5-IgG1-CD28TMD-CD3zeta-2A-IL 15 is as follows:

(SEQ ID NO: 34)
MLEMEFGLSWLFLVAILKGVQCSRDIKMTQSPSSMYASLGERVTI
TCKASQDINSYLSWFQQKPGKSPKTLIYRANRLVDGVPSRFSGSG
SGQDYSLTISSLDYEDMGIYYCQQYDESPWTFGGGTKLEIKGSTS
GSGKPGSGEGSTKGQIQLVQSGPELKKPGETVKISCKASGYTFTN
YGMNWVKQAPGKGLRWMGWINTHTGEPTYADDFKGRFAFSLETSA
STAYLQINNLKNEDTATYFCTRRGYDWYFDVWGAGTTVTVSSRTV
TVSSQDPAEPKSPDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGKKDPKFWFWVLVVVGGVLACYSLLVTVAFII
FWVTRVKFSRSADAPAYQQGQNQLYNELNLGRREEYDVLDKRRGR
DPEMGGKPRRKNPQEGLYNELQKDKMAEAYSEIGMKGERRRGKGH
DGLYQGLSTATKDTYDALHMQALPPRGPQCTNYALLKLAGDVESN
PGPMRISKPHLRSISIQCYLCLLLNSHFLTEAGIHVFILGCFSAG
LPKTEANWVNVISDLKKIEDLIQSMHIDATLYTESDVHPSCKVTA
MKCFLLELQVISLESGDASIHDTVENLIILANNSLSSNGNVTESG
CKECEELEEKNIKEFLQSFVHIVQMFINTS

In one example, an anti-CD5 CAR comprising an IgG1 hinge, DAP10 transmembrane domain and DAP10 intracytoplasmic domain, and CD3zeta is utilized. In a vector, the CAR may be expressed with IL-15, such as may be separated from the CAR by a 2A sequence. In a specific example, such a CAR and IL-15 construct may have the following nucleotide sequence:

### CD5-IgG1-DAP10TMD-DAP10 ICD.CD3zeta-2A-IL15

(SEQ ID NO: 35)
ATGCTCGAGATGGAGTTCGGCTTGAGTTGGTTGTTCCTTGTGGCG
ATACTCAAAGGCGTTCAATGTAGCCGAGACATAAAAATGACCCAG
TCTCCGTCATCTATGTATGCAAGCCTCGGCGAGCGAGTGACCATC
ACGTGCAAGGCGAGTCAAGATATAAACAGCTACTTGTCATGGTTC
CAACAAAAACCAGGGAAATCACCTAAGACCCTGATCTATAGAGCC
AATCGCCTGGTTGACGGTGTCCCCTCCCGCTTTAGCGGCTCCGGA
AGCGGTCAAGATTACTCTCTCACAATTTCTTCCTTGGATTATGAA
GACATGGGGATCTACTATTGTCAACAGTATGACGAATCCCCGTGG
ACTTTCGGTGGCGGTACCAAATTGGAAATAAAGGGCTCTACAAGC
GGCTCAGGAAAACCTGGATCAGGCGAAGGGTCTACGAAGGGCCAG
ATACAACTCGTTCAAAGTGGGCCAGAACTCAAAAAACCGGGAGAA
ACAGTGAAAATTTCTTGTAAGGCATCAGGATACACATTCACAAAC
TACGGGATGAATTGGGTCAAACAAGCACCCGGAAAGGGGCTGCGC
TGGATGGGGTGGATCAACACACATACTGGGGAACCTACTTACGCA
GACGATTTCAAGGGCAGATTCGCCTTTTCTTTGGAGACCTCCGCC
TCTACTGCATACTTGCAGATAAACAACCTGAAGAATGAAGATACC
GCCACCTACTTCTGTACGCGCAGGGGCTACGATTGGTATTTTGAT
GTATGGGGGGCAGGCACCACTGTTACTGTGTCAAGCCGTACGGTC
ACTGTCTCTTCACAGGATCCCGCCGAGCCCAAATCTCCTGACAAA
ACTCACACATGCCCACCGTGCCCAGCACCTGAACTCCTGGGGGGA
CCGTCAGTCTTCCTCTTCCCCCCAAAACCCAAGGACACCCTCATG
ATCTCCCGGACCCCTGAGGTCACATGCGTGGTGGTGGACGTGAGC
CACGAAGACCCTGAGGTCAAGTTCAACTGGTACGTGGACGGCGTG
GAGGTGCATAATGCCAAGACAAAGCCGCGGGAGGAGCAGTACAAC
AGCACGTACCGTGTGGTCAGCGTCCTCACCGTCCTGCACCAGGAC
TGGCTGAATGGCAAGGAGTACAAGTGCAAGGTCTCCAACAAAGCC
CTCCCAGCCCCCATCGAGAAAACCATCTCCAAAGCCAAAGGGCAG
CCCCGAGAACCACAGGTGTACACCCTGCCCCCATCCCGGGATGAG
CTGACCAAGAACCAGGTCAGCCTGACCTGCCTGGTCAAAGGCTTC
TATCCCAGCGACATCGCCGTGGAGTGGGAGAGCAATGGGCAACCG
GAGAACAACTACAAGACCACGCCTCCCGTGCTGGACTCCGACGGC
TCCTTCTTCCTCTACAGCAAGCTCACCGTGGACAAGAGCAGGTGG
CAGCAGGGGAACGTCTTCTCATGCTCCGTGATGCATGAGGCTCTG
CACAACCACTACACGCAGAAGAGCCTCTCCCTGTCTCCGGGTAAA
AAAGATCCCAAATTTTGGCTCCTGGCAGGCCTCGTGGCTGCTGAT
GCGGTGGCATCGCTGCTCATCGTGGGGGCGGTGTTCCTTTGCGCA
CGCCCACGCCGCAGCCCCGCCCAAGAAGATGGCAAAGTCTACATC
AACATGCCAGGCAGGGGCACGCGTgtGAAGTTCAGCAGGAGCGCA
GACGCCCCCGCGTACCAGCAGGGCCAGAACCAGCTCTATAACGAG
CTCAATCTAGGACGAAGAGAGGAGTACGATGTTTTGGACAAAAGA
CGTGGCCGGGACCCTGAGATGGGGGGAAAGCCGAGAAGGAAGAAC
CCTCAGGAAGGCCTGTACAATGAACTGCAGAAAGATAAGATGGCG
GAGGCCTACAGTGAGATTGGGATGAAAGGCGAGCGCCGGAGGGGC
AAGGGGCACGATGGCCTTTACCAGGGTCTCAGTACAGCCACCAAG
GACACCTACGACGCCCTTCACATGCAGGCCCTGCCCCCTCGCGGA
CCGCAGTGTACTAATTATGCTCTCTTGAAATTGGCTGGAGATGTT
GAGAGCAATCCCGGGCCCATGCGCATTAGCAAGCCCCACCTGCGG
AGCATCAGCATCCAGTGCTACCTGTGCCTGCTGCTGAACAGCCAC
TTCCTGACCGAGGCCGGCATCCACGTGTTCATCCTGGGCTGCTTC
AGCGCCGGACTGCCCAAGACCGAGGCCAACTGGGTGAACGTGATC
AGCGACCTGAAGAAGATCGAGGACCTGATCCAGAGCATGCACATC
GACGCCACCCTGTACACCGAGAGCGACGTGCACCCCAGCTGCAAG
GTGACCGCCATGAAGTGCTTTCTGCTGGAACTGCAGGTGATCAGC
CTGGAAAGCGGCGACGCCAGCATCCACGACACCGTGGAGAACCTG
ATCATCCTGGCCAACAACAGCCTGAGCAGCAACGGCAACGTGACC
GAGAGCGGCTGCAAAGAGTGCGAGGAACTGGAAGAGAAGAACATC
AAAGAGTTTCTGCAGAGCTTCGTGCACATCGTGCAGATGTTCATC
AACACCAGCTGA

A corresponding amino acid sequence for CD5-IgG1-DAP10TMD-DAP10 ICD.CD3zeta-2A-IL15 is as follows:

(SEQ ID NO: 36)
MLEMEFGLSWLFLVAILKGVQCSRDIKMTQSPSSMYASLGERVTI
TCKASQDINSYLSWFQQKPGKSPKTLIYRANRLVDGVPSRFSGSG
SGQDYSLTISSLDYEDMGIYYCQQYDESPWTFGGGTKLEIKGSTS
GSGKPGSGEGSTKGQIQLVQSGPELKKPGETVKISCKASGYTFTN
YGMNWVKQAPGKGLRWMGWINTHTGEPTYADDFKGRFAFSLETSA
STAYLQINNLKNEDTATYFCTRRGYDWYFDVWGAGTTVTVSSRTV
TVSSQDPAEPKSPDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGKKDPKFWLLAGLVAADAVASLLIVGAVFLCA
RPRRSPAQEDGKVYINMPGRGTRVKFSRSADAPAYQQGQNQLYNE
LNLGRREEYDVLDKRRGRDPEMGGKPRRKNPQEGLYNELQKDKMA
EAYSEIGMKGERRRGKGHDGLYQGLSTATKDTYDALHMQALPPRG
PQCTNYALLKLAGDVESNPGPMRISKPHLRSISIQCYLCLLLNSH
FLTEAGIHVFILGCFSAGLPKTEANWVNVISDLKKIEDLIQSMHI
DATLYTESDVHPSCKVTAMKCFLLELQVISLESGDASIHDTVENL
IILANNSLSSNGNVTESGCKECEELEEKNIKEFLQSFVHIVQMFI
NTS

In one example, an anti-CD5 CAR comprising an IgG1 hinge, CD28 transmembrane domain and 4-1BB intracytoplasmic domain, and CD3zeta is utilized. In a vector, the CAR may be expressed with IL-15, such as may be separated from the CAR by a 2A sequence. In a specific example, such a CAR and IL-15 construct may have the following nucleotide sequence:

### CD5-IgG1-CD28TMD-41BB-CD3zeta-2A-IL15

(SEQ ID NO: 37)
ATGCTCGAGATGGAGTTCGGCTTGAGTTGGTTGTTCCTTGTGGCG
ATACTCAAAGGCGTTCAATGTAGCCGAGACATAAAAATGACCCAG
TCTCCGTCATCTATGTATGCAAGCCTCGGCGAGCGAGTGACCATC
ACGTGCAAGGCGAGTCAAGATATAAACAGCTACTTGTCATGGTTC
CAACAAAAACCAGGGAAATCACCTAAGACCCTGATCTATAGAGCC
AATCGCCTGGTTGACGGTGTCCCCTCCCGCTTTAGCGGCTCCGGA
AGCGGTCAAGATTACTCTCTCACAATTTCTTCCTTGGATTATGAA
GACATGGGGATCTACTATTGTCAACAGTATGACGAATCCCCGTGG
ACTTTCGGTGGCGGTACCAAATTGGAAATAAAGGGCTCTACAAGC
GGCTCAGGAAAACCTGGATCAGGCGAAGGGTCTACGAAGGGCCAG
ATACAACTCGTTCAAAGTGGGCCAGAACTCAAAAAACCGGGAGAA
ACAGTGAAAATTTCTTGTAAGGCATCAGGATACACATTCACAAAC
TACGGGATGAATTGGGTCAAACAAGCACCCGGAAAGGGGCTGCGC
TGGATGGGGTGGATCAACACACATACTGGGGAACCTACTTACGCA
GACGATTTCAAGGGCAGATTCGCCTTTTCTTTGGAGACCTCCGCC
TCTACTGCATACTTGCAGATAAACAACCTGAAGAATGAAGATACC
GCCACCTACTTCTGTACGCGCAGGGGCTACGATTGGTATTTTGAT
GTATGGGGGGCAGGCACCACTGTTACTGTGTCAAGCCGTACGGTC
ACTGTCTCTTCACAGGATCCCGCCGAGCCCAAATCTCCTGACAAA
ACTCACACATGCCCACCGTGCCCAGCACCTGAACTCCTGGGGGGA
CCGTCAGTCTTCCTCTTCCCCCCAAAACCCAAGGACACCCTCATG
ATCTCCCGGACCCCTGAGGTCACATGCGTGGTGGTGGACGTGAGC
CACGAAGACCCTGAGGTCAAGTTCAACTGGTACGTGGACGGCGTG
GAGGTGCATAATGCCAAGACAAAGCCGCGGGAGGAGCAGTACAAC
AGCACGTACCGTGTGGTCAGCGTCCTCACCGTCCTGCACCAGGAC
TGGCTGAATGGCAAGGAGTACAAGTGCAAGGTCTCCAACAAAGCC
CTCCCAGCCCCCATCGAGAAAACCATCTCCAAAGCCAAAGGGCAG
CCCCGAGAACCACAGGTGTACACCCTGCCCCCATCCCGGGATGAG
CTGACCAAGAACCAGGTCAGCCTGACCTGCCTGGTCAAAGGCTTC
TATCCCAGCGACATCGCCGTGGAGTGGGAGAGCAATGGGCAACCG
GAGAACAACTACAAGACCACGCCTCCCGTGCTGGACTCCGACGGC
TCCTTCTTCCTCTACAGCAAGCTCACCGTGGACAAGAGCAGGTGG
CAGCAGGGGAACGTCTTCTCATGCTCCGTGATGCATGAGGCTCTG
CACAACCACTACACGCAGAAGAGCCTCTCCCTGTCTCCGGGTAAA
AAAGATCCCAAATTTTGGTTTTGGGTGCTGGTGGTGGTTGGTGGA
GTCCTGGCTTGCTATAGCTTGCTAGTAACAGTGGCCTTTATTATT
TTCTGGGTGAAACGGGGCAGAAAGAAACTCCTGTATATATTCAAA
CAACCATTTATGAGACCAGTACAAACTACTCAAGAGGAAGATGGC
TGTAGCTGCCGATTTCCAGAAGAAGAAGAAGGAGGATGTGAACTG
ACGCGTgtGAAGTTCAGCAGGAGCGCAGACGCCCCCGCGTACCAG
CAGGGCCAGAACCAGCTCTATAACGAGCTCAATCTAGGACGAAGA
GAGGAGTACGATGTTTTGGACAAAAGACGTGGCCGGGACCCTGAG
ATGGGGGGAAAGCCGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCGGACCGCAGTGTACTAATTAT
GCTCTCTTGAAATTGGCTGGAGATGTTGAGAGCAATCCCGGGCCC
ATGCGCATTAGCAAGCCCCACCTGCGGAGCATCAGCATCCAGTGC
TACCTGTGCCTGCTGCTGAACAGCCACTTCCTGACCGAGGCCGGC
ATCCACGTGTTCATCCTGGGCTGCTTCAGCGCCGGACTGCCCAAG
ACCGAGGCCAACTGGGTGAACGTGATCAGCGACCTGAAGAAGATC
GAGGACCTGATCCAGAGCATGCACATCGACGCCACCCTGTACACC
GAGAGCGACGTGCACCCCAGCTGCAAGGTGACCGCCATGAAGTGC
TTTCTGCTGGAACTGCAGGTGATCAGCCTGGAAAGCGGCGACGCC
AGCATCCACGACACCGTGGAGAACCTGATCATCCTGGCCAACAAC
AGCCTGAGCAGCAACGGCAACGTGACCGAGAGCGGCTGCAAAGAG
TGCGAGGAACTGGAAGAGAAGAACATCAAAGAGTTTCTGCAGAGC
TTCGTGCACATCGTGCAGATGTTCATCAACACCAGCTGA

A corresponding amino acid sequence for CD5-IgG1-CD28TMD-41BB-CD3zeta-2A-IL15 is as follows:

(SEQ ID NO: 38)
MLEMEFGLSWLFLVAILKGVQCSRDIKMTQSPSSMYASLGERVTI
TCKASQDINSYLSWFQQKPGKSPKTLIYRANRLVDGVPSRFSGSG
SGQDYSLTISSLDYEDMGIYYCQQYDESPWTFGGGTKLEIKGSTS
GSGKPGSGEGSTKGQIQLVQSGPELKKPGETVKISCKASGYTFTN
YGMNWVKQAPGKGLRWMGWINTHTGEPTYADDFKGRFAFSLETSA
STAYLQINNLKNEDTATYFCTRRGYDWYFDVWGAGTTVTVSSRTV
TVSSQDPAEPKSPDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGKKDPKFWFWVLVVVGGVLACYSLLVTVAFII
FWVKRGRKKLLYIFKQPFMRPVQTTQEEDGCSCRFPEEEEGGCEL
TRVKFSRSADAPAYQQGQNQLYNELNLGRREEYDVLDKRRGRDPE
MGGKPRRKNPQEGLYNELQKDKMAEAYSEIGMKGERRRGKGHDGL
YQGLSTATKDTYDALHMQALPPRGPQCTNYALLKLAGDVESNPGP
MRISKPHLRSISIQCYLCLLLNSHFLTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS

In one example, an anti-CD5 CAR comprising an IgG1 hinge, CD28 transmembrane domain and DAP10 intracytoplasmic domain, and CD3zeta is utilized. In a vector, the CAR may be expressed with IL-15, such as may be separated from the CAR by a 2A sequence. In a specific example, such a CAR and IL-15 construct may have the following nucleotide sequence:

### CD5-IgG1-CD28TMD-DAP10 ICD.CD3zeta-2A-IL15

(SEQ ID NO: 39)
ATGCTCGAGATGGAGTTCGGCTTGAGTTGGTTGTTCCTTGTGGCG
ATACTCAAAGGCGTTCAATGTAGCCGAGACATAAAAATGACCCAG
TCTCCGTCATCTATGTATGCAAGCCTCGGCGAGCGAGTGACCATC
ACGTGCAAGGCGAGTCAAGATATAAACAGCTACTTGTCATGGTTC
CAACAAAAACCAGGGAAATCACCTAAGACCCTGATCTATAGAGCC
AATCGCCTGGTTGACGGTGTCCCCTCCCGCTTTAGCGGCTCCGGA
AGCGGTCAAGATTACTCTCTCACAATTTCTTCCTTGGATTATGAA
GACATGGGGATCTACTATTGTCAACAGTATGACGAATCCCCGTGG
ACTTTCGGTGGCGGTACCAAATTGGAAATAAAGGGCTCTACAAGC
GGCTCAGGAAAACCTGGATCAGGCGAAGGGTCTACGAAGGGCCAG
ATACAACTCGTTCAAAGTGGGCCAGAACTCAAAAAACCGGGAGAA
ACAGTGAAAATTTCTTGTAAGGCATCAGGATACACATTCACAAAC
TACGGGATGAATTGGGTCAAACAAGCACCCGGAAAGGGGCTGCGC
TGGATGGGGTGGATCAACACACATACTGGGGAACCTACTTACGCA
GACGATTTCAAGGGCAGATTCGCCTTTTCTTTGGAGACCTCCGCC
TCTACTGCATACTTGCAGATAAACAACCTGAAGAATGAAGATACC
GCCACCTACTTCTGTACGCGCAGGGGCTACGATTGGTATTTTGAT
GTATGGGGGGCAGGCACCACTGTTACTGTGTCAAGCCGTACGGTC
ACTGTCTCTTCACAGGATCCCGCCGAGCCCAAATCTCCTGACAAA
ACTCACACATGCCCACCGTGCCCAGCACCTGAACTCCTGGGGGGA
CCGTCAGTCTTCCTCTTCCCCCCAAAACCCAAGGACACCCTCATG
ATCTCCCGGACCCCTGAGGTCACATGCGTGGTGGTGGACGTGAGC
CACGAAGACCCTGAGGTCAAGTTCAACTGGTACGTGGACGGCGTG
GAGGTGCATAATGCCAAGACAAAGCCGCGGGAGGAGCAGTACAAC
AGCACGTACCGTGTGGTCAGCGTCCTCACCGTCCTGCACCAGGAC
TGGCTGAATGGCAAGGAGTACAAGTGCAAGGTCTCCAACAAAGCC
CTCCCAGCCCCCATCGAGAAAACCATCTCCAAAGCCAAAGGGCAG
CCCCGAGAACCACAGGTGTACACCCTGCCCCCATCCCGGGATGAG
CTGACCAAGAACCAGGTCAGCCTGACCTGCCTGGTCAAAGGCTTC
TATCCCAGCGACATCGCCGTGGAGTGGGAGAGCAATGGGCAACCG
GAGAACAACTACAAGACCACGCCTCCCGTGCTGGACTCCGACGGC
TCCTTCTTCCTCTACAGCAAGCTCACCGTGGACAAGAGCAGGTGG
CAGCAGGGGAACGTCTTCTCATGCTCCGTGATGCATGAGGCTCTG
CACAACCACTACACGCAGAAGAGCCTCTCCCTGTCTCCGGGTAAA
AAAGATCCCAAATTTTGGTTTTGGGTGCTGGTGGTGGTTGGTGGA
GTCCTGGCTTGCTATAGCTTGCTAGTAACAGTGGCCTTTATTATT
TTCTGGGTGCTTTGCGCACGCCCACGCCGCAGCCCCGCCCAAGAA
GATGGCAAAGTCTACATCAACATGCCAGGCAGGGGCACGCGTgtG
AAGTTCAGCAGGAGCGCAGACGCCCCCGCGTACCAGCAGGGCCAG
AACCAGCTCTATAACGAGCTCAATCTAGGACGAAGAGAGGAGTAC
GATGTTTTGGACAAAAGACGTGGCCGGGACCCTGAGATGGGGGGA
AAGCCGAGAAGGAAGAACCCTCAGGAAGGCCTGTACAATGAACTG
CAGAAAGATAAGATGGCGGAGGCCTACAGTGAGATTGGGATGAAA
GGCGAGCGCCGGAGGGGCAAGGGGCACGATGGCCTTTACCAGGGT
CTCAGTACAGCCACCAAGGACACCTACGACGCCCTTCACATGCAG
GCCCTGCCCCCTCGCGGACCGCAGTGTACTAATTATGCTCTCTTG
AAATTGGCTGGAGATGTTGAGAGCAATCCCGGGCCCATGCGCATT
AGCAAGCCCCACCTGCGGAGCATCAGCATCCAGTGCTACCTGTGC
CTGCTGCTGAACAGCCACTTCCTGACCGAGGCCGGCATCCACGTG
TTCATCCTGGGCTGCTTCAGCGCCGGACTGCCCAAGACCGAGGCC
AACTGGGTGAACGTGATCAGCGACCTGAAGAAGATCGAGGACCTG
ATCCAGAGCATGCACATCGACGCCACCCTGTACACCGAGAGCGAC
GTGCACCCCAGCTGCAAGGTGACCGCCATGAAGTGCTTTCTGCTG
GAACTGCAGGTGATCAGCCTGGAAAGCGGCGACGCCAGCATCCAC
GACACCGTGGAGAACCTGATCATCCTGGCCAACAACAGCCTGAGC
AGCAACGGCAACGTGACCGAGAGCGGCTGCAAAGAGTGCGAGGAA
CTGGAAGAGAAGAACATCAAAGAGTTTCTGCAGAGCTTCGTGCAC
ATCGTGCAGATGTTCATCAACACCAGCTGA

A corresponding amino acid sequence for CD5-IgG1-CD28TMD-DAP10 ICD.CD3zeta-2A-IL15 is as follows:

(SEQ ID NO: 40)
MLEMEFGLSWLFLVAILKGVQCSRDIKMTQSPSSMYASLGERVTITCKASQDINS
YLSWFQQKPGKSPKTLIYRANRLVDGVPSRFSGSGSGQDYSLTISSLDYEDMGIYYCQQ
YDESPWTFGGGTKLEIKGSTSGSGKPGSGEGSTKGQIQLVQSGPELKKPGETVKISCKAS
GYTFTNYGMNWVKQAPGKGLRWMGWINTHTGEPTYADDFKGRFAFSLETSASTAYLQ
INNLKNEDTATYFCTRRGYDWYFDVWGAGTTVTVSSRTVTVSSQDPAEPKSPDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKKDPKFWFWVLVV
VGGVLACYSLLVTVAFIIFWVLCARPRRSPAQEDGKVYINMPGRGTRVKFSRSADAPAY
QQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPRGPQCTNYALLKLAG
DVESNPGPMRISKPHLRSISIQCYLCLLLNSHFLTEAGIHVFILGCFSAGLPKTEANWVNVI
SDLKKIEDLIQSMHIDATLYTESDVHPSCKVTAMKCFLLELQVISLESGDASIHDTVENLII
LANNSLSSNGNVTESGCKECEELEEKNIKEFLQSFVHIVQMFINTS

In one example, an anti-CD5 CAR comprising an IgG1 hinge, DAP12 transmembrane domain and DAP12 intracytoplasmic domain, and CD3zeta is utilized. In a vector, the CAR may be expressed with IL-15, such as may be separated from the CAR by a 2A sequence. In a specific example, such a CAR and IL-15 construct may have the following nucleotide sequence:

### CD5-IgG1-DAP12TMD-DAP12 ICD.CD3zeta-2A-IL15

(SEQ ID NO: 41)
ATGCTCGAGATGGAGTTCGGCTTGAGTTGGTTGTTCCTTGTGGCGATACTCAAAGGC
GTTCAATGTAGCCGAGACATAAAAATGACCCAGTCTCCGTCATCTATGTATGCAAGC
CTCGGCGAGCGAGTGACCATCACGTGCAAGGCGAGTCAAGATATAAACAGCTACTT
GTCATGGTTCCAACAAAAACCAGGGAAATCACCTAAGACCCTGATCTATAGAGCCA
ATCGCCTGGTTGACGGTGTCCCCTCCCGCTTTAGCGGCTCCGGAAGCGGTCAAGATT
ACTCTCTCACAATTTCTTCCTTGGATTATGAAGACATGGGGATCTACTATTGTCAACA
GTATGACGAATCCCCGTGGACTTTCGGTGGCGGTACCAAATTGGAAATAAAGGGCT
CTACAAGCGGCTCAGGAAAACCTGGATCAGGCGAAGGGTCTACGAAGGGCCAGATA
CAACTCGTTCAAAGTGGGCCAGAACTCAAAAAACCGGGAGAAACAGTGAAAATTTC
TTGTAAGGCATCAGGATACACATTCACAAACTACGGGATGAATTGGGTCAAACAAG
CACCCGGAAAGGGGCTGCGCTGGATGGGGTGGATCAACACACATACTGGGGAACCT
ACTTACGCAGACGATTTCAAGGGCAGATTCGCCTTTTCTTTGGAGACCTCCGCCTCT
ACTGCATACTTGCAGATAAACAACCTGAAGAATGAAGATACCGCCACCTACTTCTGT
ACGCGCAGGGGCTACGATTGGTATTTTGATGTATGGGGGGCAGGCACCACTGTTACT
GTGTCAAGCCGTACGGTCACTGTCTCTTCACAGGATCCCGCCGAGCCCAAATCTCCT
GACAAAACTCACACATGCCCACCGTGCCCAGCACCTGAACTCCTGGGGGGACCGTC
AGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCA
GTACAACAGCACGTACCGTGTGGTCAGCGTCCTCACCGTCCTGCACCAGGACTGGCT
GAATGGCAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAA
AGGCTTCTATCCCAGCGACATCGCCGTGGAGTGGGAGAGCAATGGGCAACCGGAGA
ACAACTACAAGACCACGCCTCCCGTGCTGGACTCCGACGGCTCCTTCTTCCTCTACA
GCAAGCTCACCGTGGACAAGAGCAGGTGGCAGCAGGGGAACGTCTTCTCATGCTCC
GTGATGCATGAGGCTCTGCACAACCACTACACGCAGAAGAGCCTCTCCCTGTCTCCG
GGTAAAAAAGATCCCAAATTTTGGGGCGTGCTGGCAGGGATCGTGATGGGAGACCT
GGTGCTGACAGTGCTCATTGCCCTGGCCGTGTACTTCCTGGGCCGGCTGGTCCCTCG
GGGGCGAGGGGCTGCGGAGGCAGCGACCCGGAAACAGCGTATCACTGAGACCGAG
TCGCCTTATCAGGAGCTCCAGGGTCAGAGGTCGGATGTCTACAGCGACCTCAACAC
ACAGAGGCCGTATTACAAAACGCGTgtGAAGTTCAGCAGGAGCGCAGACGCCCCCGC
GTACCAGCAGGGCCAGAACCAGCTCTATAACGAGCTCAATCTAGGACGAAGAGAGG
AGTACGATGTTTTGGACAAAAGACGTGGCCGGGACCCTGAGATGGGGGGAAAGCCG
AGAAGGAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAGGGGCA
CGATGGCCTTTACCAGGGTCTCAGTACAGCCACCAAGGACACCTACGACGCCCTTCA
CATGCAGGCCCTGCCCCCTCGCGGACCGCAGTGTACTAATTATGCTCTCTTGAAATT
GGCTGGAGATGTTGAGAGCAATCCCGGGCCCATGCGCATTAGCAAGCCCCACCTGC
GGAGCATCAGCATCCAGTGCTACCTGTGCCTGCTGCTGAACAGCCACTTCCTGACCG
AGGCCGGCATCCACGTGTTCATCCTGGGCTGCTTCAGCGCCGGACTGCCCAAGACCG
AGGCCAACTGGGTGAACGTGATCAGCGACCTGAAGAAGATCGAGGACCTGATCCAG
AGCATGCACATCGACGCCACCCTGTACACCGAGAGCGACGTGCACCCCAGCTGCAA
GGTGACCGCCATGAAGTGCTTTCTGCTGGAACTGCAGGTGATCAGCCTGGAAAGCG
GCGACGCCAGCATCCACGACACCGTGGAGAACCTGATCATCCTGGCCAACAACAGC
CTGAGCAGCAACGGCAACGTGACCGAGAGCGGCTGCAAAGAGTGCGAGGAACTGG
AAGAGAAGAACATCAAAGAGTTTCTGCAGAGCTTCGTGCACATCGTGCAGATGTTC
ATCAACACCAGCTGA

A corresponding amino acid sequence for CD5-IgG1-DAP12TMD-DAP12 ICD.CD3zeta-2A-IL15 is as follows:

(SEQ ID NO: 42)
MLEMEFGLSWLFLVAILKGVQCSRDIKMTQSPSSMYASLGERVTITCKASQDINS
YLSWFQQKPGKSPKTLIYRANRLVDGVPSRFSGSGSGQDYSLTISSLDYEDMGIYYCQQ
YDESPWTFGGGTKLEIKGSTSGSGKPGSGEGSTKGQIQLVQSGPELKKPGETVKISCKAS
GYTFTNYGMNWVKQAPGKGLRWMGWINTHTGEPTYADDFKGRFAFSLETSASTAYLQ
INNLKNEDTATYFCTRRGYDWYFDVWGAGTTVTVSSRTVTVSSQDPAEPKSPDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKKDPKFWGVLAGIV
MGDLVLTVLIALAVYFLGRLVPRGRGAAEAATRKQRITETESPYQELQGQRSDVYSDLN
TQRPYYKTRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALH
MQALPPRGPQCTNYALLKLAGDVESNPGPMRISKPHLRSISIQCYLCLLLNSHFLTEAGIH
VFILGCFSAGLPKTEANWVNVISDLKKIEDLIQSMHIDATLYTESDVHPSCKVTAMKCFL
LELQVISLESGDASIHDTVENLIILANNSLSSNGNVTESGCKECEELEEKNIKEFLQSFVHI
VQMFINTS

In one example, an anti-CD5 CAR comprising an IgG1 hinge, CD28 transmembrane domain and DAP12 intracytoplasmic domain, and CD3zeta is utilized. In a vector, the CAR may be expressed with IL-15, such as may be separated from the CAR by a 2A sequence. In a specific example, such a CAR and IL-15 construct may have the following nucleotide sequence:

### CD5-IgG1-CD28TMD-DAP12 ICD.CD3zeta-2A-IL15

(SEQ ID NO: 43)
ATGCTCGAGatggagttcggcttgagttggttgttccttgtggcgatactcaaaggcgttcaatgtagccgagacataaaa
atgacccagtctccgtcatctatgtatgcaagcctcggcgagcgagtgaccatcacgtgcaaggcgagtcaagatataaacagctacttgtc
atggttccaacaaaaaccagggaaatcacctaagaccctgatctatagagccaatcgcctggttgacggtgtcccctcccgctttagcggct
ccggaagcggtcaagattactctctcacaatttcttccttggattatgaagacatggggatctactattgtcaacagtatgacgaatccccgtgg
actttcggtggcggtaccaaattggaaataaagggctctacaagcggctcaggaaaacctggatcaggcgaagggtctacgaagggcca
gatacaactcgttcaaagtgggccagaactcaaaaaaccgggagaaacagtgaaaatttcttgtaaggcatcaggatacacattcacaaact
acgggatgaattgggtcaaacaagcacccggaaaggggctgcgctggatggggggatcaacacacatactggggaacctacttacgca
gacgatttcaagggcagattcgccttttctttggagacctccgcctctactgcatacttgcagataaacaacctgaagaatgaagataccgcca
cctacttctgtacgcgcaggggctacgattggtattttgatgtatggggggcaggcaccactgttactgtgtcaagcCGTACGgTCA
CTGTCTCTTCACAGGATCCCGCCGAGCCCAAATCTCCTGACAAAACTCACACATGCC
CACCGTGCCCAGCACCTGAACTCCTGGGGGGACCGTCAGTCTTCCTCTTCCCCCCAA
AACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGA
GGTGCATAATGCCAAGACAAAGCCGCGGGAGGAGCAGTACAACAGCACGTACCGT
GTGGTCAGCGTCCTCACCGTCCTGCACCAGGACTGGCTGAATGGCAAGGAGTACAA
GTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGATGAG
CTGACCAAGAACCAGGTCAGCCTGACCTGCCTGGTCAAAGGCTTCTATCCCAGCGAC
ATCGCCGTGGAGTGGGAGAGCAATGGGCAACCGGAGAACAACTACAAGACCACGC
CTCCCGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAAAAAGATCCCAA
ATTTTGGTTTTGGGTGCTGGTGGTGGTTGGTGGAGTCCTGGCTTGCTATAGCTTGCTA
GTAACAGTGGCCTTTATTATTTTCTGGGTGTACTTCCTGGGCCGGCTGGTCCCTCGGG
GGCGAGGGGCTGCGGAGGCAGCGACCCGGAAACAGCGTATCACTGAGACCGAGTC
GCCTTATCAGGAGCTCCAGGGTCAGAGGTCGGATGTCTACAGCGACCTCAACACAC
AGAGGCCGTATTACAAAACGCGTgtGAAGTTCAGCAGGAGCGCAGACGCCCCCGCGT
ACCAGCAGGGCCAGAACCAGCTCTATAACGAGCTCAATCTAGGACGAAGAGAGGA
GTACGATGTTTTGGACAAAAGACGTGGCCGGGACCCTGAGATGGGGGGAAAGCCGA
GAAGGAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTCAC
ATGCAGGCCCTGCCCCCTCGCGGACCGCAGTGTACTAATTATGCTCTCTTGAAATTG
GCTGGAGATGTTGAGAGCAATCCCGGGCCCATGCGCATTAGCAAGCCCCACCTGCG
GAGCATCAGCATCCAGTGCTACCTGTGCCTGCTGCTGAACAGCCACTTCCTGACCGA
GGCCGGCATCCACGTGTTCATCCTGGGCTGCTTCAGCGCCGGACTGCCCAAGACCGA
GGCCAACTGGGTGAACGTGATCAGCGACCTGAAGAAGATCGAGGACCTGATCCAGA
GCATGCACATCGACGCCACCCTGTACACCGAGAGCGACGTGCACCCCAGCTGCAAG
GTGACCGCCATGAAGTGCTTTCTGCTGGAACTGCAGGTGATCAGCCTGGAAAGCGG
CGACGCCAGCATCCACGACACCGTGGAGAACCTGATCATCCTGGCCAACAACAGCC
TGAGCAGCAACGGCAACGTGACCGAGAGCGGCTGCAAAGAGTGCGAGGAACTGGA
AGAGAAGAACATCAAAGAGTTTCTGCAGAGCTTCGTGCACATCGTGCAGATGTTCAT
CAACACCAGCTGA

A corresponding amino acid sequence for CD5-IgG1-CD28TMD-DAP12 ICD.CD3zeta-2A-IL15 is as follows:

(SEQ ID NO: 44)
MLEMEFGLSWLFLVAILKGVQCSRDIKMTQSPSSMYASLGERVTITCKASQDINS
YLSWFQQKPGKSPKTLIYRANRLVDGVPSRFSGSGSGQDYSLTISSLDYEDMGIYYCQQ
YDESPWTFGGGTKLEIKGSTSGSGKPGSGEGSTKGQIQLVQSGPELKKPGETVKISCKAS
GYTFTNYGMNWVKQAPGKGLRWMGWINTHTGEPTYADDFKGRFAFSLETSASTAYLQ
INNLKNEDTATYFCTRRGYDWYFDVWGAGTTVTVSSRTVTVSSQDPAEPKSPDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKKDPKFWFWVLVV
VGGVLACYSLLVTVAFIIFWVYFLGRLVPRGRGAAEAATRKQRITETESPYQELQGQRS
DVYSDLNTQRPYYKTRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATKD
TYDALHMQALPPRGPQCTNYALLKLAGDVESNPGPMRISKPHLRSISIQCYLCLLLNSHF
LTEAGIHVFILGCFSAGLPKTEANWVNVISDLKKIEDLIQSMHIDATLYTESDVHPSCKVT
AMKCFLLELQVISLESGDASIHDTVENLIILANNSLSSNGNVTESGCKECEELEEKNIKEFL
QSFVHIVQMFINTS

In one example, an anti-CD5 CAR comprising an IgG1 hinge, NKG2D transmembrane domain and NKG2D intracytoplasmic domain, and CD3zeta is utilized. In a vector, the CAR may be expressed with IL-15, such as may be separated from the CAR by a 2A sequence. In a specific example, such a CAR and IL-15 construct may have the following nucleotide sequence:

### CD5-IgG1-NKG2DTMD-NKG2D ICD.CD3zeta-2A-IL15

(SEQ ID NO: 45)
ATGCTCGAGATGGAGTTCGGCTTGAGTTGGTTGTTCCTTGTGGCGATACTCAA
AGGCGTTCAATGTAGCCGAGACATAAAAATGACCCAGTCTCCGTCATCTATGTATGC
AAGCCTCGGCGAGCGAGTGACCATCACGTGCAAGGCGAGTCAAGATATAAACAGCT
ACTTGTCATGGTTCCAACAAAAACCAGGGAAATCACCTAAGACCCTGATCTATAGA
GCCAATCGCCTGGTTGACGGTGTCCCCTCCCGCTTTAGCGGCTCCGGAAGCGGTCAA
GATTACTCTCTCACAATTTCTTCCTTGGATTATGAAGACATGGGGATCTACTATTGTC
AACAGTATGACGAATCCCCGTGGACTTTCGGTGGCGGTACCAAATTGGAAATAAAG
GGCTCTACAAGCGGCTCAGGAAAACCTGGATCAGGCGAAGGGTCTACGAAGGGCCA
GATACAACTCGTTCAAAGTGGGCCAGAACTCAAAAAACCGGGAGAAACAGTGAAA
ATTTCTTGTAAGGCATCAGGATACACATTCACAAACTACGGGATGAATTGGGTCAAA
CAAGCACCCGGAAAGGGGCTGCGCTGGATGGGGTGGATCAACACACATACTGGGGA
ACCTACTTACGCAGACGATTTCAAGGGCAGATTCGCCTTTTCTTTGGAGACCTCCGC
CTCTACTGCATACTTGCAGATAAACAACCTGAAGAATGAAGATACCGCCACCTACTT
CTGTACGCGCAGGGGCTACGATTGGTATTTTGATGTATGGGGGGCAGGCACCACTGT
TACTGTGTCAAGCCGTACGGTCACTGTCTCTTCACAGGATCCCGCCGAGCCCAAATC
TCCTGACAAAACTCACACATGCCCACCGTGCCCAGCACCTGAACTCCTGGGGGGAC
CGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGG
AGCAGTACAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACAC
CCTGCCCCCATCCCGGGATGAGCTGACCAAGAACCAGGTCAGCCTGACCTGCCTGGT
CAAAGGCTTCTATCCCAGCGACATCGCCGTGGAGTGGGAGAGCAATGGGCAACCGG
AGAACAACTACAAGACCACGCCTCCCGTGCTGGACTCCGACGGCTCCTTCTTCCTCT
ACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCT
CCGGGTAAAAAAGATCCCAAATTTTGGGCGGTGATGATTATTTTTCGCATTGGCATG
GCGGTGGCGATTTTTTGCTGCTTTTTTTTTCCGAGCGCGAACGAACGCTGCAAAAGC
AAAGTGGTGCCGTGCCGCCAGAAACAGTGGCGCACCAGCTTTGATAGCAAAAAACT
GGATCTGAACTATAACCATTTTGAAAGCATGGAATGGAGCCATCGCAGCCGCCGCG
GCCGCATTTGGGGCATGACGCGTgtGAAGTTCAGCAGGAGCGCAGACGCCCCCGCGT
ACCAGCAGGGCCAGAACCAGCTCTATAACGAGCTCAATCTAGGACGAAGAGAGGA
GTACGATGTTTTGGACAAAAGACGTGGCCGGGACCCTGAGATGGGGGGAAAGCCGA
GAAGGAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTCAC
ATGCAGGCCCTGCCCCCTCGCGGACCGCAGTGTACTAATTATGCTCTCTTGAAATTG
GCTGGAGATGTTGAGAGCAATCCCGGGCCCATGCGCATTAGCAAGCCCCACCTGCG
GAGCATCAGCATCCAGTGCTACCTGTGCCTGCTGCTGAACAGCCACTTCCTGACCGA
GGCCGGCATCCACGTGTTCATCCTGGGCTGCTTCAGCGCCGGACTGCCCAAGACCGA
GGCCAACTGGGTGAACGTGATCAGCGACCTGAAGAAGATCGAGGACCTGATCCAGA
GCATGCACATCGACGCCACCCTGTACACCGAGAGCGACGTGCACCCCAGCTGCAAG
GTGACCGCCATGAAGTGCTTTCTGCTGGAACTGCAGGTGATCAGCCTGGAAAGCGG
CGACGCCAGCATCCACGACACCGTGGAGAACCTGATCATCCTGGCCAACAACAGCC
TGAGCAGCAACGGCAACGTGACCGAGAGCGGCTGCAAAGAGTGCGAGGAACTGGA
AGAGAAGAACATCAAAGAGTTTCTGCAGAGCTTCGTGCACATCGTGCAGATGTTCAT
CAACACCAGCTGA

A corresponding amino acid sequence for CD5-IgG1-NKG2DTMD-NKG2D ICD.CD3zeta-2A-IL15 is as follows:

(SEQ ID NO: 46)
MLEMEFGLSWLFLVAILKGVQCSRDIKMTQSPSSMYASLGERVTITCKASQDINS
YLSWFQQKPGKSPKTLIYRANRLVDGVPSRFSGSGSGQDYSLTISSLDYEDMGIYYCQQ
YDESPWTFGGGTKLEIKGSTSGSGKPGSGEGSTKGQIQLVQSGPELKKPGETVKISCKAS
GYTFTNYGMNWVKQAPGKGLRWMGWINTHTGEPTYADDFKGRFAFSLETSASTAYLQ
INNLKNEDTATYFCTRRGYDWYFDVWGAGTTVTVSSRTVTVSSQDPAEPKSPDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKKDPKFWAVMIIFRI
GMAVAIFCCFFFPSANERCKSKVVPCRQKQWRTSFDSKKLDLNYNHFESMEWSHRSRR
GRIWGMTRVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDTYDALHM
QALPPRGPQCTNYALLKLAGDVESNPGPMRISKPHLRSISIQCYLCLLLNSHFLTEAGIHV
FILGCFSAGLPKTEANWVNVISDLKKIEDLIQSMHIDATLYTESDVHPSCKVTAMKCFLL
ELQVISLESGDASIHDTVENLIILANNSLSSNGNVTESGCKECEELEEKNIKEFLQSFVHIV
QMFINTS

In one example, an anti-CD5 CAR comprising an IgG1 hinge, CD28 transmembrane domain and NKG2D intracytoplasmic domain, and CD3zeta is utilized. In a vector, the CAR may be expressed with IL-15, such as may be separated from the CAR by a 2A sequence. In a specific example, such a CAR and IL-15 construct may have the following nucleotide sequence:

### CD5-IgG1-CD28TMD-NKG2D ICD.CD3zeta-2A-IL15

(SEQ ID NO: 47)
ATGCTCGAGATGGAGTTCGGCTTGAGTTGGTTGTTCCTTGTGGCGATACTCAA
AGGCGTTCAATGTAGCCGAGACATAAAAATGACCCAGTCTCCGTCATCTATGTATGC
AAGCCTCGGCGAGCGAGTGACCATCACGTGCAAGGCGAGTCAAGATATAAACAGCT
ACTTGTCATGGTTCCAACAAAAACCAGGGAAATCACCTAAGACCCTGATCTATAGA
GCCAATCGCCTGGTTGACGGTGTCCCCTCCCGCTTTAGCGGCTCCGGAAGCGGTCAA
GATTACTCTCTCACAATTTCTTCCTTGGATTATGAAGACATGGGGATCTACTATTGTC
AACAGTATGACGAATCCCCGTGGACTTTCGGTGGCGGTACCAAATTGGAAATAAAG
GGCTCTACAAGCGGCTCAGGAAAACCTGGATCAGGCGAAGGGTCTACGAAGGGCCA
GATACAACTCGTTCAAAGTGGGCCAGAACTCAAAAAACCGGGAGAAACAGTGAAA
ATTTCTTGTAAGGCATCAGGATACACATTCACAAACTACGGGATGAATTGGGTCAAA
CAAGCACCCGGAAAGGGGCTGCGCTGGATGGGGTGGATCAACACACATACTGGGGA
ACCTACTTACGCAGACGATTTCAAGGGCAGATTCGCCTTTTCTTTGGAGACCTCCGC
CTCTACTGCATACTTGCAGATAAACAACCTGAAGAATGAAGATACCGCCACCTACTT
CTGTACGCGCAGGGGCTACGATTGGTATTTTGATGTATGGGGGGCAGGCACCACTGT
TACTGTGTCAAGCCGTACGGTCACTGTCTCTTCACAGGATCCCGCCGAGCCCAAATC
TCCTGACAAAACTCACACATGCCCACCGTGCCCAGCACCTGAACTCCTGGGGGGAC
CGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGG
AGCAGTACAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACAC
CCTGCCCCCATCCCGGGATGAGCTGACCAAGAACCAGGTCAGCCTGACCTGCCTGGT
CAAAGGCTTCTATCCCAGCGACATCGCCGTGGAGTGGGAGAGCAATGGGCAACCGG
AGAACAACTACAAGACCACGCCTCCCGTGCTGGACTCCGACGGCTCCTTCTTCCTCT
ACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCT
CCGGGTAAAAAAGATCCCAAATTTTGGTTTTGGGTGCTGGTGGTGGTTGGTGGAGTC
CTGGCTTGCTATAGCTTGCTAGTAACAGTGGCCTTTATTATTTTCTGGGTGAGCGCGA
ACGAACGCTGCAAAAGCAAAGTGGTGCCGTGCCGCCAGAAACAGTGGCGCACCAGC
TTTGATAGCAAAAAACTGGATCTGAACTATAACCATTTTGAAAGCATGGAATGGAG
CCATCGCAGCCGCCGCGGCCGCATTTGGGGCATGACGCGTgtGAAGTTCAGCAGGAG
CGCAGACGCCCCCGCGTACCAGCAGGGCCAGAACCAGCTCTATAACGAGCTCAATC
TAGGACGAAGAGAGGAGTACGATGTTTTGGACAAAAGACGTGGCCGGGACCCTGAG
ATGGGGGGAAAGCCGAGAAGGAAGAACCCTCAGGAAGGCCTGTACAATGAACTGC
AGAAAGATAAGATGGCGGAGGCCTACAGTGAGATTGGGATGAAAGGCGAGCGCCG
GAGGGGCAAGGGGCACGATGGCCTTTACCAGGGTCTCAGTACAGCCACCAAGGACA
CCTACGACGCCCTTCACATGCAGGCCCTGCCCCCTCGCGGACCGCAGTGTACTAATT
ATGCTCTCTTGAAATTGGCTGGAGATGTTGAGAGCAATCCCGGGCCCATGCGCATTA
GCAAGCCCCACCTGCGGAGCATCAGCATCCAGTGCTACCTGTGCCTGCTGCTGAACA
GCCACTTCCTGACCGAGGCCGGCATCCACGTGTTCATCCTGGGCTGCTTCAGCGCCG
GACTGCCCAAGACCGAGGCCAACTGGGTGAACGTGATCAGCGACCTGAAGAAGATC
GAGGACCTGATCCAGAGCATGCACATCGACGCCACCCTGTACACCGAGAGCGACGT
GCACCCCAGCTGCAAGGTGACCGCCATGAAGTGCTTTCTGCTGGAACTGCAGGTGAT
CAGCCTGGAAAGCGGCGACGCCAGCATCCACGACACCGTGGAGAACCTGATCATCC
TGGCCAACAACAGCCTGAGCAGCAACGGCAACGTGACCGAGAGCGGCTGCAAAGA
GTGCGAGGAACTGGAAGAGAAGAACATCAAAGAGTTTCTGCAGAGCTTCGTGCACA
TCGTGCAGATGTTCATCAACACCAGCTGA

A corresponding amino acid sequence for CD5-IgG1-CD28TMD-NKG2D ICD.CD3zeta-2A-IL15 is as follows:

(SEQ ID NO: 48)
MLEMEFGLSWLFLVAILKGVQCSRDIKMTQSPSSMYASLGERVTITCKASQDINS
YLSWFQQKPGKSPKTLIYRANRLVDGVPSRFSGSGSGQDYSLTISSLDYEDMGIYYCQQ
YDESPWTFGGGTKLEIKGSTSGSGKPGSGEGSTKGQIQLVQSGPELKKPGETVKISCKAS
GYTFTNYGMNWVKQAPGKGLRWMGWINTHTGEPTYADDFKGRFAFSLETSASTAYLQ
INNLKNEDTATYFCTRRGYDWYFDVWGAGTTVTVSSRTVTVSSQDPAEPKSPDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKKDPKFWFWVLVV
VGGVLACYSLLVTVAFIIFWVSANERCKSKVVPCRQKQWRTSFDSKKLDLNYNHFESM
EWSHRSRRGRIWGMTRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATKD
TYDALHMQALPPRGPQCTNYALLKLAGDVESNPGPMRISKPHLRSISIQCYLCLLLNSHF
LTEAGIHVFILGCFSAGLPKTEANWVNVISDLKKIEDLIQSMHIDATLYTESDVHPSCKVT
AMKCFLLELQVISLESGDASIHDTVENLIILANNSLSSNGNVTESGCKECEELEEKNIKEFL
QSFVHIVQMFINTS

In one example, an anti-CD5 CAR comprising an CD28 hinge, CD28 transmembrane domain and DAP10 intracytoplasmic domain, and CD3zeta is utilized. In a vector, the CAR may or may not be expressed with IL-15, such as may be separated from the CAR by a 2A sequence, as one example. In a specific example, such a CAR and IL-15 construct may have the following nucleotide sequence:

iC9.CD5CARCD28hinge.tmdDAP103z.IL15

(SEQ ID NO: 52)
ATGCTCGAGGGAGTGCAGGTGGAAACCATCTCCCCAGGCGACGGGCGCACCT
TCCCCAAGCGCGGCCAGACCTGCGTGGTGCACTACACCGGGATGCTTGAAGATGGA
AAGAAAGTTGATTCCTCCCGGGACAGAAACAAGCCCTTTAAGTTTATGCTAGGCAA
GCAGGAGGTGATCCGAGGCTGGGAAGAAGGGGTTGCCCAGATGAGTGTGGGTCAGA
GAGCCAAACTGACTATATCTCCAGATTATGCCTATGGTGCCACTGGGCACCCAGGCA
TCATCCCACCACATGCCACTCTCGTCTTCGATGTGGAGCTTCTAAAACTGGAATCTG
GCGGTGGATCCGGAGTCGACGGATTTGGTGATGTCGGTGCTCTTGAGAGTTTGAGGG
GAAATGCAGATTTGGCTTACATCCTGAGCATGGAGCCCTGTGGCCACTGCCTCATTA
TCAACAATGTGAACTTCTGCCGTGAGTCCGGGCTCCGCACCCGCACTGGCTCCAACA
TCGACTGTGAGAAGTTGCGGCGTCGCTTCTCCTCGCTGCATTTCATGGTGGAGGTGA
AGGGCGACCTGACTGCCAAGAAAATGGTGCTGGCTTTGCTGGAGCTGGCGCAGCAG
GACCACGGTGCTCTGGACTGCTGCGTGGTGGTCATTCTCTCTCACGGCTGTCAGGCC
AGCCACCTGCAGTTCCCAGGGGCTGTCTACGGCACAGATGGATGCCCTGTGTCGGTC
GAGAAGATTGTGAACATCTTCAATGGGACCAGCTGCCCCAGCCTGGGAGGGAAGCC
CAAGCTCTTTTTCATCCAGGCCTGTGGTGGGGAGCAGAAAGATCATGGGTTTGAGGT
GGCCTCCACTTCCCCTGAAGACGAGTCCCCTGGCAGTAACCCCGAGCCAGATGCCAC
CCCGTTCCAGGAAGGTTTGAGGACCTTCGACCAGCTGGACGCCATATCTAGTTTGCC
CACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGG
GCTCACTCTGAAGACCTGCAGTCCCTCCTGCTTAGGGTCGCTAATGCTGTTTCGGTG
AAAGGGATTTATAAACAGATGCCTGGTTGCTTTAATTTCCTCCGGAAAAAACTTTTC
TTTAAAACATCAGCTTCGCGAGCCGAGGGCAGGGGAAGTCTTCTAACATGCGGGGA
CGTGGAGGAAAATCCCGGGCCCCTCGAGATGGAGTTCGGCTTGAGTTGGTTGTTCCT
TGTGGCGATACTCAAAGGCGTTCAATGTAGCCGAGACATAAAAATGACCCAGTCTC
CGTCATCTATGTATGCAAGCCTCGGCGAGCGAGTGACCATCACGTGCAAGGCGAGT
CAAGATATAAACAGCTACTTGTCATGGTTCCAACAAAAACCAGGGAAATCACCTAA
GACCCTGATCTATAGAGCCAATCGCCTGGTTGACGGTGTCCCCTCCCGCTTTAGCGG
CTCCGGAAGCGGTCAAGATTACTCTCTCACAATTTCTTCCTTGGATTATGAAGACAT
GGGGATCTACTATTGTCAACAGTATGACGAATCCCCGTGGACTTTCGGTGGCGGTAC
CAAATTGGAAATAAAGGGCTCTACAAGCGGCTCAGGAAAACCTGGATCAGGCGAAG
GGTCTACGAAGGGCCAGATACAACTCGTTCAAAGTGGGCCAGAACTCAAAAAACCG
GGAGAAACAGTGAAAATTTCTTGTAAGGCATCAGGATACACATTCACAAACTACGG
GATGAATTGGGTCAAACAAGCACCCGGAAAGGGGCTGCGCTGGATGGGGTGGATCA
ACACACATACTGGGGAACCTACTTACGCAGACGATTTCAAGGGCAGATTCGCCTTTT
CTTTGGAGACCTCCGCCTCTACTGCATACTTGCAGATAAACAACCTGAAGAATGAAG
ATACCGCCACCTACTTCTGTACGCGCAGGGGCTACGATTGGTATTTTGATGTATGGG
GGGCAGGCACCACTGTTACTGTGTCAAGCCGTACGATTGAAGTTATGTATCCTCCTC
CTTACCTAGACAATGAGAAGAGCAATGGAACCATTATCCATGTGAAAGGGAAACAC
CTTTGTCCAAGTCCCCTATTTCCCGGACCTTCTAAGCCCTTTTGGGTGCTGGTGGTGG
TTGGTGGAGTCCTGGCTTGCTATAGCTTGCTAGTAACAGTGGCCTTTATTATTTTCTG
GGTGCTTTGCGCACGCCCACGCCGCAGCCCCGCCCAAGAAGATGGCAAAGTCTACA
TCAACATGCCAGGCAGGGGCACGCGTGTGAAGTTCAGCAGGAGCGCAGACGCCCCC
GCGTACCAGCAGGGCCAGAACCAGCTCTATAACGAGCTCAATCTAGGACGAAGAGA
GGAGTACGATGTTTTGGACAAAAGACGTGGCCGGGACCCTGAGATGGGGGGAAAGC
CGAGAAGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCAAGGGG
CACGATGGCCTTTACCAGGGTCTCAGTACAGCCACCAAGGACACCTACGACGCCCTT
CACATGCAGGCCCTGCCCCCTCGCGGACCGCAGTGTACTAATTATGCTCTCTTGAAA
TTGGCTGGAGATGTTGAGAGCAATCCCGGGCCCATGCGCATTAGCAAGCCCCACCT
GCGGAGCATCAGCATCCAGTGCTACCTGTGCCTGCTGCTGAACAGCCACTTCCTGAC
CGAGGCCGGCATCCACGTGTTCATCCTGGGCTGCTTCAGCGCCGGACTGCCCAAGAC
CGAGGCCAACTGGGTGAACGTGATCAGCGACCTGAAGAAGATCGAGGACCTGATCC
AGAGCATGCACATCGACGCCACCCTGTACACCGAGAGCGACGTGCACCCCAGCTGC
AAGGTGACCGCCATGAAGTGCTTTCTGCTGGAACTGCAGGTGATCAGCCTGGAAAG
CGGCGACGCCAGCATCCACGACACCGTGGAGAACCTGATCATCCTGGCCAACAACA
GCCTGAGCAGCAACGGCAACGTGACCGAGAGCGGCTGCAAAGAGTGCGAGGAACT
GGAAGAGAAGAACATCAAAGAGTTTCTGCAGAGCTTCGTGCACATCGTGCAGATGT
TCATCAACACCAGCTGA

A corresponding amino acid sequence for iC9.CD5CARCD28hinge.tmdDAP103z.IL15 is as follows:

(SEQ ID NO: 53)
MLEGVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFDVELLKLESG
GGSGVDGFGDVGALESLRGNADLAYILSMEPCGHCLIINNVNFCRESGLRTRTGSNIDCE
KLRRRFSSLHFMVEVKGDLTAKKMVLALLELAQQDHGALDCCVVVILSHGCQASHLQF
PGAVYGTDGCPVSVEKIVNIFNGTSCPSLGGKPKLFFIQACGGEQKDHGFEVASTSPEDE
SPGSNPEPDATPFQEGLRTFDQLDAISSLPTPSDIFVSYSTFPGFVSWRDPKSGSWYVETL
DDIFEQWAHSEDLQSLLLRVANAVSVKGIYKQMPGCFNFLRKKLFFKTSASRAEGRGSL
LTCGDVEENPGPLEMEFGLSWLFLVAILKGVQCSRDIKMTQSPSSMYASLGERVTITCK
ASQDINSYLSWFQQKPGKSPKTLIYRANRLVDGVPSRFSGSGSGQDYSLTISSLDYEDMG
IYYCQQYDESPWTFGGGTKLEIKGSTSGSGKPGSGEGSTKGQIQLVQSGPELKKPGETVK
ISCKASGYTFTNYGMNWVKQAPGKGLRWMGWINTHTGEPTYADDFKGRFAFSLETSA
STAYLQINNLKNEDTATYFCTRRGYDWYFDVWGAGTTVTVSSRTIEVMYPPPYLDNEK
SNGTIIHVKGKHLCPSPLFPGPSKPFWVLVVVGGVLACYSLLVTVAFIIFWVLCARPRRSP
AQEDGKVYINMPGRGTRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATK
DTYDALHMQALPPRGPQCTNYALLKLAGDVESNPGPMRISKPHLRSISIQCYLCLLLNSH
FLTEAGIHVFILGCFSAGLPKTEANWVNVISDLKKIEDLIQSMHIDATLYTESDVHPSCKV
TAMKCFLLELQVISLESGDASIHDTVENLIILANNSLSSNGNVTESGCKECEELEEKNIKEF
LQSFVHIVQMFINTS

In one example, an anti-CD5 CAR comprising an CD28 hinge, CD28 transmembrane domain and CD28 intracytoplasmic domain, and CD3zeta is utilized. In a vector, the CAR may or may not be expressed with IL-15, such as may be separated from the CAR by a 2A sequence, as one example. In a specific example, such a CAR and IL-15 construct may have the following nucleotide sequence:

iC9.CD5CARCD28hinge.28tmd3z.IL15

(SEQ ID NO: 54)
ATGCTCGAGGGAGTGCAGGTGGAAACCATCTCCCCAGGCGACGGGCGCACCT
TCCCCAAGCGCGGCCAGACCTGCGTGGTGCACTACACCGGGATGCTTGAAGATGGA
AAGAAAGTTGATTCCTCCCGGGACAGAAACAAGCCCTTTAAGTTTATGCTAGGCAA
GCAGGAGGTGATCCGAGGCTGGGAAGAAGGGGTTGCCCAGATGAGTGTGGGTCAGA
GAGCCAAACTGACTATATCTCCAGATTATGCCTATGGTGCCACTGGGCACCCAGGCA
TCATCCCACCACATGCCACTCTCGTCTTCGATGTGGAGCTTCTAAAACTGGAATCTG
GCGGTGGATCCGGAGTCGACGGATTTGGTGATGTCGGTGCTCTTGAGAGTTTGAGGG
GAAATGCAGATTTGGCTTACATCCTGAGCATGGAGCCCTGTGGCCACTGCCTCATTA
TCAACAATGTGAACTTCTGCCGTGAGTCCGGGCTCCGCACCCGCACTGGCTCCAACA
TCGACTGTGAGAAGTTGCGGCGTCGCTTCTCCTCGCTGCATTTCATGGTGGAGGTGA
AGGGCGACCTGACTGCCAAGAAAATGGTGCTGGCTTTGCTGGAGCTGGCGCAGCAG
GACCACGGTGCTCTGGACTGCTGCGTGGTGGTCATTCTCTCTCACGGCTGTCAGGCC
AGCCACCTGCAGTTCCCAGGGGCTGTCTACGGCACAGATGGATGCCCTGTGTCGGTC
GAGAAGATTGTGAACATCTTCAATGGGACCAGCTGCCCCAGCCTGGGAGGGAAGCC
CAAGCTCTTTTTCATCCAGGCCTGTGGTGGGGAGCAGAAAGATCATGGGTTTGAGGT
GGCCTCCACTTCCCCTGAAGACGAGTCCCCTGGCAGTAACCCCGAGCCAGATGCCAC
CCCGTTCCAGGAAGGTTTGAGGACCTTCGACCAGCTGGACGCCATATCTAGTTTGCC
CACACCCAGTGACATCTTTGTGTCCTACTCTACTTTCCCAGGTTTTGTTTCCTGGAGG
GACCCCAAGAGTGGCTCCTGGTACGTTGAGACCCTGGACGACATCTTTGAGCAGTGG
GCTCACTCTGAAGACCTGCAGTCCCTCCTGCTTAGGGTCGCTAATGCTGTTTCGGTG
AAAGGGATTTATAAACAGATGCCTGGTTGCTTTAATTTCCTCCGGAAAAAACTTTTC
TTTAAAACATCAGCTTCGCGAGCCGAGGGCAGGGGAAGTCTTCTAACATGCGGGGA
CGTGGAGGAAAATCCCGGGCCCCTCGAGATGGAGTTCGGCTTGAGTTGGTTGTTCCT
TGTGGCGATACTCAAAGGCGTTCAATGTAGCCGAGACATAAAAATGACCCAGTCTC
CGTCATCTATGTATGCAAGCCTCGGCGAGCGAGTGACCATCACGTGCAAGGCGAGT
CAAGATATAAACAGCTACTTGTCATGGTTCCAACAAAAACCAGGGAAATCACCTAA
GACCCTGATCTATAGAGCCAATCGCCTGGTTGACGGTGTCCCCTCCCGCTTTAGCGG
CTCCGGAAGCGGTCAAGATTACTCTCTCACAATTTCTTCCTTGGATTATGAAGACAT
GGGGATCTACTATTGTCAACAGTATGACGAATCCCCGTGGACTTTCGGTGGCGGTAC
CAAATTGGAAATAAAGGGCTCTACAAGCGGCTCAGGAAAACCTGGATCAGGCGAAG
GGTCTACGAAGGGCCAGATACAACTCGTTCAAAGTGGGCCAGAACTCAAAAAACCG
GGAGAAACAGTGAAAATTTCTTGTAAGGCATCAGGATACACATTCACAAACTACGG
GATGAATTGGGTCAAACAAGCACCCGGAAAGGGGCTGCGCTGGATGGGGTGGATCA
ACACACATACTGGGGAACCTACTTACGCAGACGATTTCAAGGGCAGATTCGCCTTTT
CTTTGGAGACCTCCGCCTCTACTGCATACTTGCAGATAAACAACCTGAAGAATGAAG
ATACCGCCACCTACTTCTGTACGCGCAGGGGCTACGATTGGTATTTTGATGTATGGG
GGGCAGGCACCACTGTTACTGTGTCAAGCCGTACGATTGAAGTTATGTATCCTCCTC
CTTACCTAGACAATGAGAAGAGCAATGGAACCATTATCCATGTGAAAGGGAAACAC
CTTTGTCCAAGTCCCCTATTTCCCGGACCTTCTAAGCCCTTTTGGGTGCTGGTGGTGG
TTGGTGGAGTCCTGGCTTGCTATAGCTTGCTAGTAACAGTGGCCTTTATTATTTTCTG
GGTGAGGAGTAAGAGGAGCAGGCTCCTGCACAGTGACTACATGAACATGACTCCCC
GCCGCCCCGGGCCCACCCGCAAGCATTACCAGCCCTATGCCCCACCACGCGACTTCG
CAGCCTATCGCTCAACGCGTGTGAAGTTCAGCAGGAGCGCAGACGCCCCCGCGTAC
CAGCAGGGCCAGAACCAGCTCTATAACGAGCTCAATCTAGGACGAAGAGAGGAGTA
CGATGTTTTGGACAAAAGACGTGGCCGGGACCCTGAGATGGGGGGAAAGCCGAGAA
GGAAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACGAT
GGCCTTTACCAGGGTCTCAGTACAGCCACCAAGGACACCTACGACGCCCTTCACATG
CAGGCCCTGCCCCCTCGCGGACCGCAGTGTACTAATTATGCTCTCTTGAAATTGGCT
GGAGATGTTGAGAGCAATCCCGGGCCCATGCGCATTAGCAAGCCCCACCTGCGGAG
CATCAGCATCCAGTGCTACCTGTGCCTGCTGCTGAACAGCCACTTCCTGACCGAGGC
CGGCATCCACGTGTTCATCCTGGGCTGCTTCAGCGCCGGACTGCCCAAGACCGAGGC
CAACTGGGTGAACGTGATCAGCGACCTGAAGAAGATCGAGGACCTGATCCAGAGCA
TGCACATCGACGCCACCCTGTACACCGAGAGCGACGTGCACCCCAGCTGCAAGGTG
ACCGCCATGAAGTGCTTTCTGCTGGAACTGCAGGTGATCAGCCTGGAAAGCGGCGA
CGCCAGCATCCACGACACCGTGGAGAACCTGATCATCCTGGCCAACAACAGCCTGA
GCAGCAACGGCAACGTGACCGAGAGCGGCTGCAAAGAGTGCGAGGAACTGGAAGA
GAAGAACATCAAAGAGTTTCTGCAGAGCTTCGTGCACATCGTGCAGATGTTCATCAA
CACCAGCTGA

A corresponding amino acid sequence for iC9.CD5CARCD28hinge.28tmd3z.IL15 is as follows:

(SEQ ID NO: 55)
MLEGVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFDVELLKLESG
GGSGVDGFGDVGALESLRGNADLAYILSMEPCGHCLIINNVNFCRESGLRTRTGSNIDCE
KLRRRFSSLHFMVEVKGDLTAKKMVLALLELAQQDHGALDCCVVVILSHGCQASHLQF
PGAVYGTDGCPVSVEKIVNIFNGTSCPSLGGKPKLFFIQACGGEQKDHGFEVASTSPEDE
SPGSNPEPDATPFQEGLRTFDQLDAISSLPTPSDIFVSYSTFPGFVSWRDPKSGSWYVETL
DDIFEQWAHSEDLQSLLLRVANAVSVKGIYKQMPGCFNFLRKKLFFKTSASRAEGRGSL
LTCGDVEENPGPLEMEFGLSWLFLVAILKGVQCSRDIKMTQSPSSMYASLGERVTITCK
ASQDINSYLSWFQQKPGKSPKTLIYRANRLVDGVPSRFSGSGSGQDYSLTISSLDYEDMG
IYYCQQYDESPWTFGGGTKLEIKGSTSGSGKPGSGEGSTKGQIQLVQSGPELKKPGETVK
ISCKASGYTFTNYGMNWVKQAPGKGLRWMGWINTHTGEPTYADDFKGRFAFSLETSA
STAYLQINNLKNEDTATYFCTRRGYDWYFDVWGAGTTVTVSSRTIEVMYPPPYLDNEK
SNGTIIHVKGKHLCPSPLFPGPSKPFWVLVVVGGVLACYSLLVTVAFIIFWVRSKRSRLL
HSDYMNMTPRRPGPTRKHYQPYAPPRDFAAYRSTRVKFSRSADAPAYQQGQNQLYNE
LNLGRREEYDVLDKRRGRDPEMGGKPRRKNPQEGLYNELQKDKMAEAYSEIGMKGER
RRGKGHDGLYQGLSTATKDTYDALHMQALPPRGPQCTNYALLKLAGDVESNPGPMRIS
KPHLRSISIQCYLCLLLNSHFLTEAGIHVFILGCFSAGLPKTEANWVNVISDLKKIEDLIQS
MHIDATLYTESDVHPSCKVTAMKCFLLELQVISLESGDASIHDTVENLIILANNSLSSNGN
VTESGCKECEELEEKNIKEFLQSFVHIVQMFINTS

### C. T Cell Receptor (TCR)

In some embodiments, a CD5-targeting genetically engineered antigen receptor includes recombinant TCRs and/or TCRs cloned from naturally occurring T cells. A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable ß chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### II. Cytokines

One or more cytokines may be utilized with one or more CD5-targeting genetically engineered receptors, such as CD5-specific CARs. In some cases, one or more cytokines are present on the same vector molecule as the engineered receptor, although in other cases they are on separate vector molecules. In particular embodiments, one or more cytokines are co-expressed from the same vector as the engineered receptor. One or more cytokines may be produced as a separate polypeptide from the CD5-specific receptor. As one example, Interleukin-15 (IL-15), is utilized. IL-15 may be employed because, for example, it is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death. In addition to IL-15, other cytokines are envisioned. These include, but are not limited to, cytokines, chemokines, and other molecules that contribute to the activation and proliferation of cells used for human application. As one example, the cytokine is IL-15, IL-12, IL-2, IL-18, IL-21, IL-7, or combination thereof. NK cells expressing IL-15 may be utilized and are capable of continued supportive cytokine signaling, which is useful for their survival post-infusion.

In specific embodiments, NK cells express one or more exogenously provided cytokines. The cytokine may be exogenously provided to the NK cells because it is expressed from an expression vector within the cell and/or because it is provided in a culture medium of the cells. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide. The cytokine may be expressed as a separate polypeptide molecule as a suicide gene and as a separate polypeptide from an engineered receptor of the cell. In some embodiments, the present disclosure concerns co-utilization of CAR and/or TCR vectors with IL-15, particularly in NK cells.

### III. Suicide Genes

In particular embodiments, a suicide gene is utilized in conjunction with cell therapy of any kind to control its use and allow for termination of the cell therapy at a desired event and/or time. The suicide gene is employed in transduced cells for the purpose of eliciting death for the transduced cells when needed. The CD5-targeting cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes also include CD20, CD52, EGFRv3, or inducible caspase 9. In one embodiment, a truncated version of EGFR variant III (EGFRv3) may be used as a suicide antigen that can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

In particular embodiments, vectors that encode the CD5-targeting CAR, or any vector in a NK cell encompassed herein, include one or more suicide genes. The suicide gene may or may not be on the same vector as a CD5-targeting CAR. In cases wherein the suicide gene is present on the same vector as the CD5-targeting CAR, the suicide gene and the CAR may be separated by an IRES or 2A element, for example.

### IV. Vectors

The CD5-targeting CARs may be delivered to the recipient immune cells by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the immune cell is transduced with a vector encoding the CD5-targeting receptor and also requires transduction of another gene or genes into the cell, such as a suicide gene and/or cytokine and/or an optional therapeutic gene product, the CD5-targeting receptor, suicide gene, cytokine, and optional therapeutic gene may or may not be comprised on or with the same vector. In some cases, the CD5-targeting CAR, suicide gene, cytokine, and optional therapeutic gene are expressed from the same vector molecule, such as the same viral vector molecule. In such cases, the expression of the CD5-targeting CAR, suicide gene, cytokine, and optional therapeutic gene may or may not be regulated by the same regulatory element(s). When the CD5-targeting CAR, suicide gene, cytokine, and optional therapeutic gene are on the same vector, they may or may not be expressed as separate polypeptides. In cases wherein they are expressed as separate polypeptides, they may be separated on the vector by a 2A element or IRES element (or both kinds may be used on the same vector once or more than once), for example.

### A. General Embodiments

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

### 1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

### 2. Promoter/Enhancers

The expression constructs provided herein comprise a promoter to drive expression of the antigen receptor and other cistron gene products. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### 3. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

### 4. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

### 5. Selection and Screenable Markers

In some embodiments, NK cells comprising a CD5-targeting receptor construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### B. Multicistronic Vectors

In particular embodiments, the CD5-targeting receptor, optional suicide gene, optional cytokine, and/or optional therapeutic gene are expressed from a multicistronic vector (The term “cistron” as used herein refers to a nucleic acid sequence from which a gene product may be produced). In specific embodiments, the multicistronic vector encodes the CD5-targeting receptor, the suicide gene, and at least one cytokine, and/or engineered receptor, such as a T-cell receptor and/or an additional non-CD5-targeting CAR. In some cases, the multicistronic vector encodes at least one CD5-targeting CAR, at least one TNF-alpha mutant, and at least one cytokine. The cytokine may be of a particular type of cytokine, such as human or mouse or any species. In specific cases, the cytokine is IL15, IL 12, IL2, IL18, and/or IL21.

In certain embodiments, the present disclosure provides a flexible, modular system (the term “modular” as used herein refers to a cistron or component of a cistron that allows for interchangeability thereof, such as by removal and replacement of an entire cistron or of a component of a cistron, respectively, for example by using standard recombination techniques) utilizing a polycistronic vector having the ability to express multiple cistrons at substantially identical levels. The system may be used for cell engineering allowing for combinatorial expression (including overexpression) of multiple genes. In specific embodiments, one or more of the genes expressed by the vector includes one, two, or more antigen receptors. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise: (1) one or more reporters, for example fluorescent or enzymatic reporters, such as for cellular assays and animal imaging; (2) one or more cytokines or other signaling molecules; and/or (3) a suicide gene.

In specific cases, the vector may comprise at least 4 cistrons separated by cleavage sites of any kind, such as 2A cleavage sites. The vector may or may not be Moloney Murine Leukemia Virus (MoML V or MMLV)-based including the 3′ and 5′ LTR with the psi packaging sequence in a pUC19 backbone. The vector may comprise 4 or more cistrons with three or more 2A cleavage sites and multiple ORFs for gene swapping. The system allows for combinatorial overexpression of multiple genes (7 or more) that are flanked by restriction site(s) for rapid integration through subcloning, and the system also includes at least three 2A self-cleavage sites, in some embodiments. Thus, the system allows for expression of multiple CARs, TCRs, signaling molecules, cytokines, cytokine receptors, and/or homing receptors. This system may also be applied to other viral and non-viral vectors, including but not limited lentivirus, adenovirus AAV, as well as non-viral plasmids.

The modular nature of the system also enables efficient subcloning of a gene into each of the 4 cistrons in the polycistronic expression vector and the swapping of genes, such as for rapid testing. Restriction sites strategically located in the polycistronic expression vector allow for swapping of genes with efficiency.

Embodiments of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has embodiments wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

Embodiments of the disclosure include polycistronic vectors comprising at least two, at least three, or at least four cistrons each flanked by one or more restriction enzyme sites, wherein at least one cistron encodes for at least one antigen receptor. In some cases, two, three, four, or more of the cistrons are translated into a single polypeptide and cleaved into separate polypeptides, whereas in other cases multiple of the cistrons are translated into a single polypeptide and cleaved into separate polypeptides. Adjacent cistrons on the vector may be separated by a self cleavage site, such as a 2A self cleavage site. In some cases each of the cistrons express separate polypeptides from the vector. On particular cases, adjacent cistrons on the vector are separated by an IRES element.

In certain embodiments, the present disclosure provides a system for cell engineering allowing for combinatorial expression, including overexpression, of multiple cistrons that may include one, two, or more antigen receptors, for example. In particular embodiments, the use of a polycistronic vector as described herein allows for the vector to produce equimolar levels of multiple gene products from the same mRNA. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise one or more fluorescent or enzymatic reporters, such as for cellular assays and animal imaging. The vector may also comprise a suicide gene product for termination of cells harboring the vector when they are no longer needed or become deleterious to a host to which they have been provided.

In specific embodiments, the vector is a viral vector (retroviral vector, lentiviral vector, adenoviral vector, or adeno-associated viral vector, for example) or a non-viral vector. The vector may comprise a Moloney Murine Leukemia Virus (MMLV) 5′ LTR, 3′ LTR, and/or psi packaging element. In specific cases, the psi packaging is incorporated between the 5′ LTR and the antigen receptor coding sequence. The vector may or may not comprise pUC19 sequence. In some aspects of the vector, at least one cistron encodes for a cytokine (interleukin 15 (IL-15), IL-7, IL-21, IL-18, IL-12, or IL-2, for example), chemokine, cytokine receptor, and/or homing receptor.

When 2A cleavages sites are utilized in the vector, the 2A cleavage site may comprise a P2A, T2A, E2A and/or F2A site.

A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

Exemplary vectors may be circular and by convention, where position 1 (12 o'clock position at the top of the circle, with the rest of the sequence in clock-wise direction) is set at the start of 5′ LTR.

In embodiments wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (Thosea asigna virus 2A) were also identified. The mechanism of 2A-mediated “self-cleavage” was discovered to be ribosome skipping the formation of a glycyl-prolyl peptide bond at the C-terminus of the 2A.

In specific cases, the vector may be a γ-retroviral transfer vector. The retroviral transfer vector may comprises a backbone based on a plasmid, such as the pUC19 plasmid (large fragment (2.63 kb) in between HindIII and EcoRI restriction enzyme sites). The backbone may carry viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, brackets the genetic cargo of interest, such as CD5-targeting CARs and associated components. The psi packaging sequence, which is a target site for packaging by nucleocapsid, is also incorporated in cis, sandwiched between the 5′ LTR and the CAR coding sequence. Thus, the basic structure of an example of a transfer vector can be configured as such: pUC19 sequence-5′ LTR-psi packaging sequence-genetic cargo of interest-3′ LTR-pUC19 sequence. This system may also be applied to other viral and non-viral vectors, including but not limited lentivirus, adenovirus AAV, as well as non-viral plasmids.

### V. Cells

The present disclosure encompasses immune cells or stem cells of any kind that harbor at least one vector that encodes a CD5-targeting receptor and that also may encode at least one cytokine and/or at least one suicide gene. In some cases, different vectors encode the CAR vs. encodes the suicide gene and/or cytokine. The immune cells, including NK cells, may be derived from cord blood (including pooled cord blood from multiple sources), peripheral blood, induced pluripotent stem cells (iPSCs), hematopoietic stem cells (HSCs), bone marrow, or a mixture thereof. The NK cells may be derived from a cell line such as, but not limited to, NK-92 cells, for example. The NK cell may be a cord blood mononuclear cell, such as a CD56+NK cell.

The present disclosure encompasses immune or other cells of any kind, including conventional T cells, gamma-delta T cells, NKT and invariant NK T cells, regulatory T cells, macrophages, B cells, dendritic cells, mesenchymal stromal cells (MSCs), or a mixture thereof.

In some cases, the cells have been expanded in the presence of an effective amount of universal antigen presenting cells (UAPCs), including in any suitable ratio. The cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example. In some cases, the NK cells were expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

Following genetic modification with the vector(s), the NK cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the CD5-targeting CAR is expanded ex vivo. The clone selected for expansion demonstrates the capacity to specifically recognize and lyse CD5-expressing target cells. The recombinant immune cells may be expanded by stimulation with IL-2, or other cytokines that bind the common gamma-chain (e.g., IL-7, IL-12, IL-15, IL-18, IL-21, IL-23, and others). The recombinant immune cells may be expanded by stimulation with artificial antigen presenting cells. In a further aspect, the genetically modified cells may be cryopreserved.

Embodiments of the disclosure encompass cells that express one or more CD5-targeting CARs and one or more suicide genes as encompassed herein. The NK cell comprises a recombinant nucleic acid that encodes one or more CD5-targeting CARs and one or more engineered nonsecretable, membrane bound TNF-alpha mutant polypeptides, in specific embodiments. In specific embodiments, in addition to expressing one or more CD5-targeting CARs and TNF-alpha mutant polypeptides, the cell also comprises a nucleic acid that encodes one or more therapeutic gene products.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to express the CD5-targeting CAR, optional suicide gene, optional cytokine(s), and optional therapeutic gene product(s) (using standard techniques for transduction and expansion for adoptive cell therapy, for example), they may be provided back to the individual from which they were originally sourced. In some cases, the cells are stored for later use for the individual or another individual.

The immune cells may be comprised in a population of cells, and that population may have a majority that are transduced with one or more CD5-targeting receptors and/or one or more suicide genes and/or one or more cytokines. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of immune cells that are transduced with one or more CD5-targeting receptors and/or one or more suicide genes and/or one or more cytokines. The one or more CD5-targeting receptors and/or one or more suicide genes and/or one or more cytokines may be separate polypeptides.

The immune cells may be produced with the one or more CD5-targeting receptors and/or one or more suicide genes and/or one or more cytokines for the intent of being modular with respect to a specific purpose. For example, cells may be generated, including for commercial distribution, expressing a CD5-targeting CARs and/or one or more suicide genes and/or one or more cytokines (or distributed with a nucleic acid that encodes the mutant for subsequent transduction), and a user may modify them to express one or more other genes of interest (including therapeutic genes) dependent upon their intended purpose(s). For instance, an individual interested in treating CD5-positive cells, including CD5-positive cancer, may obtain or generate suicide gene-expressing cells (or heterologous cytokine-expressing cells) and modify them to express a receptor comprising a CD5-specific scFv, or vice versa.

In particular embodiments, NK cells are utilized, and the genome of the transduced NK cells expressing the one or more CD5-targeting CARs and/or one or more suicide genes and/or one or more cytokines may be modified. The genome may be modified in any manner, but in specific embodiments the genome is modified by CRISPR gene editing, for example. The genome of the cells may be modified to enhance effectiveness of the cells for any purpose.

### VI. Gene Editing of CD5-specific CAR Cells

In particular embodiments, cells comprising at least a CD5-specific engineered receptor are gene edited to modify expression of one or more endogenous genes in the cell. In specific cases, the CD5-specific CAR cells are modified to have reduced levels of expression of one or more endogenous genes, including inhibition of expression of one or more endogenous genes (that may be referred to as knocked out). Such cells may or may not be expanded.

In particular cases, one or more endogenous genes of the CD5-specific CAR cells are modified, such as disrupted in expression where the expression is reduced in part or in full. In specific cases, one or more genes are knocked down or knocked out using processes of the disclosure. In specific cases, multiple genes are knocked down or knocked out, and this may or may not occur in the same step in their production. The genes that are edited in the CD5-specific CAR cells may be of any kind, but in specific embodiments the genes are genes whose gene products inhibit activity and/or proliferation of the CD5-specific CAR cells, including CD5-specific CAR NK cells, such as those derived from cord blood, as one example. In specific cases the genes that are edited in the CD5-specific CAR cells allow the CD5-specific CAR cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, CD3δ, CREM, IL10R, CD5, and CD7. In specific embodiments, the TGFBR2 gene is knocked out or knocked down in the CD5-specific CAR cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins; in some embodiments, CpF1 is utilized instead of Cas9. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). In some cases, CpF1 may be used as an endonuclease instead of Cas9. The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### VII. Methods of Treatment

In various embodiments, diseased or other cells expressing endogenous CD5 on their surface are targeted for the purpose of improving a medical condition in an individual that has the medical condition or for the purpose of reducing the risk or delaying the severity and/or onset of the medical condition in an individual. In specific cases, cancer cells expressing endogenous CD5 are targeted for the purpose of killing the cancer cells.

CD5-targeting CAR constructs, nucleic acid sequences, vectors, immune cells and so forth as contemplated herein, and/or pharmaceutical compositions comprising the same, are used for the prevention, treatment or amelioration of a cancerous disease, such as a tumorous disease. In particular embodiments, the pharmaceutical composition of the present disclosure may be particularly useful in preventing, ameliorating and/or treating cancer, including cancers that express CD5 and that may or may not be solid tumors, for example.

The immune cells for which the CD5-targeting receptor is utilized may be NK, T cells, gamma delta T cells, or NKT or invariant NKT (iNKT), or invariant NKT cells engineered for cell therapy for mammals, in particular embodiments. In such cases where the cells are NK cells, the NK cell therapy may be of any kind and the NK cells may be of any kind. In specific embodiments, the cells are NK cells that have been engineered to express one or more CD5-targeting CARs and/or one or more suicide genes and/or one or more cytokines. In specific embodiments, the cells are NK cells that are transduced with a CD5-targeting CAR.

In particular embodiments, the present disclosure contemplates, in part, CD5 CAR-expressing cells, CD5-targeting CAR constructs, CD5-targeting CAR nucleic acid molecules and CD5-targeting CAR vectors that can be administered either alone or in any combination using standard vectors and/or gene delivery systems, and in at least some aspects, together with a pharmaceutically acceptable carrier or excipient. In certain embodiments, subsequent to administration, the nucleic acid molecules or vectors may be stably integrated into the genome of the subject.

In specific embodiments, viral vectors may be used that are specific for certain cells or tissues and persist in NK cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject in the need thereof an effective amount of cells that express a CD5-targeting CAR, a nucleic acid sequence, a vector, as contemplated herein and/or produced by a process as contemplated herein.

Possible indications for administration of the composition(s) of the exemplary CD5-targeting CAR cells are cancerous diseases, including tumorous diseases, including B cell malignancies, multiple myeloma, breast cancer, glioblastoma, renal cancer, pancreatic cancer, or lung cancer, for example. Exemplary indications for administration of the composition(s) of CD5-targeting CAR cells are cancerous diseases, including any malignancies that express CD5. The administration of the composition(s) of the disclosure is useful for all stages (I, II, III, or IV) and types of cancer, including for minimal residual disease, early cancer, advanced cancer, and/or metastatic cancer and/or refractory cancer, for example.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising a CD5-targeting CAR construct as defined herein, a nucleic acid sequence as defined herein, a vector as defined herein and/or a host cell (such as an immune cell) as defined herein. It is also contemplated that the kit of this disclosure comprises a pharmaceutical composition as described herein above, either alone or in combination with further medicaments to be administered to an individual in need of medical treatment or intervention.

### A. Pharmaceutical Compositions

Also provided herein are pharmaceutical compositions and formulations comprising transduced NK cells and a pharmaceutically acceptable carrier. The transduced cells may be comprised in a media suitable for transfer to an individual and/or media suitable for preservation, such as cryopreservation, including prior to transfer to an individual.

Pharmaceutical compositions and formulations as described herein can be prepared by mixing the active ingredients (such as the cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### B. Combination Therapies

In certain embodiments, the compositions and methods of the present embodiments involve an immune cell population (including NK cell population) in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, oncolytic viruses, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

In particular embodiments, in addition to the inventive cell therapy of the disclosure, the individual may have been provided, may be provided, and/or may will be provided a specific additional therapy for cancer, including one or more of surgery, radiation, immunotherapy (other than the cell therapy of the present disclosure), hormone therapy, gene therapy, chemotherapy, and so forth.

An immune cell therapy may be administered before, during, after, or in various combinations relative to an additional cancer therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons □, □□ and □, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VIII. Kits of the Disclosure

Any of the compositions described herein may be comprised in a kit. In a non-limiting example, cells, reagents to produce cells, vectors, and reagents to produce vectors and/or components thereof may be comprised in a kit. In certain embodiments, NK cells may be comprised in a kit, and they may or may not yet express a CD5-targeting receptor, an optional cytokine, or an optional suicide gene. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode one or more CD5-targeting CARs, suicide gene products, and/or cytokines may be included in the kit. Proteins, such as cytokines or antibodies, including monoclonal antibodies, may be included in the kit. Nucleotides that encode components of engineered CAR receptors may be included in the kit, including reagents to generate same.

In particular aspects, the kit comprises the NK cell therapy of the disclosure and also another cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

### IX. Examples

The following examples are included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### Example 1

### DAP10 Co-Stimulation Imparts Memory-Like Features to CD5 Targeting Cord Blood-Derived CAR-NK Cells

The present example concerns analysis of a variety of CD5 chimeric antigen receptor (CAR)-related constructs for use in adoptive cell therapy for the treatment of CD5+ cancer cells.

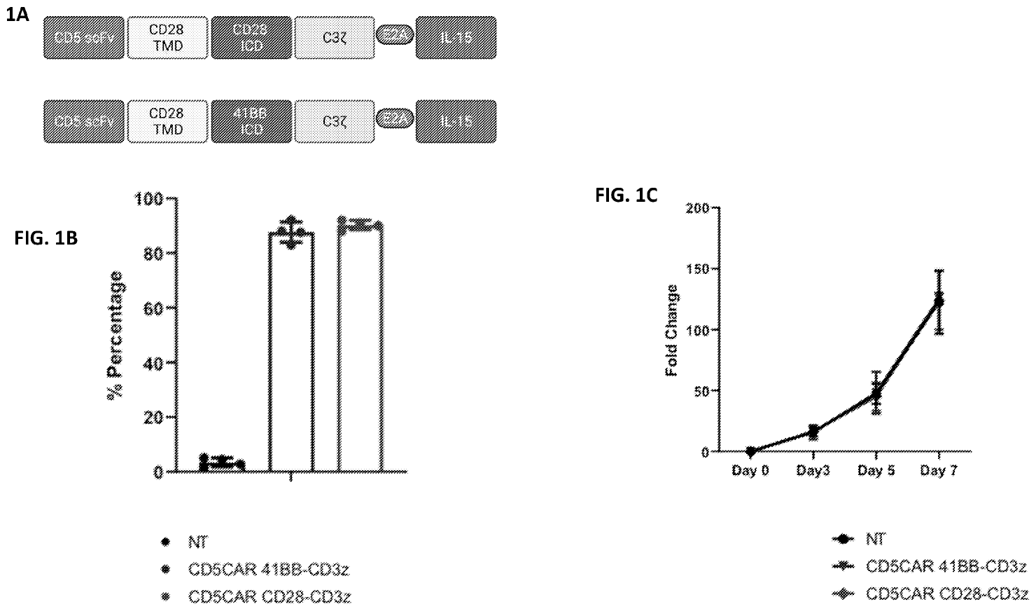

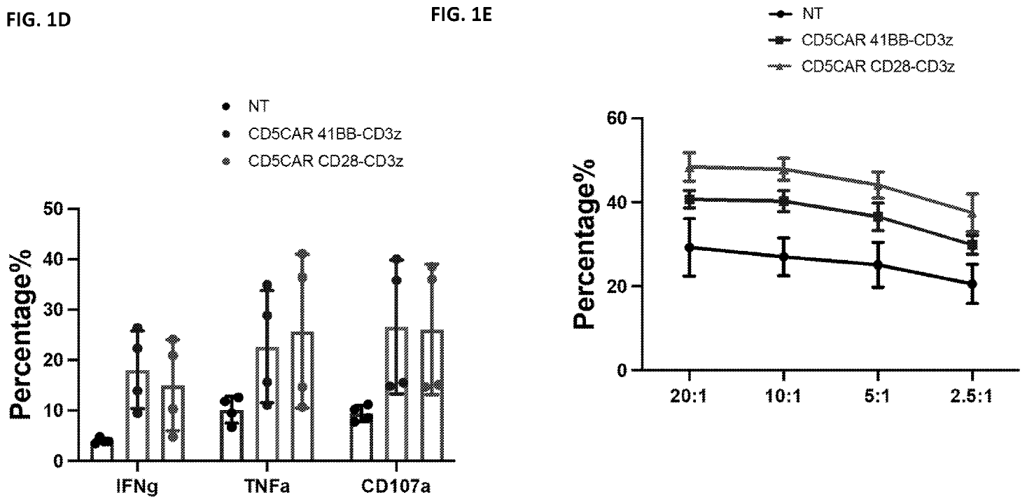

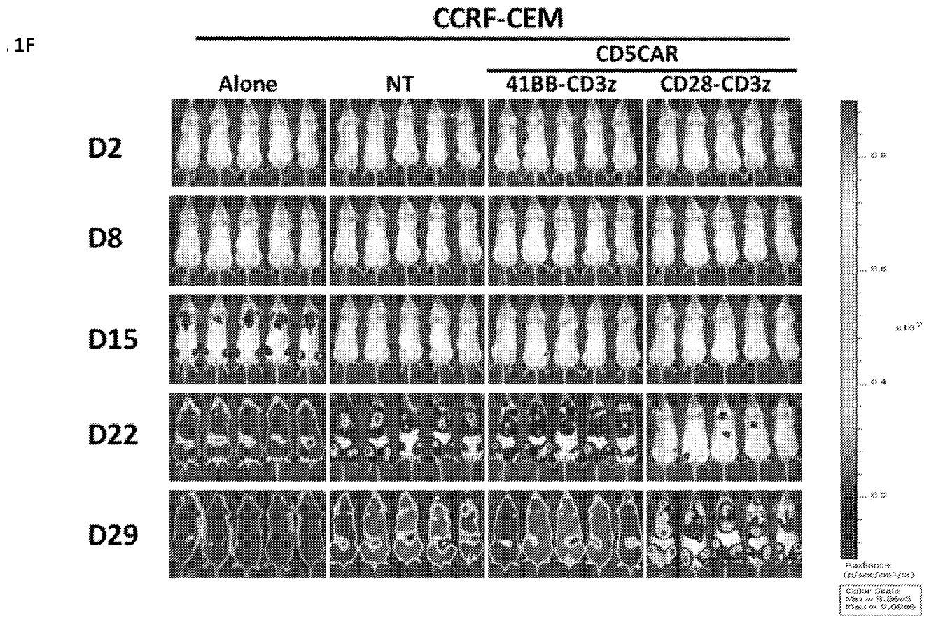

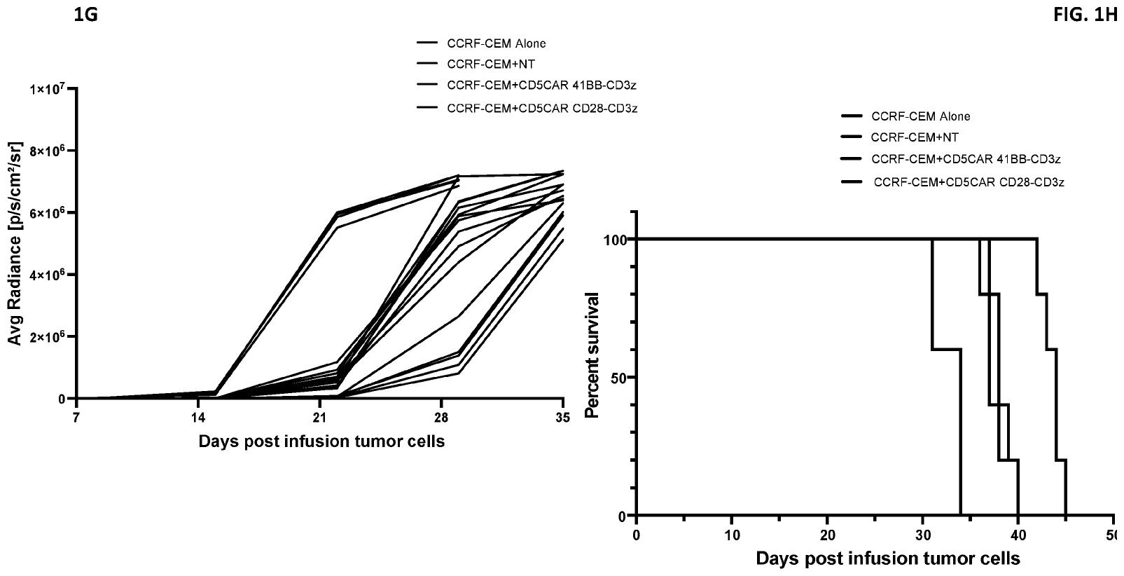

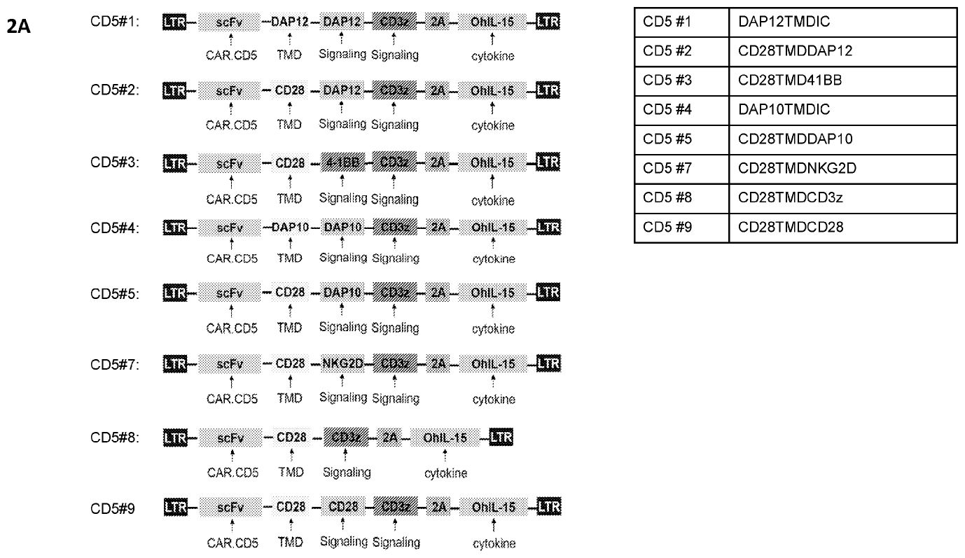

Testing T-cell centric co-stimulatory domains CD28 or 4-1BB in the design of CD5 CAR-NK cells. The efficacy of CD5 CAR-NK cells incorporating CD28 or 4-1BB as co-stimulatory domains in CAR constructs was characterized. Two CD5 CAR constructs with an scFv directed against the CD5 antigen was produced having either CD28 or 4-1BB co-stimulatory domain, CD3ζ signaling domain and IL-15 as a cytokine to enhance in vivo proliferation and persistence (FIG. 1A). Cord blood (CB) derived NK cells were successfully transduced with these constructs (FIG. 1B) and demonstrated good proliferation without evidence of fratricide (FIG. 1C). These CD5 CAR-NK cells also demonstrated enhanced cytokine production (IFNγ, TNFα), degranulation (CD107a) and cytotoxicity compared to non-transduced (NT) NK cells against CCRF-CEM, a CD5+ T-cell acute lymphoblastic leukemia (T-ALL) cell line (FIG. 1. D,E). In an in vivo mouse model of T-ALL, mice engrafted with CCRF-CEM transduced with fireflyluciferase (CCRF-CEM-FFluc) and treated with CD5-CD28-CD3ζ-IL-15 or CD5-41BB-CD3ζ-IL-15 CAR-NK cells showed improved tumor control and survival compared to mice engrafted with tumor alone or mice engrafted with tumor and treated with NT NK cells (FIG. 1F-1H), however all the mice eventually succumbed to tumor.

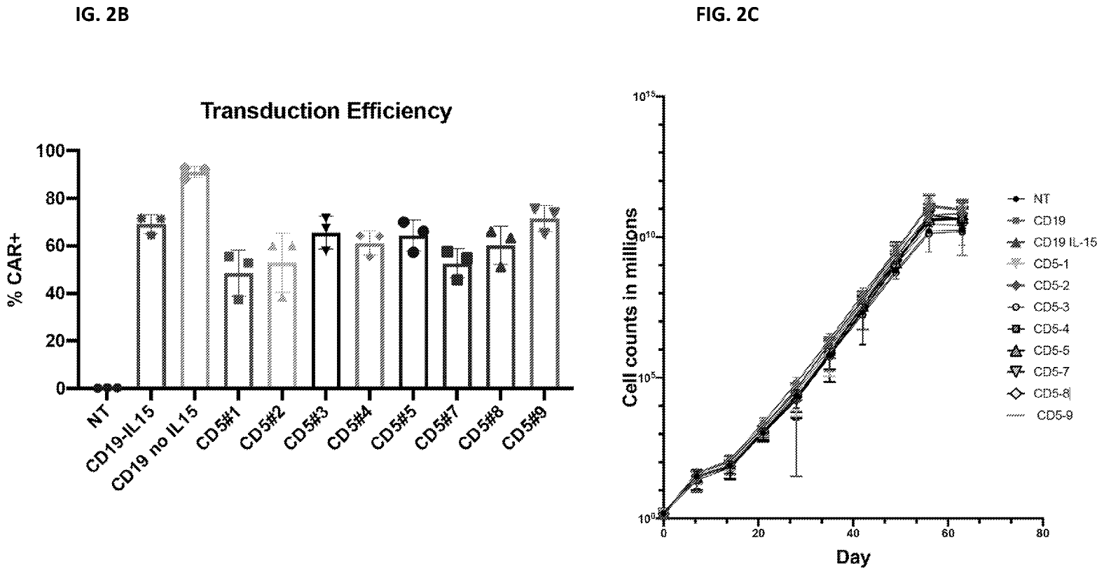

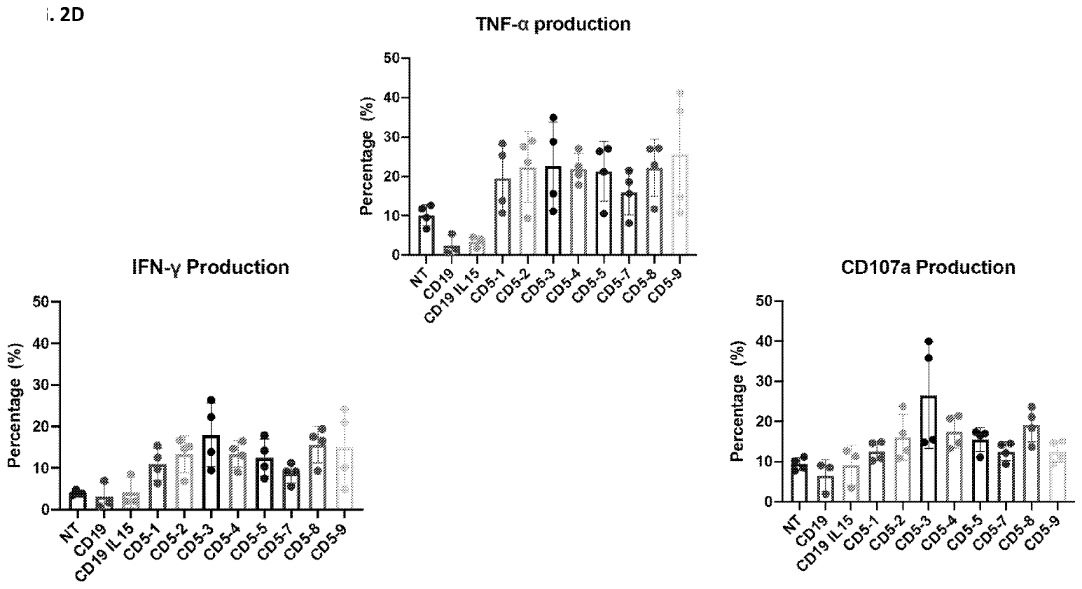

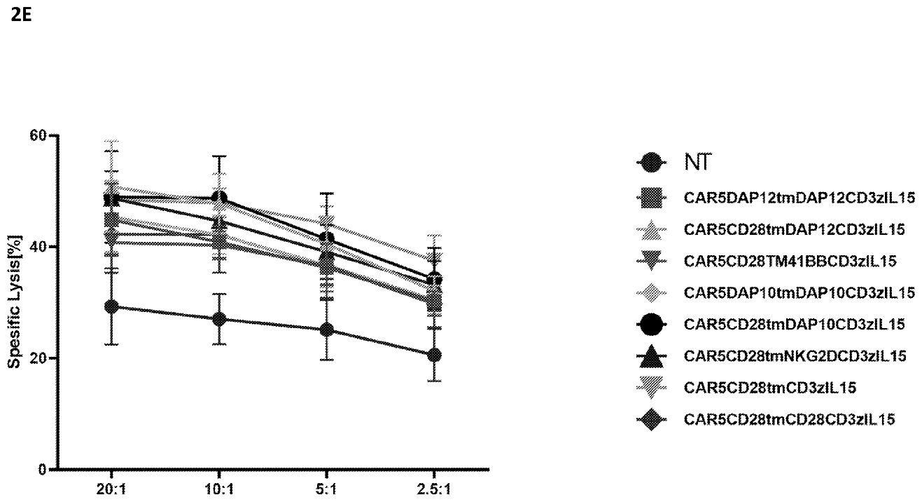

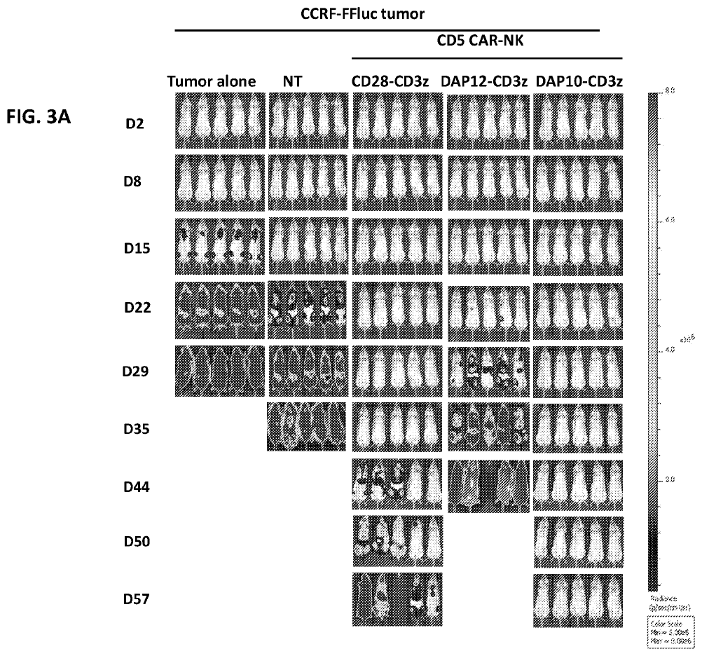

Evaluating the role of NK relevant co-stimulatory domains in the in vitro function of CD5 CAR-NK cells. Given the fact that CD28 and 4-1BB are T-cell centric co-stimulatory domains and that CD28 is not expressed in non-engineered NK cells, it was considered that incorporating co-stimulatory molecules that are more relevant to NK cell biology and signaling would enhance the efficacy of CD5 CAR-NK cells, in specific embodiments. Three additional retroviral constructs were designed; the constructs had in common the same scFv against CD5, CD28 transmembrane (TM) domain, CD3ζ signaling domain and IL-15 as a cytokine to enhance in vivo persistence and proliferation, but differed in the choice of co-stimulatory domain (NKG2D vs DAP10 vs DAP12) (FIG. 2A). For the constructs with DAP10 and DAP12 co-stimulatory domains, designs with DAP10 and DAP12 TM domain were also included (FIG. 2A). A first-generation CAR construct without the inclusion of a co-stimulatory domain was used as negative control to dissect the role of co-stimulation in the function of CAR-NK cells. CD5-CD28-CD3ζ-IL-15 CAR-NK cells served as a positive control with T-cell centric co-stimulation since they demonstrated in vitro and in vivo efficacy against CD5+T-ALL targets. By adjusting the multiplicity of infection (MOI), similar transduction efficiency was obtained (50-60%) for all the tested constructs (FIG. 2B). The proliferation of the various CD5 CAR-NK cells was very similar in vitro since the expansion protocol includes universal antigen presenting cells (uAPCs) and IL-2 as previously described,¹² and optimizes the proliferation of NK cells regardless of their engineering (FIG. 2C). All CD5 CAR-NK cells regardless of the CAR design demonstrated enhanced cytokine production (IFNγ and TNFα) and degranulation (CD107a) when co-cultured with CD5+CCRF-CEM tumor cells for 6 hrs compared to control NT NK cells and CD19 CAR-NK cells (with or without IL-15) used as irrelevant target CAR control (FIG. 2D). In a 51Cr release cytotoxicity assay, all CD5 CAR-NK cells killed CD5+ tumor targets better than NT NK cells, however, no significant differences in cytotoxicity were discerned among the various CAR-NK cell designs (FIG. 2E).

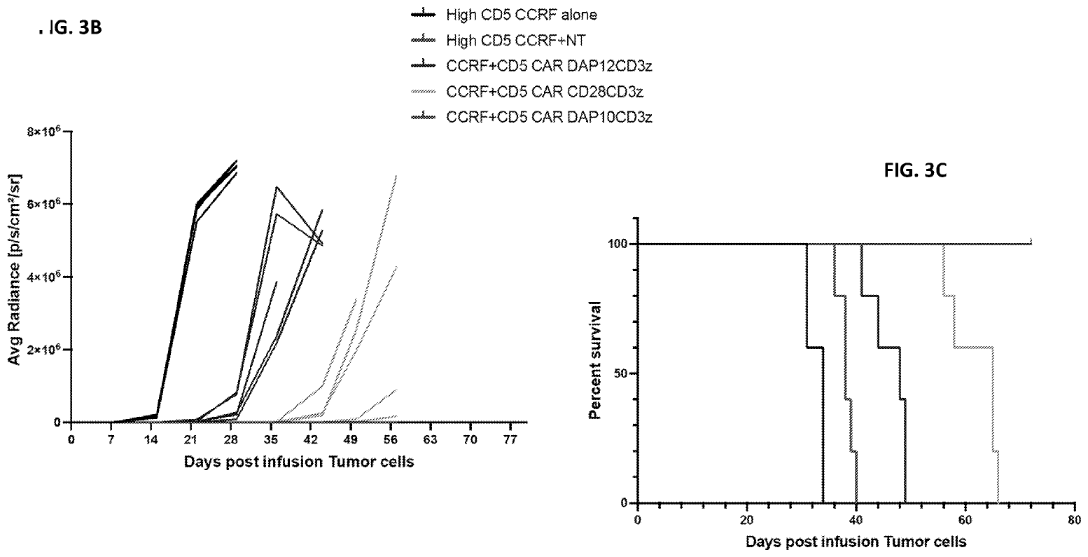

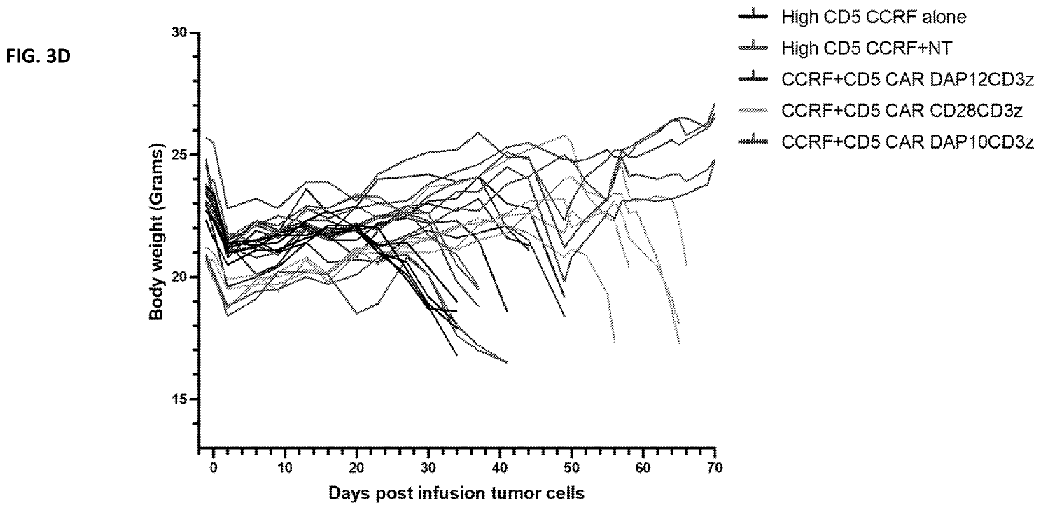

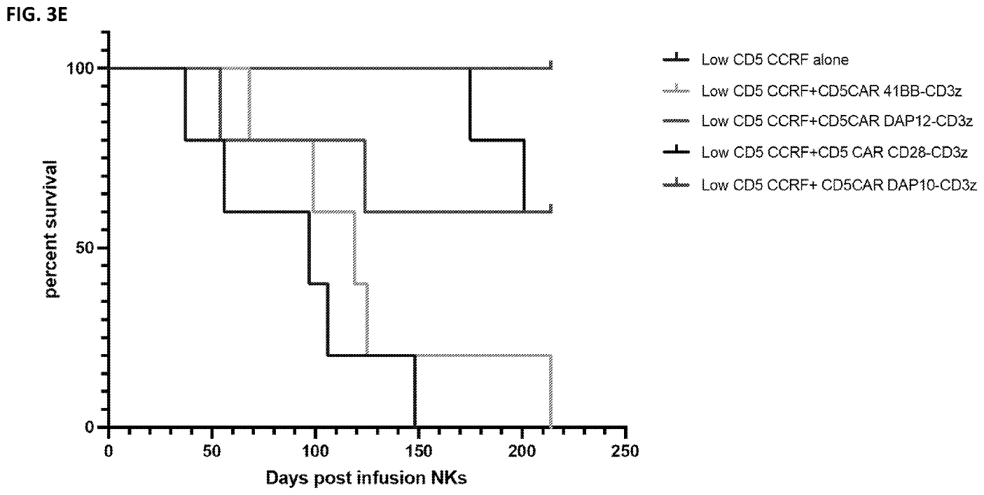

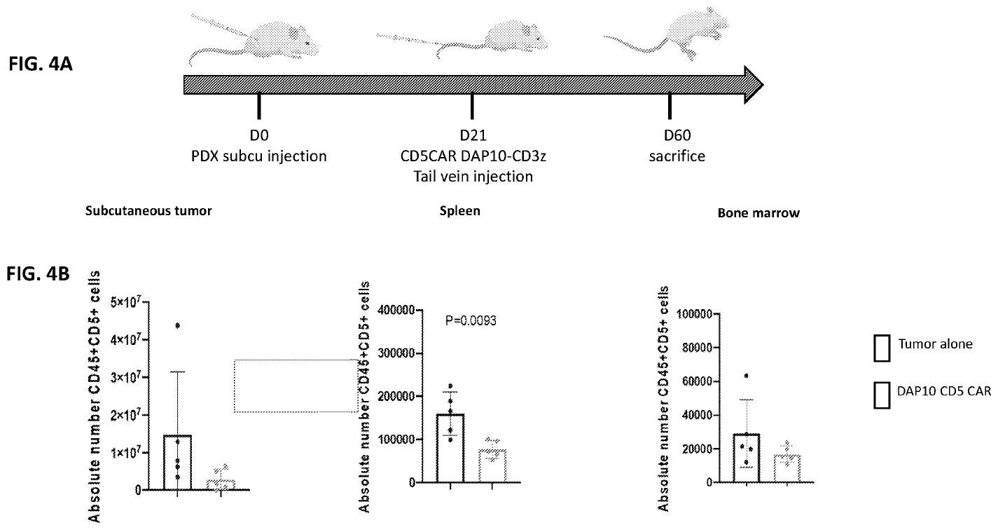

CD5 CAR-NK cells with DAP10 co-stimulation led to enhanced tumor control and survival in mice models of CD5+ malignancies. To investigate the in vivo anti-tumor activity of the various CD5 CAR-NK cell designs, the same NSG mouse model was used of CD5+CCRF-CEM used earlier to test the in vivo efficacy of CD5-CD28-CD3ζ and CD5-41BB-CD3ζ CD5 CAR-NK cells. Mice engrafted with tumor alone and mice engrafted with tumor and treated with NT NK cells served as negative controls. Mice were irradiated (225cGy) on day −1 and then injected intravenously with CCRF-CEM-FFluc tumor 100,000 cells per mouse on day 0. On day 2, the treatment groups received either 2×10⁶ NT NK cells or 2×10⁶ of the various preparations of CAR-NK cells. CD5 CAR-NK cells with DAP10 signaling showed clearly superior tumor control and significant improvement in survival compared to control mice and mice that received CD5 CAR-NK cells incorporating other co-stimulatory domains (FIG. 3A-C). Furthermore, animals treated with these CAR-NK cells showed no signs of toxicity as evidenced by body weight monitoring (no weight loss) (FIG. 3D). Interestingly, the survival advantage afforded by CD5 CAR-NK cells with DAP10 co-stimulation was maintained even in mice engrafted with CCRF-CEM with low CD5 expression (FIG. 3E). Furthermore, in an in vivo PDX mouse model of CD5+ mantle cell lymphoma injected subcutaneously, CD5 CAR-NK cells with DAP10 co-stimulation led to improvement in tumor control with a notable decrease in the absolute number of CD45+CD5+ lymphoma cells from the subcutaneous tumor, spleen and bone marrow of the CAR-NK cell treated mice compared to untreated mice engrafted with tumor alone (FIG. 4A,4B).

### DAP10 Co-Stimulation Imparts an Activated Phenotype to CD5 CAR-NK Cells.

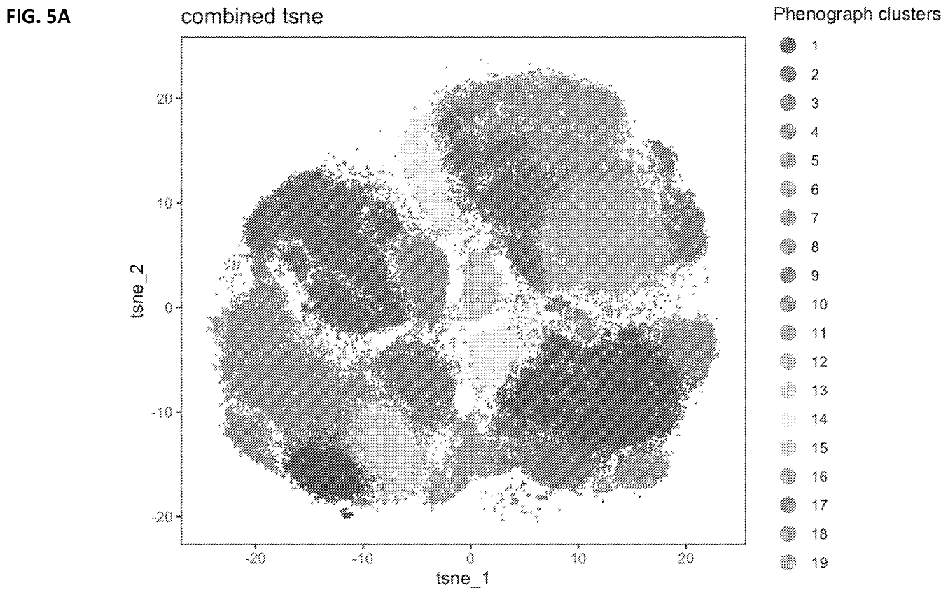

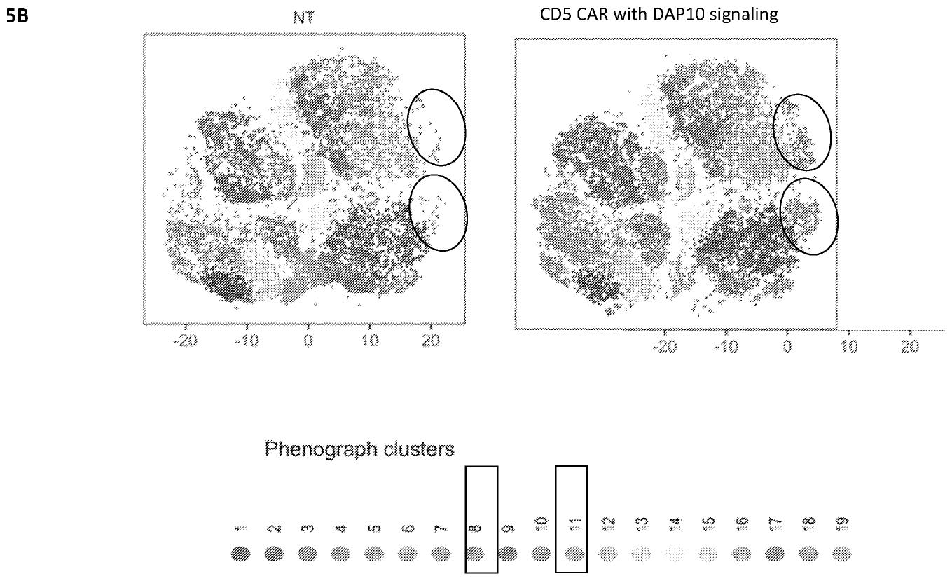

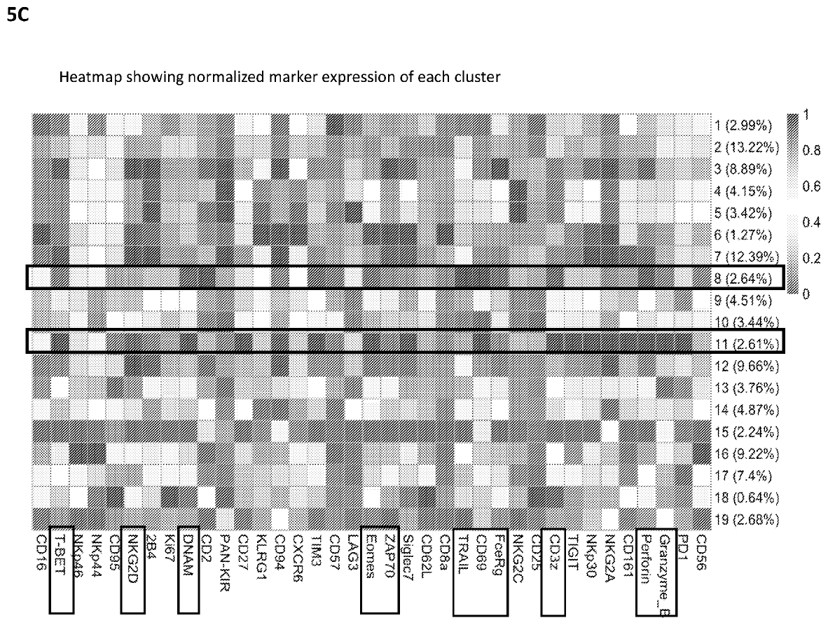

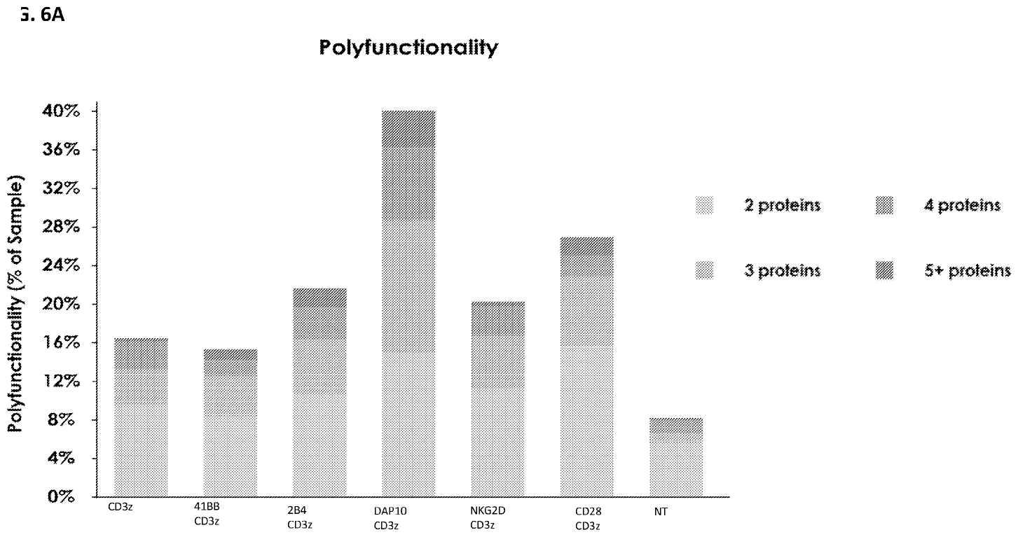

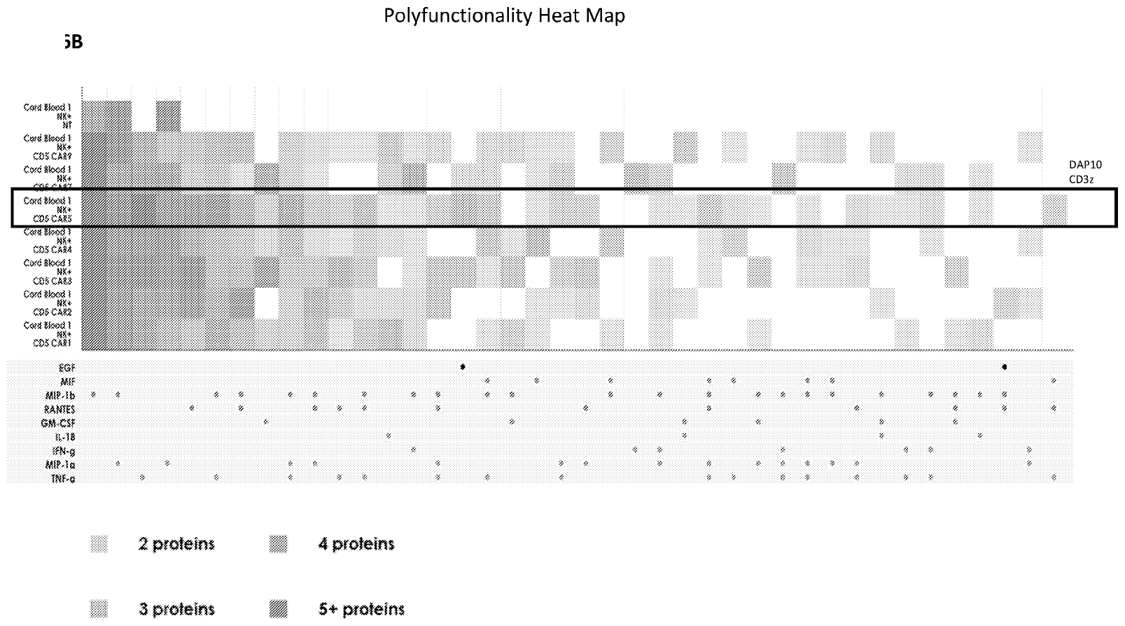

To characterize why CD5 CAR-NK cells with DAP10 co-stimulatory domain perform remarkably better in vivo compared to other CAR designs, a deeper look was taken at their proteomic profile. To investigate the phenotype of the various CD5 CAR-NK cells in reference to NT NK cells, mass cytometry was used and a panel of 47 antibodies against activating and inhibitory NK cell receptors, as well as proliferation, differentiation and maturation markers. Using viSNE, a t-distributed stochastic neighbor-embedding (t-SNE) algorithm, it was observed that CD5 CAR-NK cells with DAP10 co-stimulatory domain demonstrated two specific clusters (Clusters 8 and 11) compared to NT NK cells (FIG. 5A,5B). By taking a closer look at the combination of markers defining each cluster, these clusters 8 and 11 were enriched in activation markers (NKG2D, DNAM, CD69, CD3ζ), cytotoxicity markers (perforin, granzyme B, TRAIL) and maturation markers (eomesodermin (eomes) and T-bet) (FIG. 5C).

CD5 CAR-NK Cells with DAP10 Co-Stimulatory Domain Demonstrate Enhanced Polyfunctionality and Metabolic Fitness.

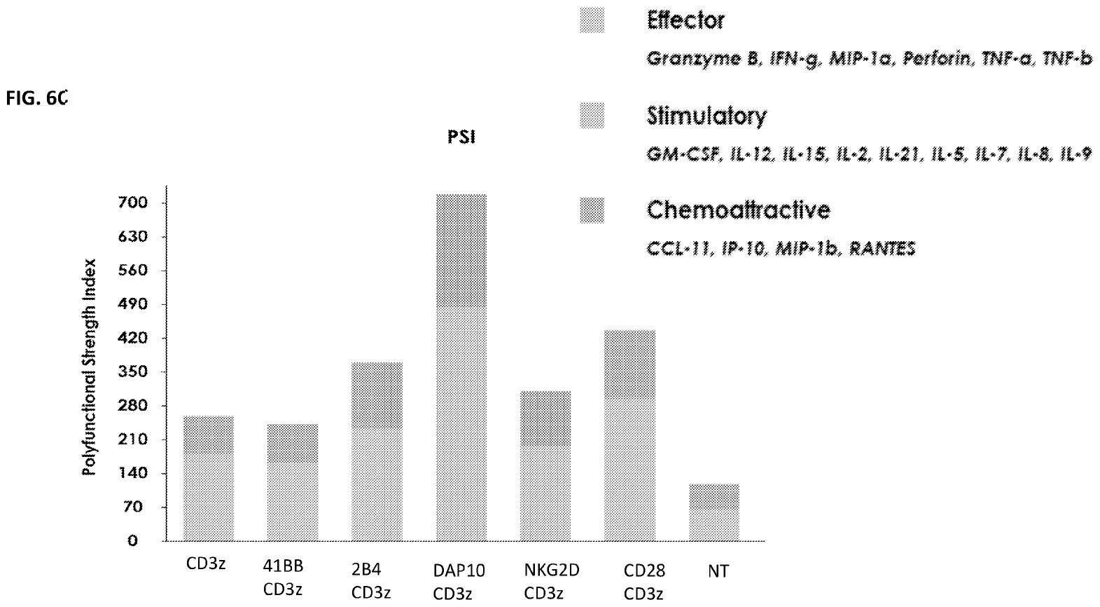

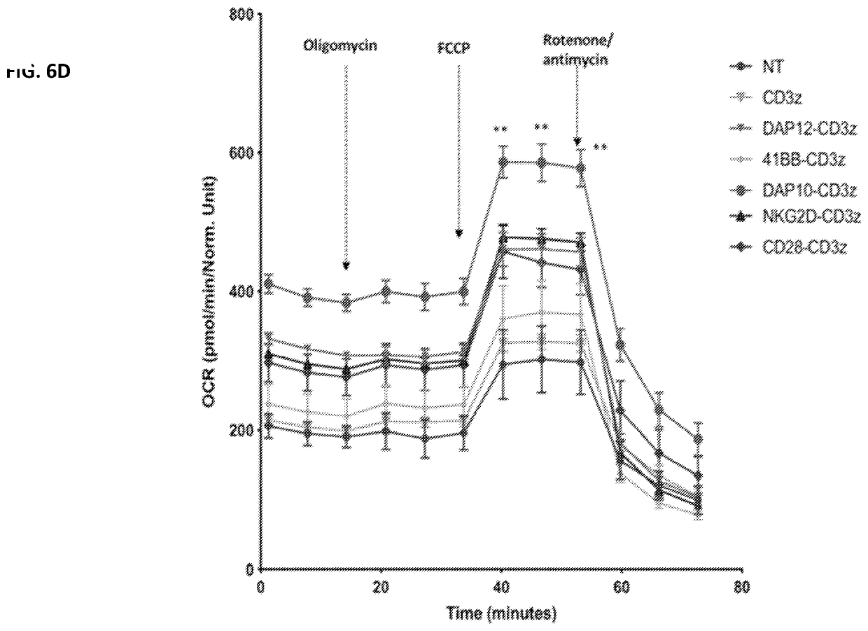

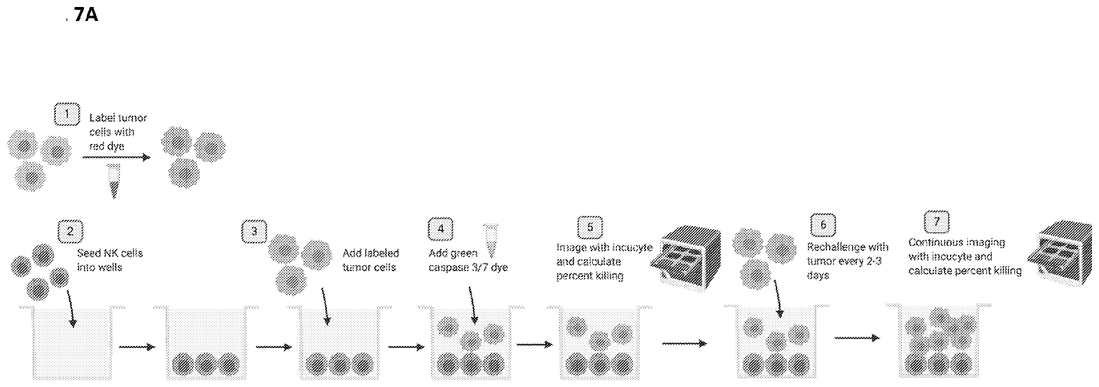

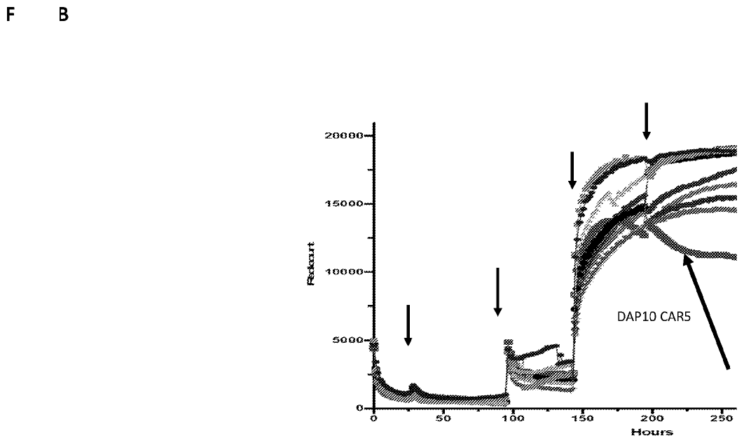

To gain insight into why CAR-NK cells with DAP10 signaling show less functional exhaustion in vivo compared to other CAR-NK cell designs, a series of experiments was performed to evaluate the polyfunctionality and the metabolism of CAR-NK cells with the various co-stimulatory domains. To evaluate cytokine secretion at the single cell level, the IsoPlexis single cell secretome assay was used, a well validated tool to evaluate polyfunctionality which correlates with immune cell potency. Following stimulation with a plate bound CD5 antigen for 4 hrs, CAR-NK cells with DAP10 signaling showed the highest polyfunctionality with a larger proportion of cells secreting 3, 4 or 5+ cytokines at the single cell level (FIG. 6A,6B). A higher proportion of these secreted cytokines in the DAP10 containing CD5 CAR-NK cells belonged to effector, stimulatory or chemoattractant categories (FIG. 6C). From a metabolic standpoint, CD5 CAR-NK cells with DAP10 signaling demonstrated a significantly higher oxygen consumption rate (OCR), a measure of oxidative phosphorylation and mitochondrial fitness, compared to the other constructs and to control NT NK cells (FIG. 6D).

CD5 CAR-NK Cells with DAP10 Co-Stimulatory Domain Display Memory Like Characteristics.

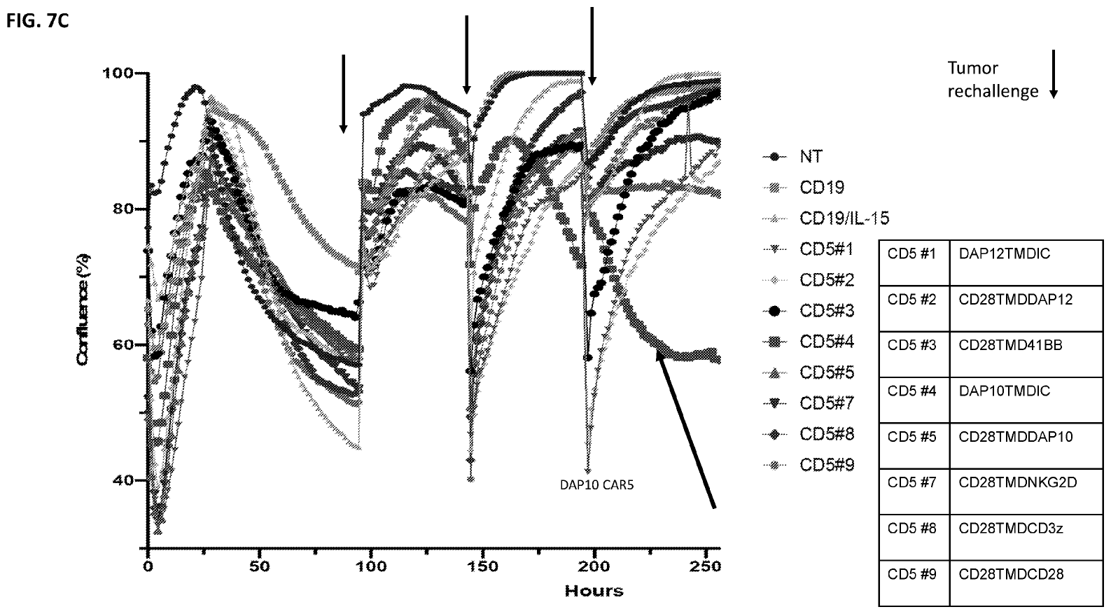

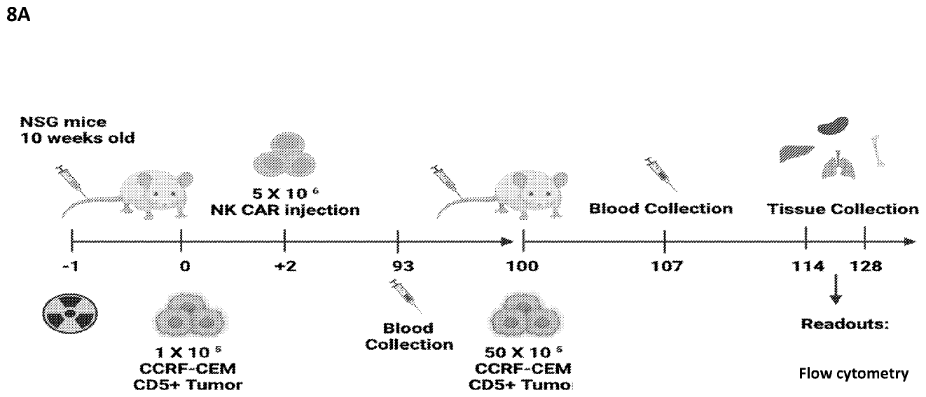

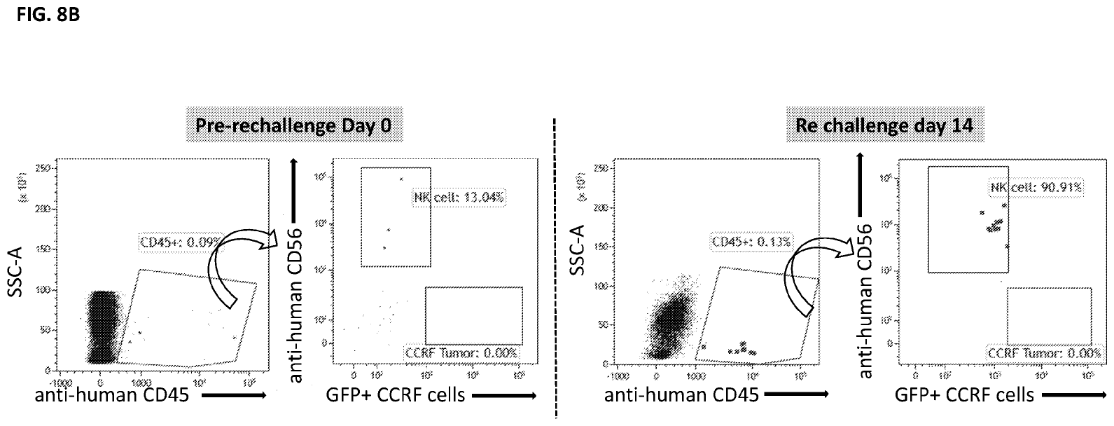

Next, it was examined if the favorable transcriptomic, proteomic and metabolic changes that accompany DAP10 co-stimulation in CD5 CAR-NK cells translate into memory like features which could explain the remarkable in vivo efficacy. Therefore, an in vivo setting was simulated where CAR-NK cells have to exhibit serial killing ability in order to adequately control the tumor and hence are prone to exhaustion. In a long-term Incucyte® cytotoxicity assay with multiple tumor rechallenges performed every 2-3 days (FIG. 7A), CD5 CAR-NK cells with DAP10 signaling exhibited superior cytotoxicity against CD5+CCRF-CEM compared to the other CAR-NK cell designs with persistent ability to kill following multiple tumor rechallenges, while NT NK cells, NK cells transduced with irrelevant target CD19 or CD19/IL-15 CAR and NK cells transduced with other CD5 CAR constructs lost the ability to kill over time (FIG. 7B,C). Similarly, in the in vivo model, the mice treated with DAP10 containing CD5 CAR-NK cells that survived long-term and seemed to be cured from disease were rechallenged with CD5+CCRF-CEM and developed no evidence of tumor development, which suggests that these CD5 CAR-NK cells are endowed with memory features and can eradicate tumor upon re-exposure (FIG. 7D-7F).

### Examples of Materials and Methods

### Cell Lines and Culture Media

CCRF-CEM (T-ALL cell line) was purchased from American Type Culture Collection (Manassa, VA, USA). CCRF-CEM cells were transduced with Firefly luciferase (CCRF-CEM-FFluc) for the in vivo experiments. For low CD5 model, shRNA was used to knockdown expression of CD5 in CCRF-CEM. K562 based feeder cells were retrovirally transduced to co-express 4-1BBL, CD48 and membrane bound IL-21 and are referred to as uAPCs. All cell lines were cultured in Roswell Park Memorial Institute (RPMI) medium supplemented with 10% Fetal Bovine Serum (FBS), 1% Penicillin-Streptomycin and 1% L-Glutamine. NK cells were cultured in Stem Cell Growth Medium (SCGM) supplemented with 10% Fetal Bovine Serum (FBS), 1% Penicillin-Streptomycin and 1% L-Glutamine.

### Cord Blood NK Cell Expansion

CB units for research were provided by the MD Anderson Cancer Center CB Bank. CBs was isolated by a density-gradient technique (Ficoll-Histopaque; Sigma, St Louis, MO, USA). CD56+NK cells, purified with an NK isolation kit (Miltenyi Biotec, Inc., San Diego, CA, USA), were stimulated with irradiated (100 Gy) uAPC (2:1 feeder cell:NK ratio) and recombinant human IL-2 (Proleukin, 200 U/ml; Chiron, Emeryville, CA, USA) in complete stem cell growth medium (CellGenix GmbH, Freiburg, Germany) on day 0. Activated NK cells were transduced with retroviral supernatants on day +6 in human fibronectin-coated plates (Clontech Laboratories, Inc., Mountain View, CA, USA). On day +7 and day +14, NK cells were stimulated again with irradiated uAPC and IL-2. On day +21, CAR-transduced NK cells were collected for use in the previously described experiments.

### Retrovirus Transfection and Transduction

The retroviral vectors encoding the various CD5 constructs were designed inhouse and the plasmids ordered from GeneArt™. CD19-CD28-CD3z and CD19-CD28-CD3z-IL-15 vectors have been previously described.⁶ Transient retroviral supernatants were produced as previously described.¹⁴ Activated NK cells were transduced with retroviral supernatants on day +6 in human fibronectin-coated plates (Clontech Laboratories, Inc., Mountain View, CA, USA). On day +8, CAR transduction efficiency was measured by flow cytometry and NK cells were stimulated again with irradiated uAPC and IL-2.

### Functional Assays

CAR19 or CAR19/IL-15 NK cells, or CD5 CAR-NK cells at 100×10e3 cells/well were co-cultured in the presence of Brefeldin A for 6 h in round bottom 96-well plates with CCRF-CEM cells or K562 targets (positive control) at an effector:target cell ratio (E:T) of 2:1. To measure degranulation, CD107a antibody (Brilliant Violet 785™ anti-human CD107a (LAMP-1) Antibody, Biolegend, San Diego, CA, USA) was added to the wells at the beginning of co-culture. After collection and surface staining of the cells for CD56, CD3 and CAR, cells were fixed and intracellular staining was done for TNFα (TNF alpha Monoclonal Antibody (MAb11), Alexa Fluor 700, eBioscience Inc., San Diego, CA, USA) and IFNγ (BD Horizon™ V450 Mouse Anti-Human IFNγ, BD Biosciences, San Jose, CA, USA). Cells were assessed by flow cytometry as previously described. 15

### Cytotoxicity Assays

### Chromium Release Assay

To assess cytotoxicity, ex-vivo expanded NT or CAR-transduced NK cells were co-cultured with ⁵¹Cr-labeled CCRF-CEM targets at multiple E:T ratios; cytotoxicity was measured by ⁵¹Cr release as previously described.¹⁵

### Incucyte Live Cell Imaging Assay

Ex vivo expanded NT and CAR-transduced NK cells were co-cultured in 96 well or 48 well plates at 1:1 E:T ratio with CCRF-CEM tumor targets transduced with a lentivirus to express a red dye (PE-Texas red). Each well contained 5×104 CCRF-CEM cells and 5×104 NK cells. Apoptosis was detected using the CellEvent™ Caspase-3/7 Green Detection Reagent (ThermoFisher). Wells containing tumor alone or NK cell alone groups served as negative controls. Frames were captured over time at 1-hour intervals from four separate 1.75×1.29 mm² regions per well with a 10× objective using IncuCyte S3 live-cell analysis system (Sartorius). Values from all four regions of each well were pooled and averaged across all three replicates. Results were expressed graphically as percent cytotoxicity by calculating the ratio of overlapping red and green signal (counts per image) divided by red signal (counts per image). For the rechallenge experiments, CCRF-CEM were added every 2-3 days to all wells (except control wells containing NK cell alone groups). The red count and confluence were plotted over time to follow tumor growth or elimination by NK cells.

### Mass Cytometry

### Antibody Conjugation

A panel comprising 38 metal-tagged antibodies was used for the in-depth characterization of NK cells. The list of antibodies with the corresponding metal tag isotopes is shown in Table. All unlabeled antibodies were purchased in carrier-free form and conjugated in-house with the corresponding metal tag using Maxpar X8 or MCP9 polymer per manufacturer's instructions (Fluidigm). All metal isotopes were acquired from Fluidigm except for indium (III) chloride (Sigma-Aldrich, St. Louis, MO). Antibody concentration was determined by measuring the amount of A280 protein using Nanodrop 2000 (Thermo Fisher Scientific, Waltham, MA). Conjugated antibodies were then diluted in PBS-based antibody stabilization solution or LowCross-Buffer (Candor Bioscience GmbH, Wangen, Germany) supplemented with 0.05% sodium azide (Sigma-Aldrich, St. Louis, MO) to a final concentration of 0.5 mg/ml. Serial titration experiments were performed to determine the concentration with the optimal signal to noise ratio for each antibody.

TABLE 1
Antibodies

| | | Clone | Vendor |
|---|---|---|---|
| 89Y | CD45 | HI30 | Fluidigm |
| 141Pr | LFA1 | m24 | Biolegend |
| 142Nd | ZAP70 | REA814 | Miltenyi |
| 143Nd | CD5 | UCHT2 | Fluidigm |
| 144Nd | Siglec 7 | NA | R&D |
| 145Nd | CD62L | DREG-56 | Biolegend |
| 146Nd | DAP12 | 134591 | Biolegend |
| 147Sm | NKG2C | REA205 | Miltenti |
| 148Nd | TRAIL | REA1113 | Miltenyi |
| 149Sm | CD25 | REA570 | Milteny Biotec |
| 150Nd | CD69 | REA824 | Milteny Biotec |
| 151Eu | CD2 | REA972 | Milteny Biotec |
| 152Sm | CD3z | 1D4 | BD Biosciences |
| 153Eu | PANKIR | 180704 | R&D |
| 154Sm | TIGIT | REA1004 | Milteny Biotec |
| 155Gd | CD27 | REA499 | Milteny Biotec |
| 156Gd | KLRG1 | REA261 | Milteny Biotec |
| 158Gd | OX40 | REA621 | Miltenyi Biotec |
| 159Tb | Perforin | REA1061 | Milteny Biotec |
| 160Gd | PD1 | PD1.3.1.3 | Milteny Biotec |
| 161Dy | T-BET | REA102 | Milteny Biotec |
| 162Dy | Eomes | WD1928 | Thermo Fisher |
| 163Dy | C-kit | REA787 | Milteny Biotec |
| 164Dy | CD95 | REA738 | Milteny Biotec |
| 165Ho | TIM3 | 344823 | R&D Systems |
| 166Er | NKG2D | REA797 | Miltenyi Biotec |
| 167Er | 2B4 | REA122 | Miltenyi Biotec |
| 168Er | KI67 | Custom Order | Miltenyi Biotec |
| 169Tm | NKG2A | REA110 | Milteny Biotec |
| 170Er | DNAM | REA1040 | Milteny Biotec |
| 171Yb | CD161 | REA631 | Miltenyi Biotec |
| 172Yb | CD38 | HIT2 | Fluidigm |
| 173Yb | CS1 | 162.1 | Biolegend |
| 174Yb | CD94 | REA113 | Milteny Biotec |
| 175Lu | LAG3 | REA351 | Milteny Biotec |
| 176Yb | ICOS | REA192 | Milteny Biotec |
| 209Bi | CD16 | 3G8 | Fluidigm |
| 115In | CD57 | REA769 | Milteny Biotec |
| Pt194 | CD3 | UCHT1 | Biolegend |
| 106Cd | CD56 | REA196 | Milteny Biotec |
| 110Cd | Granzyme B | REA226 | Milteny Biotec |
| 111Cd | Granzyme A | REA162 | Milteny Biotec |
| 112Cd | HLA DR | L243 | Biolegend |
| 113Cd | NKP30 | AF29-4D12 | Milteny Biotec |
| 114Cd | NKP46 | REA808 | Milteny Biotec |
| 116Cd | NKP44 | REA1163 | Milteny Biotec |
| Pt198 | LD | | |

### Sample Preparation and Acquisition:

NT or the various CD5 CAR transduced NK cells were harvested, washed twice with cell staining buffer (0.5% Bovine Serum Albumin, 0.02% Sodium Azide in PBS) at 4° C., 400 g for 5 min and incubated with 2.5 M Cisplatin-198Pt in the dark for 2 minutes. Then, the cells were washed once with FACS buffer (2% Fetal Bovine Serum in PBS) and once with cell staining buffer. The 10 μl of human Fc receptor blocking solution (Milteny Biotec, 130-059-901) for 10 minutes at room temperature. Cells were then stained with a freshly prepared antibody mix against cell surface markers diluted in 1% Bovine Serum Albumin in PBS for 40 minutes at room temperature on a shaker (130 rpm). Cells were washed twice with cell staining buffer and fixed using 1.8% paraformaldehyde in the dark at room temperature for 10 min. Cells were washed twice with cell staining buffer at 4° C., 800 g for 5 min and stored overnight in −80° C. in 1 ml of methanol. The next day, samples were washed twice with cell staining buffer, stained with a freshly prepared antibody mix against intracellular markers diluted in 1% Bovine Serum Albumin+0.2% Saponin in PBS. Cells were washed twice with cell staining buffer and incubated in 1 ml Intercalator Irridium (1 ul of 125 uM Ir in 1 ml PBS) for 20 minutes at room temperature. Cells were washed twice in cell staining buffer and resuspended in 1 ml of 0.1% Bovine Serum Albumin in Milli-Q® dH2O, filtered through a 35 μm nylon mesh (cell strainer cap tubes, BD, San Jose, CA) and counted. Before analysis, samples were suspended in Milli-Q® dH2O supplemented with EQ™ four element calibration beads at a concentration of 0.5×105/ml. Samples were acquired at 300 events/second on a Helios instrument (Fluidigm) using the Helios 6.5.358 acquisition software (Fluidigm).

### Data Analysis

Mass cytometry data were normalized based on EQ™ four element signal shift over time using Fluidigm normalization software 2. Initial data quality control was performed using Flowjo version 10.2. Calibration beads were gated out and singlets were chosen based on iridium 193 staining and event length. Dead cells were excluded by the Pt195 channel and further gating was performed to select CD45+ cells and then the NK cell population of interest (CD3-CD56+). A total of 320,000 cells were proportionally sampled from all samples to perform automated clustering. Data were analyzed using automated dimension reduction including (viSNE) in combination with FlowSOM for clustering¹⁶ for the deep phenotyping of immune cells as previously published.¹⁷

### Isoplexis Single Cell Secretome Assay

NT NK cells or the various CD5 CAR-NK cells were labeled with a fluorescent dye (Isoplexis Stain cell membrane 405) and either stimulated with plate bound CD5 antigen for 4 hrs or co-cultured with CCRF-CEM cells for 4 hrs then purified using positive selection with CD56+ microbeads. From the purified population, 3×10⁴ NK cells were loaded onto individual Isocode chips according to the manufacturer's protocol (Isoplexis, Branford, CT, USA) and subjected to single cell 32-plex cytokine secretome profiling using a fully validated panel of cytokines on the IsoPlexis single-cell platform. As previously described,^{18,19} the IsoSpeak software was used to quantify the number of polyfunctional NK cells, measure the percentage of cells in each sample secreting multiple cytokines (2+, 3+, 4+ or 5+), assign the category of cytokines being secreted (effector, stimulatory, regulatory or chemoattractive) and measure the polyfunctionality strength index (PSI) defined as the percentage of polyfunctional cells, multiplied by mean fluorescence intensity (MFI) of the proteins secreted by the NK cells.

### Seahorse Assays

NT-NK cells or the various CD5 CAR-transduced NK cells were assayed using the Seahorse XF Glycolysis Stress and Mito Stress Test Kits (Agilent). The extracellular acidification rate (ECAR) and oxygen consumption rate (OCR) were measured using the Agilent Seahorse XFe96 Analyzer (Agilent) per the manufacturer's instructions.

### Xenogeneic Mouse Models

To assess the antitumor effect of CD5 CAR-transduced CB-NK cells in vivo, a NOD/SCID IL-2Rγnull (NSG) xenograft model was used that was engrafted with a CCRF-CEM cell line. Mouse experiments were performed in accordance with NIH recommendations under protocols approved by the Institutional Animal Care and Use Committee. NSG mice (10-12 weeks old; Jackson Laboratories, Bar Harbor, ME, USA) were irradiated with 300 cGy at day-1 and inoculated intravenously with firefly luciferase-labeled CCRF-CEM cells (1×10e5) on day 0. Where indicated, fresh expanded NT or CD5 CAR-transduced CB-NK cells were injected through the tail vein on day 2. Mice were subjected to weekly bioluminescence imaging (Xenogen-IVIS 200 Imaging system; Caliper, Waltham, MA, USA). Signal quantitation in photons/second was performed by determining the photon flux rate within standardized regions of interest using Living Image software (Caliper).²⁰ Trafficking, persistence and expansion of NK cells were measured by flow cytometry. Mice were also monitored for survival and for toxicity by recording weights and scoring.

### Antibodies Used to Examine the In Vivo Trafficking, Persistence and Expansion of NK Cells

The following antibodies were used for flow cytometry staining: APC/Cy7 anti-human CD3 Antibody (clone HIT3a, Biolegend), Brilliant Violet 605TM anti-human CD56 (NCAM) Antibody (Clone HCD56, Biolegend), anti-CAR antibody Goat F(ab′)2 anti-Human IgG (H+L)-Alexa Fluor 647 (Jackson), PE Mouse Anti-Human CD7 Clone M-T701 (BD biosciences), BV711 Mouse Anti-Human CD5 Clone UCHT2 (BD biosciences), BV650 Mouse Anti-Human CD16 Clone 3G8 (BD biosciences), PerCP anti-human CD45 Antibody Clone HI30 (Biolegend), Live/dead aqua dead cell stain (ThermoFisher).

### Statistics

Two-way Anova test was used to compare quantitative differences (mean±sd) between groups; P-values were two-sided and P<0.05 was considered significant. For all bioluminescence experiments, intensity signals were summarized as means±sd at baseline and at multiple subsequent time points for each group of mice.²⁰ Probabilities of survival were calculated using the Kaplan-Meier method. Statistical tests indicated were performed using Prism software (GraphPad version 7.0c).

### Significance of Certain Embodiments

The effects of various co-stimulatory domains on CAR-T cell function and fitness have been well studied. In the CAR-NK cell field, few studies investigating the role of different co-stimulatory molecules have been reported, but no studies have systematically looked at the impact of the choice of co-stimulation on the proliferation, transcriptomic profile, phenotype, polyfunctionality, metabolism, cytotoxicity and memory formation. The study delves into these specific questions and shows that DAP10, an NK-centric co-stimulatory domain, leads to favorable phenotypic changes and synergistic signaling culminating in optimal global anti-tumor activity and fitness of CD5 CAR-NK cells and features of memory formation.

In CAR-T cells, the impact of 4-1BB versus CD28 co-stimulation on the signaling and metabolism of T cells has been thoroughly investigated and correlated to clinical outcomes and data regarding persistence and exhaustion of the cellular therapy product.²¹ DAP10 has been previously shown to enhance CAR-T cell and NKG2D based CAR-NK cell function^{22,23}, but no mechanistic insight has been offered to explain its advantage over other co-stimulatory molecules. Furthermore, the signaling pathways downstream of DAP10 co-stimulation have not been dissected in the setting of CAR-T or CAR-NK cells.

### REFERENCES

The following references, to the extent that they provide exemplary procedural or other details supplementary to those set forth herein, are specifically incorporated herein by reference.
1. Tsuyama N, Ennishi D, Yokoyama M, et al. Clinical and prognostic significance of aberrant T-cell marker expression in 225 cases of de novo diffuse large B-cell lymphoma and 276 cases of other B-cell lymphomas. Oncotarget. 2017; 8(20):33487-33500.
2 Bashford-Rogers R J, Palser A L, Hodkinson C, et al. Dynamic variation of CD5 surface expression levels within individual chronic lymphocytic leukemia clones. Exp Hematol. 2017; 46:31-37 e10.
3. Scherer L D, Brenner M K, Mamonkin M. Chimeric Antigen Receptors for T-Cell Malignancies. Front Oncol. 2019; 9:126.
4. Mamonkin M, Rouce R H, Tashiro H, Brenner M K. A T-cell-directed chimeric antigen receptor for the selective treatment of T-cell malignancies. Blood. 2015; 126(8): 983-992.
5. Hill L R R, Smith T et al. Safety and Anti-Tumor Activity of CD5 CAR T-Cells in Patients with Relapsed/Refractory T-Cell Malignancies. Blood. 2019; 134 (Supplement_1): 199. https://doi.org/10.1182/blood-2019-129559.
6. Liu E, Tong Y, Dotti G, et al. Cord blood N K cells engineered to express IL-15 and a CD19-targeted CAR show long-term persistence and potent antitumor activity. Leukemia. 2018; 32(2):520-531.
7. Liu E, Marin D, Banerjee P, et al. Use of CAR-Transduced Natural Killer Cells in CD19-Positive Lymphoid Tumors. N Engl J Med. 2020; 382(6):545-553.
8 Kawalekar O U, O'Connor R S, Fraietta J A, et al. Distinct Signaling of Coreceptors Regulates Specific Metabolism Pathways and Impacts Memory Development in CAR T Cells. Immunity. 2016; 44(2):380-390.
9 Guedan S, Posey A D, Jr., Shaw C, et al. Enhancing CAR T cell persistence through ICOS and 4-1B B costimulation. JCI Insight. 2018; 3(1).
10. Zhang H, Snyder K M, Suhoski M M, et al. 4-1B B is superior to CD28 costimulation for generating CD8+ cytotoxic lymphocytes for adoptive immunotherapy. J Immunol. 2007; 179(7):4910-4918.
11. Guerra N, Tan Y X, Joncker N T, et al. NKG2D-deficient mice are defective in tumor surveillance in models of spontaneous malignancy. Immunity. 2008; 28(4):571-580.
12. Liu E, Ang S O T, Kerbauy L, et al. GMP-Compliant Universal Antigen Presenting Cells (uAPC) Promote the Metabolic Fitness and Antitumor Activity of Armored Cord Blood CAR-N K Cells. Front Immunol. 2021; 12:626098.
13. Lanier L L. Up on the tightrope: natural killer cell activation and inhibition. Nat Immunol. 2008; 9(5):495-502.
14. Vera J, Savoldo B, Vigouroux S, et al. T lymphocytes redirected against the kappa light chain of human immunoglobulin efficiently kill mature B lymphocyte-derived malignant cells. Blood. 2006; 108(12):3890-3897.
15. Rouce R H, Shaim H, Sekine T, et al. The TGF-beta/SMAD pathway is an important mechanism for N K cell immune evasion in childhood B-acute lymphoblastic leukemia. Leukemia. 2016; 30(4):800-811.
16. Van Gassen S, Callebaut B, Van Helden M J, et al. FlowSOM: Using self-organizing maps for visualization and interpretation of cytometry data. Cytometry A. 2015; 87(7):636-645.
17. Kordasti S, Costantini B, Seidl T, et al. Deep-phenotyping of Tregs identifies an immune signature for idiopathic aplastic anemia and predicts response to treatment. Blood. 2016.
18. Rossi J, Paczkowski P, Shen Y W, et al. Preinfusion polyfunctional anti-CD19 chimeric antigen receptor T cells are associated with clinical outcomes in NHL. Blood. 2018; 132(8):804-814.
19. Xue Q, Bettini E, Paczkowski P, et al. Single-cell multiplexed cytokine profiling of CD19 CAR-T cells reveals a diverse landscape of polyfunctional antigen-specific response. J Immunother Cancer. 2017; 5(1):85.
20. Shah N, Martin-Antonio B, Yang H, et al. Antigen presenting cell-mediated expansion of human umbilical cord blood yields log-scale expansion of natural killer cells with anti-myeloma activity. PLOS One. 2013; 8(10):e76781.
21. Kawalekar O U, R S O C, Fraietta J A, et al. Distinct Signaling of Coreceptors Regulates Specific Metabolism Pathways and Impacts Memory Development in CAR T Cells. Immunity. 2016; 44(3):712.
22. Chang Y H, Connolly J, Shimasaki N, Mimura K, Kono K, Campana D. A chimeric receptor with NKG2D specificity enhances natural killer cell activation and killing of tumor cells. Cancer Res. 2013; 73(6): 1777-1786.
23. Zhao R, Cheng L, Jiang Z, et al. DNAX-activating protein 10 co-stimulation enhances the anti-tumor efficacy of chimeric antigen receptor T cells. Oncoimmunology. 2019; 8(1):e1509173.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An expression construct comprising sequence that encodes a CD5-specific engineered receptor, wherein said receptor comprises one of the following:
(a) CD28 transmembrane domain (TM) and CD28 intracytoplasmic domain (ICD);
(b) CD28 TM, CD28 ICD, and CD3zeta signaling domain;
(c) CD28 TM and DAP12 ICD;
(d) CD28 TM, DAP12 ICD, and CD3zeta signaling domain;
(e) DAP12 TM and DAP12 ICD;
(f) DAP12 TM, DAP12 ICD, and CD3zeta signaling domain;
(g) CD28 TM and 4-1BB ICD; (h) CD28 TM, 4-1BB ICD and CD3zeta signaling domain;
(h) CD28 TM, CD28 ICD, and 4-1BB ICD;
(i) CD28 TM, CD28 ICD, 4-1BB ICD, and CD3zeta signaling domain;
(j) CD28 TM and DAP10 ICD;
(k) CD28TM, DAP10 ICD, and CD3zeta signaling domain;
(l) DAP10 TM and DAP10 ICD;
(m) DAP10 TM, DAP10 ICD, and CD3zeta signaling domain;
(n) NKG2D ICD;
(o) NKG2D TM;
(p) NKG2D TM and NKG2D ICD;
(q) NKG2D TM, NKG2D ICD, and CD3zeta signaling domain;
(r) CD28 TM and NKG2D ICD;
(s) NKG2D ICD, and CD3zeta signaling domain;
(t) CD28 TM, NKG2D ICD, and CD3zeta signaling domain;
(u) 4-1BB TM and 4-1BB ICD;
(v) CD28TM and CD3zeta in the absence of an ICD that is a costimulatory domain;
(w) CD8 TM and CD28 intracytoplasmic domain (ICD);
(x) CD8 TM, CD28 ICD, and CD3zeta signaling domain;
(y) CD8 TM and DAP12 ICD;
(z) CD8 TM, DAP12 ICD, and CD3zeta signaling domain;
(aa) CD8 TM and DAP12 ICD;
(bb) CD8 TM, DAP12 ICD, and CD3zeta signaling domain;
(cc) CD8 TM and 4-1BB ICD; (ee) CD8 TM, 4-1BB ICD and CD3zeta signaling domain;
(dd) CD8 TM, CD28 ICD, and 4-1BB ICD;
(ee) CD8 TM, CD28 ICD, 4-1BB ICD, and CD3zeta signaling domain;
(ff) CD8 TM and DAP10 ICD;
(gg) CD8 TM, DAP10 ICD, and CD3zeta signaling domain;
(hh) CD8 TM and DAP10 ICD;
(ii) CD8 ICD;
(jj) CD8 TM;
(kk) CD8 TM and NKG2D ICD;
(ll) CD8 TM, NKG2D ICD, and CD3zeta signaling domain;
(mm) CD8TM and CD3zeta in the absence of an ICD that is a costimulatory domain
(nn) CD27 TM and CD28 intracytoplasmic domain (ICD);
(oo) CD27 TM, CD28 ICD, and CD3zeta signaling domain;
(pp) CD27 TM and DAP12 ICD;
(qq) CD27 TM, DAP12 ICD, and CD3zeta signaling domain;
(rr) CD27 TM and DAP12 ICD;
(ss) CD27 TM, DAP12 ICD, and CD3zeta signaling domain;
(tt) CD27 TM and 4-1BB ICD;
(uu) CD27 TM, 4-1BB ICD and CD3zeta signaling domain;
(vv) CD27 TM, CD28 ICD, and 4-1BB ICD;
(ww) CD27 TM, CD28 ICD, 4-1BB ICD, and CD3zeta signaling domain;
(xx) CD27 TM and DAP10 ICD;
(yy) CD27 TM, DAP10 ICD, and CD3zeta signaling domain;
(zz) CD27 TM and DAP10 ICD;
(aaa) CD27 TM;
(bbb) CD27 TM and NKG2D ICD;
(ccc) CD27 TM, NKG2D ICD, and CD3zeta signaling domain; or
(ddd) CD27TM and CD3zeta in the absence of an ICD that is a costimulatory domain;

2. The expression construct of claim 1, wherein the CD5-specific engineered receptor is a chimeric antigen receptor (CAR) or a T cell receptor.

3. The expression construct, wherein the CD5-specific engineered receptor is a CAR.

4. The expression construct of claim 3, wherein the CAR comprises a CD-targeting extracellular domain that is an scFv or a ligand for CD5.

5. The expression construct of claim 4, wherein the CD5-specific CAR comprises a scFv having a heavy chain and a light chain, and wherein the heavy chain in the sequence that encodes the CAR is upstream of the light chain in a 5′ to 3′ direction.

6. The expression construct of claim 4, wherein the CD5-specific CAR comprises a scFv having a heavy chain and a light chain, and wherein the heavy chain in the sequence that encodes the CAR is downstream of the light chain in a 5′ to 3′ direction.

7. The expression construct of any one of claims 4-6, wherein the CD5-specific CAR comprises a codon optimized scFv.

8. The expression construct of any one of claims 4-6, wherein the CD5-specific CAR comprises a humanized scFv.

9. The expression construct of any one of claim 4-6 or 8, wherein the scFv is encoded by SEQ ID NO:1.

10. The expression construct of any one of claim 4-6 or 8, wherein the scFv comprises SEQ ID NO:2.

11. The expression construct of any one of claims 4-6, wherein the scFv is encoded by SEQ ID NO:3.

12. The expression construct of any one of claims 4-6, wherein the scFv comprises SEQ ID NO:4.

13. The expression construct of any one of claims 3-12, wherein the CD5-specific CAR comprises a signaling peptide.

14. The expression construct of claim 13, wherein the signaling peptide is from CD8alpha, Ig heavy chain, granulocyte-macrophage colony-stimulating factor receptor, CD3 signaling peptide, CD4 signaling peptide, or a signal peptide derived from one or more other surface receptors.

15. The expression construct of any one of claims 1-14, wherein the TM is encoded by SEQ ID NO:5, SEQ ID NO:7, SEQ ID NO:9, SEQ:11 or SEQ ID NO: 13.

16. The expression construct of any one of claims 1-14, wherein the TM comprises SEQ ID NO:6, SEQ ID NO:8, SEQ ID NO: 10, SEQ: 12 or SEQ ID NO: 14.

17. The expression construct of any one of claims 1-16, wherein the ICD is encoded by SEQ ID NO:15, SEQ ID NO:17, SEQ ID NO: 19, or SEQ ID NO:21.

18. The expression construct of any one of claims 1-16, wherein the ICD comprises SEQ ID NO:16, SEQ ID NO: 18, SEQ ID NO:20, or SEQ ID NO:22.

19. The expression construct of any one of claims 1-18, wherein the receptor further comprises an ICD from CD27, OX-40 (CD134), CD40L, 2B4, DNAM, CS1, CD48, NKp30, NKp44, NKp46, NKp80, ICOS, or a combination thereof.

20. The expression construct of any one of claims 1-19, wherein the CD5-specific engineered receptor is a CD5-specific CAR that comprises a hinge between a CD5-specific scFv and the transmembrane domain.

21. The expression construct of claim 20, wherein the hinge is an IgG hinge, a CD28 hinge, a CD8-alpha hinge, the hinge comprises an artificial spacer comprised of Gly3, or the hinge comprises CH1, CH2, and/or CH3 domains of IgGs.

22. The expression construct of claim 21, wherein the IgG hinge is IgG1 hinge, IgG2 hinge, IgG3 hinge, or IgG4 hinge.

23. The expression construct of claim 21, wherein the hinge is encoded by SEQ ID NO:23 or 25.

24. The expression construct of claim 21, wherein the hinge comprises SEQ ID NO:24 or SEQ ID NO:26.

25. The expression construct of any one of claims 1-24, wherein the cytokine is IL-15, IL-12, IL-2, IL-18, IL-21, IL-7, IL-23, or a combination thereof.

26. The expression construct of any one of claims 1-25, wherein the CD5-specific engineered receptor is a CAR encoded by SEQ ID NO:33, SEQ ID NO:35, SEQ ID NO:37, SEQ ID NO:39, SEQ ID NO:41, SEQ ID NO:43, SEQ ID NO:45, or SEQ ID NO:47.

27. The expression construct of any one of claims 1-25, wherein the CD5-specific engineered receptor is a CAR comprising SEQ ID NO:34, SEQ ID NO:36, SEQ ID NO:38, SEQ ID NO:40, SEQ ID NO:42, SEQ ID NO:44, SEQ ID NO:46, or SEQ ID NO:48.

28. The expression construct of any one of claims 1-27, wherein the expression construct encodes a polypeptide of interest other than the CD5-specific engineered receptor.

29. The expression construct of claim 28, wherein the polypeptide of interest and the CD-specific engineered receptor are separated on the construct by a 2A element or an IRES element.

30. The expression construct of claim 29, wherein the 2A element is E2A, T2A, or P2A.

31. The expression construct of claim 30, wherein the E2A element is encoded by SEQ ID NO:31.

32. The expression construct of claim 30, wherein the E2A element comprises SEQ ID NO:32.

33. The expression construct of any one of claims 28-32, wherein the polypeptide of interest is a therapeutic protein or a protein that enhances cell activity, expansion, and/or persistence of cells comprising the protein.

34. The expression construct of any one of claims 28-33, wherein the polypeptide of interest is a suicide gene; cytokine; human or viral protein that enhances proliferation, expansion and/or metabolic fitness of cells comprising the protein; or a combination thereof.

35. The expression construct of claim 34, wherein the suicide gene is a nonsecretable mutant TNF-alpha, inducible caspase 9, HSV-thymidine kinase, CD19, CD20, CD52, or EGFRv3.

36. The expression construct of claim 34 or 35, wherein the cytokine is IL-15, IL-2, IL-12, IL-18, IL-21, IL-23, IL-7, or a combination thereof.

37. The expression construct of claim 36, wherein the IL-15 is encoded by SEQ ID NO:29.

38. The expression construct of claim 36, wherein the IL-15 comprises SEQ ID NO:30.

39. A vector, comprising the expression construct of any one of claims 1-38.

40. The vector of claim 39, wherein the vector is a viral vector or a non-viral vector.

41. The vector of claim 40, wherein the viral vector is an adenoviral vector, adeno-associated viral vector, lentiviral vector, or retroviral vector.

42. The vector of claim 40, wherein the non-viral vector is a plasmid, liposome, nanoparticle, lipid, carbohydrate, or combination thereof.

43. An immune cell, comprising the expression construct of any one of claims 1-38 or the vector of any one of claims 39-42.

44. The immune cell of claim 43, wherein the immune cell is a natural killer (NK) cell, T cell, gamma delta T cells, invariant NKT (iNKT) cell, B cell, macrophage, MSCs, or dendritic cell.

45. The immune cell of claim 43 or 44, wherein the immune cell is an NK cell.

46. The immune cell of claim 44 or 45, wherein the NK cell is derived from cord blood, peripheral blood, induced pluripotent stem cells, human embryonic stem cells, bone marrow, or from a cell line.

47. The immune cell of claim 46, wherein the NK cell line is NK-92 cell line or another NK cell line derived from a tumor or from a healthy NK cell or a progenitor cell.

48. The immune cell of any one of claims 44-47, wherein the NK cell is derived from a cord blood mononuclear cell.

49. The immune cell of any one of claims 44-48, wherein the NK cell is a CD56+ NK cell.

50. The immune cell of any one of claims 43-49, wherein the NK cells express one or more exogenously provided cytokines.

51. The immune cell of claim 50, wherein the cytokine is IL-15, IL-2, IL-12, IL-18, IL-21, IL-7, IL-23, or a combination thereof.

52. The immune cell of any one of claims 43-51, wherein expression of one or more endogenous genes in the immune cell has been modified.

53. The immune cell of claim 52, wherein the expression has been partially or fully reduced in expression.

54. The immune cell of claim 52 or 53, wherein expression of the one or more gene has been modified using CRISPR or TALEN.

55. The immune cell of any one of claims 52-54, wherein the gene is selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, CD7, CTLA-4, TDAG8, CD38, CREM, and a combination thereof.

56. A population of immune cells of any one of claims 43-55, said cells present in a suitable medium.

57. The population of claim 56, wherein said population is housed in a storage facility.

58. The population of claim 56 or 57, wherein said population is frozen.

59. The population of any one of claims 56-58, wherein said medium comprises a cryoprotectant with one or more cytokines or a cryoprotectant without one or more cytokines.

60. The population of any one of claims 56-59, wherein the immune cells are NK cells.

61. The population of any one of claims 56-60, wherein the immune cells are NK cells that comprise an expression construct that encodes a CAR and a cytokine.

62. The population of any one of claims 56-61, wherein the immune cells are NK cells that comprise an expression construct that encodes a CAR and IL-15.

63. The population of claim 61 or 62, wherein the CAR and IL-15 are encoded by SEQ ID NO:33, SEQ ID NO:35, SEQ ID NO:37, SEQ ID NO:39, SEQ ID NO:41, SEQ ID NO:43, SEQ ID NO:45, or SEQ ID NO:47.

64. The population of claim 61 or 62, wherein the CAR and IL-15 are comprised in SEQ ID NO:34, SEQ ID NO:36, SEQ ID NO:38, SEQ ID NO:40, SEQ ID NO:42, SEQ ID NO:44, SEQ ID NO:46, or SEQ ID NO:48.

65. A method of killing CD5-positive cells in an individual, comprising the step of administering to the individual an effective amount of cells harboring the expression construct of any one of claims 1-38.

66. The method of claim 65, wherein the cells are immune cells.

67. The method of claim 66, wherein the immune cells are NK cells, T cells, gamma delta T cells, invariant NKT (iNKT) cells, B cells, macrophages, dendritic cells, or a mixture thereof.

68. The method of claim 67, wherein the NK cells are derived from cord blood, peripheral blood, induced pluripotent stem cells, human embryonic stem cells, bone marrow, from a cell line, or a mixture thereof.

69. The method of any one of claims 67-68, wherein the NK cells are derived from cord blood mononuclear cells.

70. The method of any one of claims 34-37, wherein the individual has cancer.

71. The method of claim 70, wherein the cancer is of a solid tumor.

72. The method of claim 70, wherein the cancer is a hematological cancer.

73. The method of any one of claims 65-72, wherein the cells are allogeneic with respect to the individual.

74. The method of any one of claims 65-72, wherein the cells are autologous with respect to the individual.

75. The method of any one of claims 65-74, wherein the individual is a human.

76. The method of any one of claims 65-75, wherein the cells are administered to the individual once or more than once.

77. The method of claim 76, wherein the duration of time between administrations of the cells to the individual is 1-24 hours, 1-7 days, 1-4 weeks, 1-12 months, or one or more years.

78. The method of any one of claims 65-77, further comprising the step of providing to the individual an effective amount of an additional therapy.

79. The method of claim 78, wherein the additional therapy comprises surgery, radiation, gene therapy, immunotherapy, or hormone therapy.

80. The method of claim 78 or 79, wherein the additional therapy comprises one or more antibodies.

81. The method of any one of claims 65-80, wherein the cells are administered to the individual by injection, intravenously, intraarterially, intraperitoneally, intrapleurally, intratracheally, intratumorally, intramuscularly, endoscopically, intralesionally, intracranially, intrathecally, percutaneously, subcutaneously, regionally, by perfusion, in a tumor microenvironment, or a combination thereof.

82. The method of any one of claims 65-81, further comprising the step of identifying CD5-positive cancer in the individual.

83. The method of any one of claims 65-82, further comprising the step of producing the cells harboring the expression construct.

84. A method of preventing cancer or preventing metastasis of cancer in an individual, comprising the step of administering to the individual a therapeutically effective amount of cells harboring the expression construct of any one of claims 1-38.

85. The method of claim 84, wherein the individual is at risk for cancer or has previously had cancer.

86. The method of claim 84 or 85, wherein the administering step occurs once.

87. The method of claim 84 or 85, wherein the administering step occurs more than once.


SEQUENCE LISTING (55 sequences)

SEQ ID NO: 1 - DNA, 792 bp, Artificial Sequence
  Description of Artificial Sequence Synthetic polynucleotide
atggagttcggtctcagttggctgtttttggttgctatcttgaagggcgtccaatgcagccgggacatccagatgacccagtctccctctagcatgtcagcgagtcttggtgatcgagtgacgattacctgcagagcctctcaagatataaacagctatctttcatggttccaacagaagccggggaagtccccaaaaactctcatatacagggcgaatcgactcgtagacggtgtgccttcaaggttttccgggagtggtagtggcacagattacacacttacaatctcttcattgcagtatgaggatttcgggatctactactgtcaacagtacgacgaatccccatggacgtttgggggcgggaccaaacttgagataaaagggagcacatctggaagtggtaaacctgggtcaggggagggttccacaaaaggacaaattcaacttgtccaaagcggtcctggtcttaagaagcctggagggtctgtcaggataagttgtgcggcatccggctacaccttcaccaactatgggatgaactgggtgaaacaagcgcctgggaaaggtcttcgatggatgggctggattaatacccacactggagagcccacttacgctgatgatttcaaaggacgatttaccttctccttggatacttccaagagtaccgcgtacttgcaaatcaacagtctccgggctgaagacacggccacatacttctgtacgcggagagggtatgactggtattttgatgtgtggggtcagggaacaaccgtgactgtttcaagc

SEQ ID NO: 2 - protein, 264 aa, Artificial Sequence
  Description of Artificial Sequence Synthetic polypeptide
MEFGLSWLFLVAILKGVQCSRDIQMTQSPSSMSASLGDRVTITCRASQDINSYLSWFQQKPGKSPKTLIYRANRLVDGVPSRFSGSGSGTDYTLTISSLQYEDFGIYYCQQYDESPWTFGGGTKLEIKGSTSGSGKPGSGEGSTKGQIQLVQSGPGLKKPGGSVRISCAASGYTFTNYGMNWVKQAPGKGLRWMGWINTHTGEPTYADDFKGRFTFSLDTSKSTAYLQINSLRAEDTATYFCTRRGYDWYFDVWGQGTTVTVSS

SEQ ID NO: 3 - DNA, 792 bp, Artificial Sequence
  Description of Artificial Sequence Synthetic polynucleotide
atggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagc

SEQ ID NO: 4 - protein, 264 aa, Artificial Sequence
  Description of Artificial Sequence Synthetic polypeptide
MEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSS

SEQ ID NO: 5 - DNA, 81 bp, Homo sapiens
atcatctccttctttcttgcgctgacgtcgactgcgttgctcttcctgctgttcttcctcacgctccgtttctctgttgtt

SEQ ID NO: 6 - protein, 27 aa, Homo sapiens
IISFFLALTSTALLFLLFFLTLRFSVV

SEQ ID NO: 7 - DNA, 81 bp, Homo sapiens
ttttgggtgctggtggtggttggtggagtcctggcttgctatagcttgctagtaacagtggcctttattattttctgggtg

SEQ ID NO: 8 - protein, 27 aa, Homo sapiens
FWVLVVVGGVLACYSLLVTVAFIIFWV

SEQ ID NO: 9 - DNA, 63 bp, Homo sapiens
ctcctggcaggcctcgtggctgctgatgcggtggcatcgctgctcatcgtgggggcggtgttc

SEQ ID NO: 10 - protein, 21 aa, Homo sapiens
LLAGLVAADAVASLLIVGAVF

SEQ ID NO: 11 - DNA, 63 bp, Homo sapiens
ggcgtgctggcagggatcgtgatgggagacctggtgctgacagtgctcattgccctggccgtg

SEQ ID NO: 12 - protein, 21 aa, Homo sapiens
GVLAGIVMGDLVLTVLIALAV

SEQ ID NO: 13 - DNA, 63 bp, Homo sapiens
gcggtgatgattatttttcgcattggcatggcggtggcgattttttgctgctttttttttccg

SEQ ID NO: 14 - protein, 21 aa, Homo sapiens
AVMIIFRIGMAVAIFCCFFFP

SEQ ID NO: 15 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - protein, 42 aa, Homo sapiens
KRGRKKLLYIFKQPFMRPVQTTQEEDGCSCRFPEEEEGGCEL

SEQ ID NO: 17 - DNA, 72 bp, Homo sapiens
ctttgcgcacgcccacgccgcagccccgcccaagaagatggcaaagtctacatcaacatgccaggcaggggc

SEQ ID NO: 18 - protein, 24 aa, Homo sapiens
LCARPRRSPAQEDGKVYINMPGRG

SEQ ID NO: 19 - DNA, 156 bp, Homo sapiens
tacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 20 - protein, 52 aa, Homo sapiens
YFLGRLVPRGRGAAEAATRKQRITETESPYQELQGQRSDVYSDLNTQRPYYK

SEQ ID NO: 21 - DNA, 153 bp, Homo sapiens
agcgcgaacgaacgctgcaaaagcaaagtggtgccgtgccgccagaaacagtggcgcaccagctttgatagcaaaaaactggatctgaactataaccattttgaaagcatggaatggagccatcgcagccgccgcggccgcatttggggcatg

SEQ ID NO: 22 - protein, 51 aa, Homo sapiens
SANERCKSKVVPCRQKQWRTSFDSKKLDLNYNHFESMEWSHRSRRGRIWGM

SEQ ID NO: 23 - DNA, 742 bp, Artificial Sequence
  Description of Artificial Sequence Synthetic polynucleotide
gtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaatt

SEQ ID NO: 24 - protein, 246 aa, Artificial Sequence
  Description of Artificial Sequence Synthetic polypeptide
TVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 25 - DNA, 117 bp, Homo sapiens
attgaagttatgtatcctcctccttacctagacaatgagaagagcaatggaaccattatccatgtgaaagggaaacacctttgtccaagtcccctatttcccggaccttctaagccc

SEQ ID NO: 26 - protein, 39 aa, Homo sapiens
IEVMYPPPYLDNEKSNGTIIHVKGKHLCPSPLFPGPSKP

SEQ ID NO: 27 - DNA, 342 bp, Homo sapiens
cgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggac

SEQ ID NO: 28 - protein, 113 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 29 - DNA, 490 bp, Homo sapiens
gcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctgacaatt

SEQ ID NO: 30 - protein, 160 aa, Homo sapiens
ISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 31 - DNA, 60 bp, Equine rhinitis A virus
cagtgtactaattatgctctcttgaaattggctggagatgttgagagcaatcccgggccc

SEQ ID NO: 32 - protein, 20 aa, Equine rhinitis A virus
QCTNYALLKLAGDVESNPGP

SEQ ID NO: 33 - DNA, 2523 bp, Artificial Sequence
  Description of Artificial Sequence Synthetic polynucleotide
atgctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttggttttgggtgctggtggtggttggtggagtcctggcttgctatagcttgctagtaacagtggcctttattattttctgggtgacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 34 - protein, 840 aa, Artificial Sequence
  Description of Artificial Sequence Synthetic polypeptide
MLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWFWVLVVVGGVLACYSLLVTVAFIIFWVTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 35 - DNA, 2577 bp, Artificial Sequence
  Description of Artificial Sequence Synthetic polynucleotide
atgctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttggctcctggcaggcctcgtggctgctgatgcggtggcatcgctgctcatcgtgggggcggtgttcctttgcgcacgcccacgccgcagccccgcccaagaagatggcaaagtctacatcaacatgccaggcaggggcacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 36 - protein, 858 aa, Artificial Sequence
  Description of Artificial Sequence Synthetic polypeptide
MLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWLLAGLVAADAVASLLIVGAVFLCARPRRSPAQEDGKVYINMPGRGTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 37 - DNA, 2649 bp, Artificial Sequence
  Description of Artificial Sequence Synthetic polynucleotide
atgctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttggttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 38 - protein, 882 aa, Artificial Sequence
  Description of Artificial Sequence Synthetic polypeptide
MLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWFWVLVVVGGVLACYSLLVTVAFIIFWVKRGRKKLLYIFKQPFMRPVQTTQEEDGCSCRFPEEEEGGCELTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 39 - DNA, 2595 bp, Artificial Sequence
  Description of Artificial Sequence Synthetic polynucleotide
atgctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttggttttgggtgctggtggtggttggtggagtcctggcttgctatagcttgctagtaacagtggcctttattattttctgggtgctttgcgcacgcccacgccgcagccccgcccaagaagatggcaaagtctacatcaacatgccaggcaggggcacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 40 - protein, 864 aa, Artificial Sequence
  Description of Artificial Sequence Synthetic polypeptide
MLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWFWVLVVVGGVLACYSLLVTVAFIIFWVLCARPRRSPAQEDGKVYINMPGRGTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 41 - DNA, 2661 bp, Artificial Sequence
  Description of Artificial Sequence Synthetic polynucleotide
atgctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttggggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaaacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 42 - protein, 886 aa, Artificial Sequence
  Description of Artificial Sequence Synthetic polypeptide
MLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWGVLAGIVMGDLVLTVLIALAVYFLGRLVPRGRGAAEAATRKQRITETESPYQELQGQRSDVYSDLNTQRPYYKTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 43 - DNA, 2679 bp, Artificial Sequence
  Description of Artificial Sequence Synthetic polynucleotide
atgctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttggttttgggtgctggtggtggttggtggagtcctggcttgctatagcttgctagtaacagtggcctttattattttctgggtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaaacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 44 - protein, 892 aa, Artificial Sequence
  Description of Artificial Sequence Synthetic polypeptide
MLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWFWVLVVVGGVLACYSLLVTVAFIIFWVYFLGRLVPRGRGAAEAATRKQRITETESPYQELQGQRSDVYSDLNTQRPYYKTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 45 - DNA, 2658 bp, Artificial Sequence
  Description of Artificial Sequence Synthetic polynucleotide
atgctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggcggtgatgattatttttcgcattggcatggcggtggcgattttttgctgctttttttttccgagcgcgaacgaacgctgcaaaagcaaagtggtgccgtgccgccagaaacagtggcgcaccagctttgatagcaaaaaactggatctgaactataaccattttgaaagcatggaatggagccatcgcagccgccgcggccgcatttggggcatgacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 46 - protein, 885 aa, Artificial Sequence
  Description of Artificial Sequence Synthetic polypeptide
MLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWAVMIIFRIGMAVAIFCCFFFPSANERCKSKVVPCRQKQWRTSFDSKKLDLNYNHFESMEWSHRSRRGRIWGMTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 47 - DNA, 2676 bp, Artificial Sequence
  Description of Artificial Sequence Synthetic polynucleotide
atgctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttggttttgggtgctggtggtggttggtggagtcctggcttgctatagcttgctagtaacagtggcctttattattttctgggtgagcgcgaacgaacgctgcaaaagcaaagtggtgccgtgccgccagaaacagtggcgcaccagctttgatagcaaaaaactggatctgaactataaccattttgaaagcatggaatggagccatcgcagccgccgcggccgcatttggggcatgacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 48 - protein, 891 aa, Artificial Sequence
  Description of Artificial Sequence Synthetic polypeptide
MLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWFWVLVVVGGVLACYSLLVTVAFIIFWVSANERCKSKVVPCRQKQWRTSFDSKKLDLNYNHFESMEWSHRSRRGRIWGMTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 49 - protein, 18 aa, Thosea asigna virus
EGRGSLLTCGDVEENPGP

SEQ ID NO: 50 - protein, 19 aa, Teschovirus A
ATNFSLLKQAGDVEENPGP

SEQ ID NO: 51 - protein, 22 aa, Foot-and-mouth disease virus
VKQTLNFDLLKLAGDVESNPGP

SEQ ID NO: 52 - DNA, 3234 bp, Artificial Sequence
  Description of Artificial Sequence Synthetic polynucleotide
atgctcgagggagtgcaggtggaaaccatctccccaggcgacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagatcatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagcttcgcgagccgagggcaggggaagtcttctaacatgcggggacgtggaggaaaatcccgggcccctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacgattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgctttgcgcacgcccacgccgcagccccgcccaagaagatggcaaagtctacatcaacatgccaggcaggggcacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 53 - protein, 1077 aa, Artificial Sequence
  Description of Artificial Sequence Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAEGRGSLLTCGDVEENPGPLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTIEVMYPPPYLDNEKSNGTIIHVKGKHLCPSPLFPGPSKPFWVLVVVGGVLACYSLLVTVAFIIFWVLCARPRRSPAQEDGKVYINMPGRGTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 54 - DNA, 3285 bp, Artificial Sequence
  Description of Artificial Sequence Synthetic polynucleotide
atgctcgagggagtgcaggtggaaaccatctccccaggcgacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagatcatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagcttcgcgagccgagggcaggggaagtcttctaacatgcggggacgtggaggaaaatcccgggcccctcgagatggagttcggcttgagttggttgttccttgtggcgatactcaaaggcgttcaatgtagccgagacataaaaatgacccagtctccgtcatctatgtatgcaagcctcggcgagcgagtgaccatcacgtgcaaggcgagtcaagatataaacagctacttgtcatggttccaacaaaaaccagggaaatcacctaagaccctgatctatagagccaatcgcctggttgacggtgtcccctcccgctttagcggctccggaagcggtcaagattactctctcacaatttcttccttggattatgaagacatggggatctactattgtcaacagtatgacgaatccccgtggactttcggtggcggtaccaaattggaaataaagggctctacaagcggctcaggaaaacctggatcaggcgaagggtctacgaagggccagatacaactcgttcaaagtgggccagaactcaaaaaaccgggagaaacagtgaaaatttcttgtaaggcatcaggatacacattcacaaactacgggatgaattgggtcaaacaagcacccggaaaggggctgcgctggatggggtggatcaacacacatactggggaacctacttacgcagacgatttcaagggcagattcgccttttctttggagacctccgcctctactgcatacttgcagataaacaacctgaagaatgaagataccgccacctacttctgtacgcgcaggggctacgattggtattttgatgtatggggggcaggcaccactgttactgtgtcaagccgtacgattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcaacgcgtgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 55 - protein, 1094 aa, Artificial Sequence
  Description of Artificial Sequence Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAEGRGSLLTCGDVEENPGPLEMEFGLSWLFLVAILKGVQCSRDIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIKGSTSGSGKPGSGEGSTKGQIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSSRTIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS